(19) United States
(12) Patent Application Publication
(10) Pub. No.: US 2025/0297006 A1
(43) Pub. Date: Sep. 25, 2025

(54) Masked Multispecific Antigen-Binding Molecules with Cleavable Linkers

(71) Applicant: Regeneron Pharmaceuticals, Inc., Tarrytown, NY (US)

(72) Inventors: Lauric Haber, Rye Brook, NY (US); Jeff Glasgow, New York, NY (US); Eric Smith, New York, NY (US); Chia-Yang Lin, Scarsdale, NY (US); Tong Zhang, Fort Lee, NJ (US); Supriya Patel, Briarcliff Manor, NY (US); Emily Krueger, Elmsford, NY (US); Samuel Davis, New York, NY (US)

(21) Appl. No.: 19/085,162

(22) Filed: Mar. 20, 2025

Related U.S. Application Data
(60) Provisional application No. 63/567,636, filed on Mar. 20, 2024.
(60) Provisional application No. 63/642,152, filed on May 3, 2024.
(60) Provisional application No. 63/684,380, filed on Aug. 17, 2024.
(60) Provisional application No. 63/733,610, filed on Dec. 13, 2024.
(60) Provisional application No. 63/761,070, filed on Feb. 20, 2025.
(60) Provisional application No. 63/764,470, filed on Feb. 27, 2025.

(51) Int. Cl. C07K 16/28; A61P 35/00; C07K 16/30

(52) U.S. Cl. CPC C07K 16/2809; A61P 35/00; C07K 16/3076; C07K 2317/31; C07K 2317/522; C07K 2317/524; C07K 2317/526; C07K 2317/53; C07K 2317/55; C07K 2317/565

(57) ABSTRACT

The present disclosure provides multispecific antigen-binding molecules that bind both a T-cell antigen (e.g., CD3) and a target antigen (e.g., a tumor associated antigen), and which include tandem antigen-binding domains, and uses thereof.

### CROSS-REFERENCE TO RELATED APPLICATIONS

This application claims the benefit under 35 USC § 119(e) of U.S. Provisional Application No. 63/567,636, filed Mar. 20, 2024; 63/642,152, filed May 3, 2024; 63/684,380, filed Aug. 17, 2024; 63/733,610, filed Dec. 13, 2024; 63/761,070, filed Feb. 20, 2025; and 63/764,470, filed Feb. 27, 2025, each of which is incorporated herein by reference in its entirety for all purposes.

### REFERENCE TO A SEQUENCE LISTING

This application incorporates by reference a computer readable Sequence Listing in ST.26 XML format, titled 11325US01_Sequence, created on Mar. 20, 2025 and containing 496,633 bytes.

### FIELD OF THE DISCLOSURE

The present disclosure relates to alternative formats for multivalent antigen-binding proteins, and methods of use thereof. The multivalent antigen-binding proteins, including bispecific and multispecific molecules comprise stacked antigen-binding fragments (Fabs) that may be joined by a cleavable linker (e.g., a protease cleavable linker), in which one Fab specifically binds a target antigen (TA) and the second Fab specifically binds a T-cell antigen (TCA) (e.g., CD3).

### BACKGROUND

The selective destruction of tumor cells, while leaving healthy cells and tissues intact and undamaged, is a goal of cancer immunotherapy. Bispecific antibody therapeutics have been developed to achieve this goal by inducing an immune response against the tumor. In this regard, bispecific antibodies are designed to both a tumor-associated antigen (TAA) expressed preferentially on tumor cells and a component of the T cell receptor (TCR) complex. The simultaneous binding of such an antibody to both of its targets results in activation of cytotoxic T cells and subsequent lysis of the TAA-expressing cell. Hence, the immune response is re-directed to the TAA-expressing cells

Bispecific and multispecific antibodies and antigen-binding molecules are known in the art (see, e.g., Brinkmann and Kontermann, MABS, 9(2):182-212, 2017). Among such known formats is a traditional bispecific antibody with Fab antigen-binding domains on either arm of the antibody and an Fc region. This traditional bispecific antibody format has been used to make bispecific antibodies in which one arm of the antibody targets a tumor cell antigen and the second arm targets a T-cell antigen, such as CD3. Although Brinkmann et al. generically references the “building blocks” for the generation of any homodimeric or heterodimeric antigen-binding molecule (p. 183, FIG. 1), the possibilities are virtually infinite, and only those molecules shown in FIG. 2 (p. 184) had reportedly been prepared. Moreover, Brinkmann doesn't contemplate specific antigen-binding domains, particularly a molecule comprising stacked or tandem Fabs in which both Fabs comprise an identical light chain and are separated by a cleavable linker to mask the activity of the antigen-binding domain(s).

### BRIEF SUMMARY OF THE DISCLOSURE

In general, the present disclosure provides multispecific antigen-binding molecules that bind both a T-cell antigen (TCA) (e.g., CD3) and a target antigen (TA) (e.g., a tumor associated antigen), which include tandem Fabs that may be separated by a cleavable linker.

In one aspect, the present disclosure provides a multispecific antigen-binding molecule, comprising: (a) a first immunoglobulin antigen-binding fragment (Fab) comprising a first polypeptide comprising, from N-terminus to C-terminus, a light chain variable region (LCVR) and an immunoglobulin light chain constant region (CL), and a second polypeptide comprising, from N-terminus to C-terminus, a heavy chain variable region (HCVR) and an immunoglobulin CH1 heavy chain constant region (CH1), wherein the first polypeptide and the second polypeptide are connected by a disulfide bond; (b) a second immunoglobulin Fab comprising a first polypeptide comprising, from N-terminus to C-terminus, a LCVR and a CL, and a second polypeptide comprising, from N-terminus to C-terminus, a HCVR and a CH1; and (c) an immunoglobulin Fc region comprising a pair of polypeptides, each comprising a hinge region, an immunoglobulin CH2 heavy chain constant region (CH2) and an immunoglobulin CH3 heavy chain constant region (CH3), wherein the pair of polypeptides are connected by a disulfide bond or a non-cleavable linker, optionally wherein the pair of polypeptides is connected by a disulfide bond in the hinge region, wherein the first immunoglobulin Fab and the second immunoglobulin Fab are connected by a first linker between the C-terminus of the CL of the first immunoglobulin Fab and the N-terminus of the LCVR of the second immunoglobulin Fab, and by a second linker between the C-terminus of the CH1 of the first immunoglobulin Fab and the N-terminus of the HCVR of the second immunoglobulin Fab, and wherein the first linker or the second linker, but not both, is a protease cleavable linker, and wherein the first immunoglobulin Fab or the second immunoglobulin Fab, but not both, is connected to the immunoglobulin Fc region by a third linker that is a protease cleavable linker.

In some embodiments, the disulfide bond connecting the first polypeptide and the second polypeptide of the first immunoglobulin Fab is a disulfide bond between the CL to the CH1.

In some embodiments, the first polypeptide and the second polypeptide of the second immunoglobulin Fab are connected by a disulfide bond. In some cases, the disulfide bond connecting the first polypeptide and the second polypeptide of the second immunoglobulin Fab is a disulfide bond between the CL and the CH1. In some cases, the disulfide bond connecting the first polypeptide and the second polypeptide of the second immunoglobulin Fab is a disulfide bond between the LCVR and the HCVR. In some cases, the LCVR of the second immunoglobulin Fab comprises a cysteine mutation at residue 100 (Kabat numbering), and the HCVR of the second immunoglobulin Fab comprises a cysteine mutation at residue 44 (Kabat numbering).

In some embodiments, wherein the first polypeptide and the second polypeptide of the second immunoglobulin Fab are not connected by a disulfide bond.

In some embodiments, the first linker is a protease cleavable linker. In some embodiments, the second linker is a protease cleavable linker.

In some embodiments, the third linker connects the C-terminus of the CH1 of the second immunoglobulin Fab to the hinge region of the immunoglobulin Fc region. In some embodiments, the first immunoglobulin Fab or the second immunoglobulin Fab, but not both, is connected to the immunoglobulin Fc region by the third linker, and by a fourth linker that is a protease cleavable linker. In some cases, the third linker connects the C-terminus of the CL of the second immunoglobulin Fab to the hinge region of the immunoglobulin Fc region, and the fourth linker connects the C-terminus of the CH1 of the second immunoglobulin Fab to the hinge region of the immunoglobulin Fc region. In some cases, the third linker connects the N-terminus of the LCVR of the first immunoglobulin Fab to the CH3 of the immunoglobulin Fc region, and the fourth linker connects the N-terminus of the HCVR of the first immunoglobulin Fab to the CH3 of the immunoglobulin Fc region.

In some embodiments, the hinge region of each of the pair of polypeptides of the immunoglobulin Fc region is connected to an additional immunoglobulin Fab, wherein each of the additional immunoglobulin Fabs comprises a first polypeptide comprising, from N-terminus to C-terminus, a LCVR and a CL, and a second polypeptide comprising, from N-terminus to C-terminus, a HCVR and a CH1. In some embodiments, each of the additional immunoglobulin Fabs is connected, respectively, via the CH1 to the hinge region of one of the pair of polypeptides of the immunoglobulin Fc region. In some cases, each of the additional immunoglobulin Fabs binds an antigen distinct from that bound by the first immunoglobulin Fab and the second immunoglobulin Fab.

In some embodiments, the multispecific antigen-binding molecule further comprises a peptide mask connected to the N-terminus of the LCVR or the N-terminus of the HCVR of the first immunoglobulin Fab by a protease cleavable linker.

In some embodiments, the third linker connects the N-terminus of the HCVR of the first immunoglobulin Fab to the C-terminus of the CH3 of the immunoglobulin Fc region.

In some embodiments, the first immunoglobulin Fab specifically binds a target antigen. In some embodiments, the second immunoglobulin Fab specifically binds a T-cell antigen.

In some embodiments, the first immunoglobulin Fab specifically binds a T-cell antigen. In some embodiments, the second immunoglobulin Fab specifically binds a target antigen.

In some embodiments, the T-cell antigen is CD3.

In some embodiments, the target antigen is a tumor-associated antigen.

In some embodiments, the multispecific antigen-binding molecule further comprises: (c) a third immunoglobulin Fab comprising a first polypeptide comprising, from N-terminus to C-terminus, a LCVR and a CL, and a second polypeptide comprising, from N-terminus to C-terminus, a HCVR and a CH1; and (d) a fourth immunoglobulin Fab comprising a first polypeptide comprising, from N-terminus to C-terminus, a LCVR and a CL, and a second polypeptide comprising, from N-terminus to C-terminus, a HCVR and a CH1, wherein the third immunoglobulin Fab and the fourth immunoglobulin Fab are connected by a fifth linker between the C-terminus of the CL of the third immunoglobulin Fab and the N-terminus of the LCVR of the fourth immunoglobulin Fab, and by a sixth linker between the C-terminus of the CH1 of the third immunoglobulin Fab and the N-terminus of the HCVR of the fourth immunoglobulin Fab, and wherein the fifth linker or the sixth linker, but not both, is a protease cleavable linker, and wherein the third immunoglobulin Fab or the fourth immunoglobulin Fab, but not both, is connected to the immunoglobulin Fc region by a seventh linker that is a protease cleavable linker, and wherein (i) if the first immunoglobulin Fab is connected to the immunoglobulin Fc region, then the third immunoglobulin Fab is also connected to the immunoglobulin Fc region, or (ii) if the second immunoglobulin Fab is connected to the immunoglobulin Fc region, then the fourth immunoglobulin Fab is also connected to the immunoglobulin Fc region.

In some embodiments, the first linker and the fifth linker are protease cleavable linkers; the first linker and the sixth linker are protease cleavable linkers; the second linker and the fifth linker are protease cleavable linkers; or the second linker and the sixth linker are protease cleavable linkers.

In some embodiments, the third linker connects the C-terminus of the CH1 of the second immunoglobulin Fab to the hinge region of the immunoglobulin Fc region, and the seventh linker connects the C-terminus of the CH1 of the fourth immunoglobulin Fab to the hinge region of the immunoglobulin Fc region.

In some embodiments, the multispecific antigen-binding molecule further comprises: a first peptide mask connected to the N-terminus of the LCVR or the N-terminus of the HCVR of the first immunoglobulin Fab by a protease cleavable linker; and a second peptide mask connected to the N-terminus of the LCVR or the N-terminus of the HCVR of the third immunoglobulin Fab by a protease cleavable linker.

In some embodiments, the third linker connects the N-terminus of the HCVR of the first immunoglobulin Fab to the C-terminus of the CH3 of the immunoglobulin Fc region, and the seventh linker connects the N-terminus of the HCVR of the first immunoglobulin Fab to the C-terminus of the CH3 of the immunoglobulin Fc region.

In some embodiments, the first immunoglobulin Fab and the third immunoglobulin Fab specifically bind a target antigen. In some embodiments, the second immunoglobulin Fab and the fourth immunoglobulin Fab specifically bind a T-cell antigen.

In some embodiments, the first immunoglobulin Fab and the third immunoglobulin Fab specifically bind a T-cell antigen. In some embodiments, the second immunoglobulin Fab and the fourth immunoglobulin Fab specifically bind a target antigen.

In some embodiments, the T-cell antigen is CD3, and the target antigen is a tumor-associated antigen.

In one aspect, the present disclosure provides a multispecific antigen-binding molecule, comprising: (a) a first immunoglobulin antigen-binding fragment (Fab) comprising a first polypeptide comprising, from N-terminus to C-terminus, a light chain variable region (LCVR) and an immunoglobulin light chain constant region (CL), and a second polypeptide comprising, from N-terminus to C-terminus, a heavy chain variable region (HCVR) and an immunoglobulin CH1 heavy chain constant region (CH1), wherein the first polypeptide and the second polypeptide are connected by a disulfide bond; (b) a second immunoglobulin Fab comprising a first polypeptide comprising, from N-terminus to C-terminus, a LCVR and a CL, and a second polypeptide comprising, from N-terminus to C-terminus, a HCVR and a CH1; and (c) an immunoglobulin Fc region comprising a first polypeptide and a second polypeptide, each polypeptide comprising an immunoglobulin CH2 heavy chain constant region (CH2) and an immunoglobulin CH3 heavy chain constant region (CH3), wherein the first and second polypeptides are connected by a disulfide bond or a non-cleavable linker, wherein the first immunoglobulin Fab and the second immunoglobulin Fab are connected by a first linker between the C-terminus of the CL of the first immunoglobulin Fab and the N-terminus of the LCVR of the second immunoglobulin Fab, and by a second linker between the C-terminus of the CH1 of the first immunoglobulin Fab and the N-terminus of the HCVR of the second immunoglobulin Fab, and wherein the first linker is a non-cleavable linker and the second linker is a protease cleavable linker, and wherein the first immunoglobulin Fab is connected to the immunoglobulin Fc region by a third linker that is a protease cleavable linker between the N-terminus of the LCVR of the first immunoglobulin Fab and the C-terminus of the CH3 of the first polypeptide of the immunoglobulin Fc region, and by a fourth linker that is a protease cleavable linker between the N-terminus of the HCVR of the first immunoglobulin Fab and the C-terminus of the CH3 of the second polypeptide of the immunoglobulin Fc region.

In some cases, the first immunoglobulin Fab binds a T-cell antigen. In some cases, the second immunoglobulin Fab binds a target antigen. In some cases, the first immunoglobulin Fab binds a T-cell antigen, and the second immunoglobulin Fab binds a target antigen. In some cases, the first immunoglobulin Fab binds a target antigen. In some cases, the second immunoglobulin Fab binds a T-cell antigen. In some cases, the first immunoglobulin Fab binds a target antigen, and the second immunoglobulin Fab binds a T-cell antigen. In some embodiments, the T-cell antigen is CD3. In some embodiments, the target antigen is a tumor-associated antigen.

In some embodiments, the non-cleavable linker (a) consists of glycine and serine residues, or (b) is a linker selected from the linkers set forth in Table 9. In some cases, the non-cleavable linker is (G4S)₁. In some cases, the non-cleavable linker is (G4S)₂. In some cases, the non-cleavable linker is (G4S)₃.

In some embodiments, the protease cleavable linker is a linker comprising from 2 to 100 amino acids and containing a substrate for a protease. In some cases, the protease cleavable linker comprises from 2 to 50 amino acids, or from 5 to 50 amino acids, or from 2 to 25 amino acids, or from 5 to 25 amino acids, or from 2 to 20 amino acids, or from 5 to 20 amino acids, or from 2 to 15 amino acids, or from 5 to 15 amino acids, or from 2 to 10 amino acids, or from 5 to 10 amino acids.

In one aspect, the present disclosure provides a multispecific antigen-binding molecule, comprising: (a) a first immunoglobulin antigen-binding fragment (Fab) comprising: a first polypeptide comprising, from N-terminus to C-terminus, a peptide mask, a protease cleavable linker, a light chain variable region (LCVR) and an immunoglobulin light chain constant region (CL); and a second polypeptide comprising, from N-terminus to C-terminus, a peptide mask, a protease cleavable linker, a heavy chain variable region (HCVR) and an immunoglobulin CH1 heavy chain constant region (CH1), wherein the first polypeptide and the second polypeptide are connected by a disulfide bond; (b) a second immunoglobulin Fab comprising: a first polypeptide comprising, from N-terminus to C-terminus, a peptide mask, a protease cleavable linker, a LCVR and a CL; and a second polypeptide comprising, from N-terminus to C-terminus, a HCVR and a CH1, wherein the first polypeptide and the second polypeptide are connected by a disulfide bond; and (c) an immunoglobulin Fc region comprising a pair of polypeptides, each comprising a hinge region, an immunoglobulin CH2 heavy chain constant region (CH2) and an immunoglobulin CH3 heavy chain constant region (CH3), wherein the pair of polypeptides are connected by a disulfide bond or a non-cleavable linker, wherein the first immunoglobulin Fab and the second immunoglobulin Fab are connected by a non-cleavable linker between the C-terminus of the CH1 of the first immunoglobulin Fab and the N-terminus of the HCVR of the second immunoglobulin Fab, and wherein the first immunoglobulin Fab is connected to the immunoglobulin Fc region by a non-cleavable linker between the peptide mask of the second polypeptide and a CH3 of the immunoglobulin Fc region.

In one aspect, the present disclosure provides a multispecific antigen-binding molecule, comprising: (a) a first immunoglobulin antigen-binding fragment (Fab) comprising: a first polypeptide comprising, from N-terminus to C-terminus, a light chain variable region (LCVR) and an immunoglobulin light chain constant region (CL); and a second polypeptide comprising, from N-terminus to C-terminus, a peptide mask, a protease cleavable linker, a heavy chain variable region (HCVR) and an immunoglobulin CH1 heavy chain constant region (CH1), wherein the first polypeptide and the second polypeptide are connected by a disulfide bond; (b) a second immunoglobulin Fab comprising: a first polypeptide comprising, from N-terminus to C-terminus, a LCVR and a CL; and a second polypeptide comprising, from N-terminus to C-terminus, a HCVR and a CH1, wherein the first polypeptide and the second polypeptide are connected by a disulfide bond; and (c) an immunoglobulin Fc region comprising a pair of polypeptides, each comprising a hinge region, an immunoglobulin CH2 heavy chain constant region (CH2) and an immunoglobulin CH3 heavy chain constant region (CH3), wherein the pair of polypeptides are connected by a disulfide bond or a non-cleavable linker, wherein the first immunoglobulin Fab and the second immunoglobulin Fab are connected by a non-cleavable linker between the C-terminus of the CH1 of the first immunoglobulin Fab and the N-terminus of the HCVR of the second immunoglobulin Fab, and wherein the first immunoglobulin Fab is connected to the immunoglobulin Fc region by a non-cleavable linker between the peptide mask of the second polypeptide and a CH3 of the immunoglobulin Fc region.

In one aspect, the present disclosure provides a multispecific antigen-binding molecule, comprising: (a) a first immunoglobulin antigen-binding fragment (Fab) comprising: a first polypeptide comprising, from N-terminus to C-terminus, a peptide mask, a protease cleavable linker, a light chain variable region (LCVR) and an immunoglobulin light chain constant region (CL); and a second polypeptide comprising, from N-terminus to C-terminus, a heavy chain variable region (HCVR) and an immunoglobulin CH1 heavy chain constant region (CH1), wherein the first polypeptide and the second polypeptide are connected by a disulfide bond; (b) a second immunoglobulin Fab comprising: a first polypeptide comprising, from N-terminus to C-terminus, a peptide mask, a protease cleavable linker, a LCVR and a CL; and a second polypeptide comprising, from N-terminus to C-terminus, a HCVR and a CH1, wherein the first polypeptide and the second polypeptide are connected by a disulfide bond; and (c) an immunoglobulin Fc region comprising a pair of polypeptides, each comprising a hinge region, an immunoglobulin CH2 heavy chain constant region (CH2) and an immunoglobulin CH3 heavy chain constant region (CH3), wherein the pair of polypeptides are connected by a disulfide bond or a non-cleavable linker, wherein the first immunoglobulin Fab and the second immunoglobulin Fab are connected by a non-cleavable linker between the C-terminus of the CH1 of the first immunoglobulin Fab and the N-terminus of the HCVR of the second immunoglobulin Fab, and

wherein the first immunoglobulin Fab is connected to the immunoglobulin Fc region by a protease cleavable linker between the HCVR of the second polypeptide and a CH3 of the immunoglobulin Fc region.

In some embodiments, the immunoglobulin Fc region is an immunoglobulin Fc region of an antibody, wherein the antibody comprises two Fabs, each comprising a first polypeptide comprising, from N-terminus to C-terminus, a peptide mask, a protease cleavable linker, a LCVR and a CL, and a second polypeptide comprising a HCVR and a CH1, wherein the first and second polypeptide are connected by a disulfide bond, and the wherein the CH1 of each of the two Fabs is connected to the hinge region of the immunoglobulin Fc region.

In some embodiments, the first immunoglobulin Fab binds a T-cell antigen, and the second immunoglobulin Fab binds a target antigen. In some cases, the first immunoglobulin Fab binds a target antigen, and the second immunoglobulin Fab binds a T-cell antigen. In some embodiments, the target antigen is a tumor-associated antigen. In some embodiments, the T-cell antigen is CD3.

In one aspect, the present disclosure provides a multispecific antigen-binding molecule, comprising: (a) a first immunoglobulin antigen-binding fragment (Fab) comprising: a first polypeptide comprising, from N-terminus to C-terminus, a peptide mask, a protease cleavable linker, a light chain variable region (LCVR) and an immunoglobulin light chain constant region (CL); and a second polypeptide comprising, from N-terminus to C-terminus, a peptide mask, a protease cleavable linker, a heavy chain variable region (HCVR) and an immunoglobulin CH1 heavy chain constant region (CH1), wherein the first polypeptide and the second polypeptide are connected by a disulfide bond; (b) a second immunoglobulin Fab comprising: a first polypeptide comprising, from N-terminus to C-terminus, a peptide mask, a protease cleavable linker, a LCVR and a CL; and a second polypeptide comprising, from N-terminus to C-terminus, a HCVR and a CH1, wherein the first polypeptide and the second polypeptide are connected by a disulfide bond; (c) a third immunoglobulin Fab comprising: a first polypeptide comprising, from N-terminus to C-terminus, a peptide mask, a protease cleavable linker, a LCVR and a CL; and a second polypeptide comprising, from N-terminus to C-terminus, a peptide mask, a protease cleavable linker, a HCVR and a CH1, wherein the first polypeptide and the second polypeptide are connected by a disulfide bond; (d) a fourth immunoglobulin Fab comprising: a first polypeptide comprising, from N-terminus to C-terminus, a peptide mask, a protease cleavable linker, a LCVR and a CL; and a second polypeptide comprising, from N-terminus to C-terminus, a HCVR and a CH1, wherein the first polypeptide and the second polypeptide are connected by a disulfide bond; and (e) an immunoglobulin Fc region comprising a pair of polypeptides, each comprising a hinge region, an immunoglobulin CH2 heavy chain constant region (CH2) and an immunoglobulin CH3 heavy chain constant region (CH3), wherein the pair of polypeptides are connected by a disulfide bond or a non-cleavable linker, wherein the first immunoglobulin Fab and the second immunoglobulin Fab are connected by a non-cleavable linker between the C-terminus of the CH1 of the first immunoglobulin Fab and the N-terminus of the HCVR of the second immunoglobulin Fab, wherein the third immunoglobulin Fab and the fourth immunoglobulin Fab are connected by a non-cleavable linker between the C-terminus of the CH1 of the third immunoglobulin Fab and the N-terminus of the HCVR of the fourth immunoglobulin Fab, wherein the first immunoglobulin Fab is connected to the immunoglobulin Fc region by a non-cleavable linker between the peptide mask of the second polypeptide and a CH3 of the immunoglobulin Fc region, and wherein the third immunoglobulin Fab is connected to the immunoglobulin Fc region by a non-cleavable linker between the peptide mask of the second polypeptide and a CH3 of the immunoglobulin Fc region.

In one aspect, the present disclosure provides a multispecific antigen-binding molecule, comprising: (a) a first immunoglobulin antigen-binding fragment (Fab) comprising: a first polypeptide comprising, from N-terminus to C-terminus, a light chain variable region (LCVR) and an immunoglobulin light chain constant region (CL); and a second polypeptide comprising, from N-terminus to C-terminus, a peptide mask, a protease cleavable linker, a heavy chain variable region (HCVR) and an immunoglobulin CH1 heavy chain constant region (CH1), wherein the first polypeptide and the second polypeptide are connected by a disulfide bond; (b) a second immunoglobulin Fab comprising: a first polypeptide comprising, from N-terminus to C-terminus, a LCVR and a CL; and a second polypeptide comprising, from N-terminus to C-terminus, a HCVR and a CH1, wherein the first polypeptide and the second polypeptide are connected by a disulfide bond; (c) a third immunoglobulin Fab comprising: a first polypeptide comprising, from N-terminus to C-terminus, a LCVR and a CL; and a second polypeptide comprising, from N-terminus to C-terminus, a peptide mask, a protease cleavable linker, a HCVR and a CH1, wherein the first polypeptide and the second polypeptide are connected by a disulfide bond; (d) a fourth immunoglobulin Fab comprising: a first polypeptide comprising, from N-terminus to C-terminus, a LCVR and a CL; and a second polypeptide comprising, from N-terminus to C-terminus, a HCVR and a CH1, wherein the first polypeptide and the second polypeptide are connected by a disulfide bond; and (e) an immunoglobulin Fc region comprising a pair of polypeptides, each comprising a hinge region, an immunoglobulin CH2 heavy chain constant region (CH2) and an immunoglobulin CH3 heavy chain constant region (CH3), wherein the pair of polypeptides are connected by a disulfide bond or a non-cleavable linker, wherein the first immunoglobulin Fab and the second immunoglobulin Fab are connected by a non-cleavable linker between the C-terminus of the CH1 of the first immunoglobulin Fab and the N-terminus of the HCVR of the second immunoglobulin Fab, wherein the third immunoglobulin Fab and the fourth immunoglobulin Fab are connected by a non-cleavable linker between the C-terminus of the CH1 of the third immunoglobulin Fab and the N-terminus of the HCVR of the fourth immunoglobulin Fab, wherein the first immunoglobulin Fab is connected to the immunoglobulin Fc region by a non-cleavable linker between the peptide mask of the second polypeptide and a CH3 of the immunoglobulin Fc region, and wherein the third immunoglobulin Fab is connected to the immunoglobulin Fc region by a non-cleavable linker between the peptide mask of the second polypeptide and a CH3 of the immunoglobulin Fc region.

In one aspect, the present disclosure provides a multispecific antigen-binding molecule, comprising: (a) a first immunoglobulin antigen-binding fragment (Fab) comprising: a first polypeptide comprising, from N-terminus to C-terminus, a peptide mask, a protease cleavable linker, a light chain variable region (LCVR) and an immunoglobulin light chain constant region (CL); and a second polypeptide comprising, from N-terminus to C-terminus, a heavy chain variable region (HCVR) and an immunoglobulin CH1 heavy chain constant region (CH1), wherein the first polypeptide and the second polypeptide are connected by a disulfide bond; (b) a second immunoglobulin Fab comprising: a first polypeptide comprising, from N-terminus to C-terminus, a peptide mask, a protease cleavable linker, a LCVR and a CL; and a second polypeptide comprising, from N-terminus to C-terminus, a HCVR and a CH1, wherein the first polypeptide and the second polypeptide are connected by a disulfide bond; (c) a third immunoglobulin Fab comprising: a first polypeptide comprising, from N-terminus to C-terminus, a peptide mask, a protease cleavable linker, a LCVR and a CL; and a second polypeptide comprising, from N-terminus to C-terminus, a HCVR and a CH1, wherein the first polypeptide and the second polypeptide are connected by a disulfide bond; (d) a fourth immunoglobulin Fab comprising: a first polypeptide comprising, from N-terminus to C-terminus, a peptide mask, a protease cleavable linker, a LCVR and a CL; and a second polypeptide comprising, from N-terminus to C-terminus, a HCVR and a CH1, wherein the first polypeptide and the second polypeptide are connected by a disulfide bond; and (e) an immunoglobulin Fc region comprising a pair of polypeptides, each comprising a hinge region, an immunoglobulin CH2 heavy chain constant region (CH2) and an immunoglobulin CH3 heavy chain constant region (CH3), wherein the pair of polypeptides are connected by a disulfide bond or a non-cleavable linker, wherein the first immunoglobulin Fab and the second immunoglobulin Fab are connected by a non-cleavable linker between the C-terminus of the CH1 of the first immunoglobulin Fab and the N-terminus of the HCVR of the second immunoglobulin Fab, wherein the third immunoglobulin Fab and the fourth immunoglobulin Fab are connected by a non-cleavable linker between the C-terminus of the CH1 of the third immunoglobulin Fab and the N-terminus of the HCVR of the fourth immunoglobulin Fab, wherein the first immunoglobulin Fab is connected to the immunoglobulin Fc region by a protease cleavable linker between the HCVR of the second polypeptide and a CH3 of the immunoglobulin Fc region, and wherein the third immunoglobulin Fab is connected to the immunoglobulin Fc region by a protease cleavable linker between the HCVR of the second polypeptide and a CH3 of the immunoglobulin Fc region.

In some embodiments, the immunoglobulin Fc region is an immunoglobulin Fc region of an antibody, wherein the antibody comprises two Fabs, each comprising a first polypeptide comprising, from N-terminus to C-terminus, a peptide mask, a protease cleavable linker, a LCVR and a CL, and a second polypeptide comprising a HCVR and a CH1, wherein the first and second polypeptide are connected by a disulfide bond, and the wherein the CH1 of each of the two Fabs is connected to the hinge region of the immunoglobulin Fc region.

In some embodiments, the first immunoglobulin Fab and the third immunoglobulin Fab bind a T-cell antigen, and the second immunoglobulin Fab and the fourth immunoglobulin Fab bind a target antigen. In some embodiments, the first immunoglobulin Fab and the third immunoglobulin Fab bind a target antigen, and the second immunoglobulin Fab and the fourth immunoglobulin Fab bind a T-cell antigen. In some embodiments, the target antigen is a tumor-associated antigen. In some embodiments, the T-cell antigen is a CD3.

In the embodiments discussed above or herein, each polypeptide comprising (i) a peptide mask, a protease cleavable linker, a LCVR and a CL, or (ii) a LCVR and a CL, is identical.

In the embodiments discussed above or herein, each peptide mask is an anti-idiotype antigen-binding molecule that binds either a target antigen binding domain of a Fab or a T-cell antigen binding domain of a Fab, but not both.

In the embodiments discussed above or herein, which include an immunoglobulin Fc region that is part of an antibody, the antibody may bind to a tumor-associated antigen.

In the embodiments discussed above or herein, the non-cleavable linker (a) consists of glycine and serine residues, or (b) is a linker selected from the linkers set forth in Table 9.

In the embodiments discussed above or herein, the protease cleavable linker is a linker comprising from 2 to 100 amino acids and containing a substrate for a protease. In some cases, the protease cleavable linker comprises from 2 to 50 amino acids, or from 5 to 50 amino acids, or from 2 to 25 amino acids, or from 5 to 25 amino acids, or from 2 to 20 amino acids, or from 5 to 20 amino acids, or from 2 to 15 amino acids, or from 5 to 15 amino acids, or from 2 to 10 amino acids, or from 5 to 10 amino acids.

In one aspect, the present disclosure provides a multispecific antigen-binding molecule comprising the structure of any one of FIGS. 1A, 1B, 1C, 1D, 1E, 1F, 1G, 2A, 2B, 3A, 3B, 4A, 4B, 4C, 4D, 4E, 4F, 4G, 4H, 4I, 5A, 5B, 5C, 5D, 6A, 6B, 7A, 7B, 8A, 8B, 8C, 8D, 8E, 8F, 8G, 8H, 8I, 8J, 8K, 8L, 9A, 9B, 9C, 10A, 10B, 10C, 10D, 11A, 11B, 11C and 11D.

In any of the various embodiments discussed above or herein, the multispecific antigen-binding molecule may be a bispecific antigen-binding molecule.

In one aspect, the present disclosure provides a nucleic acid or plurality of nucleic acids encoding the multispecific antigen-binding molecule discussed above or herein.

In one aspect, the present disclosure provides an isolated cell transfected with one or more expression vectors comprising one or more nucleic acid sequences encoding the multispecific antigen-binding molecule discussed above or herein.

In one aspect, the present disclosure provides a method of producing the multispecific antigen-binding molecule discussed above or herein, comprising: (a) culturing a host cell as discussed above or herein under conditions in which the multispecific antigen-binding molecule is expressed; and (b) recovering the multispecific antigen-binding molecule from the cell culture. In some cases, the method further comprises formulating the multispecific antigen-binding molecule with a pharmaceutically acceptable excipient or diluent. In some cases, the method further comprises purifying the multispecific antigen-binding molecule prior to formulating the multispecific antigen-binding molecule with a pharmaceutically acceptable excipient or diluent.

In one aspect, the present disclosure provides a pharmaceutical composition comprising the multispecific antigen-binding molecule as discussed above or herein, and a pharmaceutically acceptable carrier or diluent.

In one aspect, the present disclosure provides a method of treating cancer, comprising administering the multispecific antigen-binding molecule or the pharmaceutical composition discussed above or herein to a subject in need thereof.

In any of the above methods, the multispecific antigen-binding molecule may be administered in combination with a second therapeutic agent.

In one aspect, the present disclosure a polypeptide comprising, from N-terminus to C-terminus: (a) a peptide mask; (b) a first protease cleavable linker; (c) a first immunoglobulin light chain comprising a light chain variable region (LCVR) and a light chain constant region (CL); (d) a second protease cleavable linker; and (e) a second immunoglobulin light chain comprising a LCVR and a CL.

In some embodiments of the polypeptide discussed above or herein, the first immunoglobulin light chain is identical to the second immunoglobulin light chain. In some embodiments, the first immunoglobulin light chain is paired with a first heavy chain variable region (HCVR) and a first immunoglobulin CH1 heavy chain constant region (CH1), and the second immunoglobulin light chain is paired with a second HCVR and CH1, wherein at least one of the first immunoglobulin light chain or the second immunoglobulin light chain is connected to the paired HCVR and CH1 by a disulfide bond. In some cases, the first HCVR is different from the second HCVR. In some embodiments, the first immunoglobulin light chain and/or the second immunoglobulin light chain comprises a LCVR comprising the amino acid sequence of SEQ ID NO: 162.

In some embodiments of the polypeptide discussed above or herein, the protease cleavable linker is a linker comprising from 2 to 100 amino acids and containing a substrate for a protease. In some cases, the protease cleavable linker comprises from 2 to 50 amino acids, or from 5 to 50 amino acids, or from 2 to 25 amino acids, or from 5 to 25 amino acids, or from 2 to 20 amino acids, or from 5 to 20 amino acids, or from 2 to 15 amino acids, or from 5 to 15 amino acids, or from 2 to 10 amino acids, or from 5 to 10 amino acids.

In one aspect, the present disclosure provides a nucleic acid or plurality of nucleic acids encoding the polypeptide discussed above or herein.

In one aspect, the present disclosure provides an isolated cell transfected with one or more expression vectors comprising one or more nucleic acid sequences encoding the polypeptide as discussed above or herein.

In one aspect, the present disclosure provides a pharmaceutical composition comprising the polypeptide as discussed above or herein, and a pharmaceutically acceptable carrier or diluent.

In one aspect, the present disclosure provides a method of treating cancer, comprising administering the polypeptide or the pharmaceutical composition as discussed above or herein to a subject in need thereof.

In any of the above methods, the polypeptide may be administered in combination with a second therapeutic agent.

In various embodiments, the target antigen is present at a density of from 10 to 10,000,000 copies per target cell. In various embodiments, the target antigen is present at a density of from 50 to 10,000,000 copies per target cell. In various embodiments, the target antigen is present at a density of from 100 to 10,000,000 copies per target cell. In various embodiments, the target antigen is present at a density of from 10 to 1,000,000, of from 50 to 1,000,000, or from 100 to 1,000,000 copies per target cell. In some embodiments, the target antigen is present at a density of from 10 to 10,000, or from 50 to 10,000, or from 100 to 10.000. In some embodiments, the target antigen is present at a density of from 10 to 5000, or from 50 to 5000, or from 100 to 5000. In some embodiments, the target antigen is present at a density of from 50 to 20,000, or from 100 to 20,000. In some embodiments, the target antigen is present at a density of from 500 to 1,000,000 copies per target cell. In some embodiments, the target antigen is present at a density of from 1000 to 20,000 copies per target cell. In some embodiments, the target antigen is present at a density of greater than 20,000 copies per target cell. In various embodiments, the target antigen is present at a density of about 10, about 50, about 100, about 200, about 300, about 400, about 500, about 1000, about 2000, about 3000, about 4000, about 5000, about 6000, about 7000, about 8000, about 9000, about 10,000, about 15,000, about 20,000, about 25,000, about 50,000, about 75,000, about 100,000, about 200,000, about 300,000, about 400,000, about 500,000, about 600,000, about 700,000, about 800,000, about 900,000, about 1,000,000, about 2,000,000, about 3,000,000, about 4,000,000 or about 5,000,000 copies per target cell. As used herein, a “low density antigen” is an antigen where no more than 5000 copies of the antigen are found on a target cell. References to a low density antigen include cases in which a cell has no more than 4000, no more than 3000, no more than 2000, no more than 1000, no more than 900, no more than 800, no more than 700, no more than 600, no more than 500, no more than 400, no more than 300, no more than 200, no more than 100, or no more than 50 copies of the target antigen.

Any of the embodiments of the treatment methods discussed above or herein (e.g., for treating a cancer) are intended to, and do, encompass use of the molecule(s) in the manufacture of a medicament for treatment, as well as the molecule(s) (or pharmaceutical composition comprising the molecule(s)) for use in treatment.

In various embodiments, any of the features or components of embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges are encompassed within the scope of the present disclosure.

Other embodiments will become apparent from a review of the ensuing detailed description.

### BRIEF DESCRIPTION OF THE DRAWINGS

FIGS. 1A and 1B illustrate an embodiment of the multispecific antigen-binding molecules of the present disclosure in which tandem Fabs are separated by a protease cleavable linker (CLV) between the tandem light chains and in which the tandem Fabs are linked to an immunoglobulin hinge and Fc region by a protease cleavable linker (FIG. 1A), and whereupon exposure to a protease as discussed in Example 1, the linkers are cleaved to generate an activated tandem Fab in which the binding domains of both Fabs are able to bind to their respective antigens (e.g., a target antigen, such as a tumor-associated antigen, and a T-cell antigen such as CD3).

FIGS. 1C, 1D and 1E illustrate alternative embodiments of the multispecific antigen-binding molecules of the present disclosure, in which a pair of tandem Fabs are linked to an immunoglobulin hinge and Fc region by protease cleavable linkers, respectively (FIG. 1C), or in which tandem Fabs are linked to an immunoglobulin hinge and Fc region by two protease cleavable linkers (FIG. 1D and FIG. 1E). FIGS. 1D and 1E illustrate the presence of a cleavable linker between the light chain portions of the two Fabs (FIG. 1D) or the heavy chain portions of the two Fabs (FIG. 1E).

FIGS. 1F and 1G illustrate alternative embodiments of the multispecific antigen-binding molecules of the present disclosure, in which tandem Fabs are linked to an immunoglobulin hinge and Fc region by two protease cleavable linkers, and in which the Fab not linked to the hinge and Fc region is linked to a peptide mask by a protease cleavable linker, which may be connected to either the light chain portion of the molecule (FIG. 1F) or the heavy chain portion of the molecule (FIG. 1G). In these embodiments, the antigen-binding domains of both Fabs are masked until cleavage of the protease cleavable linkers.

FIGS. 2A and 2B illustrate an embodiment of the multispecific antigen-binding molecules of the present disclosure (like those of FIGS. 1A and 1B) in which the protease cleavable linker between the tandem Fabs is located between the heavy chain portions (HCVR and CH1 domains) of the tandem Fabs.

FIGS. 3A and 3B illustrate an embodiment of the multispecific antigen-binding molecules of the present disclosure (like those of FIGS. 1A and 1B) in which the Fab not linked to the hinge and Fc region is linked to a peptide mask by a protease cleavable linker. In this embodiment, the antigen-binding domains of both Fabs are masked until cleavage of the protease cleavable linkers.

FIGS. 4A and 4B illustrate embodiments of the multispecific antigen-binding molecules of the present disclosure like that of FIG. 1A, but in which the immunoglobulin hinge and Fc region is connected to the N-terminus of one of the tandem Fabs rather than the C-terminus (as in FIG. 1A). FIGS. 4A and 4B illustrate the presence of a cleavable linker between the light chain portions of the two Fabs (FIG. 4A) or the heavy chain portions of the two Fabs (FIG. 4B).

FIG. 4C illustrates an embodiment of the multispecific antigen-binding molecules of the present disclosure like that of FIG. 1C, but in which the immunoglobulin hinge and Fc region is connected to the N-terminus of each of the tandem Fabs rather than the C-terminus (as in FIG. 1C).

FIGS. 4D and 4E illustrate embodiments of the multispecific antigen-binding molecules of the present disclosure like those of FIGS. 1D and 1E, but in which the immunoglobulin hinge and Fc region is connected to the N-terminus of one of the tandem Fabs rather than the C-terminus (as in FIGS. 1D and 1E). FIGS. 4D and 4E illustrate the presence of a cleavable linker between the light chain portions of the two Fabs (FIG. 4D) or the heavy chain portions of the two Fabs (FIG. 4E).

FIGS. 4F and 4G illustrate embodiments of the multispecific antigen-binding molecules of the present disclosure (like those of FIGS. 4D and 4E) in which the immunoglobulin hinge and Fc region is part of an antibody (anti-TA* antibody). FIGS. 4F and 4G illustrate the presence of a cleavable linker between the light chain portions of the two Fabs (FIG. 4F) or the heavy chain portions of the two Fabs (FIG. 4G).

FIGS. 4H and 4I illustrate embodiments of the multispecific antigen-binding molecules of the present disclosure that result from exposure of the molecules of FIGS. 4D, 4E, 4F or 4G to a protease as discussed in Example 1. The linkers are cleaved to generate an activated tandem Fab in which the binding domains of both Fabs are able to bind to their respective antigens (e.g., a target antigen, such as a tumor-associated antigen, and a T-cell antigen such as CD3). The molecule of FIG. 4H corresponds to the molecules of FIGS. 4D and 4F, while the molecule of FIG. 4I corresponds to the molecules of FIGS. 4E and 4G.

FIGS. 5A, 5B, 5C and 5D illustrate embodiments of the multispecific antigen-binding molecules of the present disclosure like those of FIGS. 1A, 1B, 2A and 2B, but in which a disulfide bond connects the variable region portions of the internal Fab. In some embodiments, this disulfide bond may connect a LCVR comprising a cysteine mutation at residue 100 (Kabat numbering) with a HCVR comprising a cysteine mutation at residue 44 (Kabat numbering).

FIGS. 6A, 6B, 7A and 7B illustrate embodiments of the multispecific antigen-binding molecules of the present disclosure (like FIGS. 1A and 2A), in which there is no disulfide bond connecting the light chain and heavy chain portions of the internal Fab. While the light chain and heavy chain portions of the internal Fab may be associated with one another via non-covalent molecular interactions, this association may be temporally limited such that following cleavage of the protease cleavable linker connecting the tandem Fabs, the (formerly) internal Fab may disassociate based on a half-life resulting in loss of function for the antigen-binding domain of the (formerly) internal Fab.

FIGS. 8A, 8B and 8C illustrate embodiments of the multispecific antigen-binding molecules of the present disclosure, in which tandem Fabs are linked to an immunoglobulin hinge and Fc region by a protease cleavable linker and a peptide mask, and in which the two tandem Fabs are connected by a non-cleavable linker. In the embodiments illustrated in FIGS. 8A and 8B, a common light chain linked to a peptide mask via a protease cleavable linker forms the light chain portion of each of the two Fabs. The peptide mask linked to the light chain (at the LCVR) may be an anti-idiotype binding domain such that the peptide mask inhibits the binding ability of only the target antigen binding domain (e.g., a tumor-associated antigen) as shown in FIG. 8A or the T-cell antigen (e.g., CD3) binding domain, as shown in FIG. 8B, while the peptide mask forming the linkage between the tandem Fabs and the hinge and Fc region may have the opposite anti-idiotype binding affinity (i.e., binding to the anti-CD3 binding domain in the molecule of FIG. 8A, and binding to the anti-TA binding domain in the molecule of FIG. 8B). Protease cleavage of the various cleavable linkers in the molecule of FIG. 8A or 8B yields the molecule shown in FIG. 8C.

FIGS. 8D, 8E and 8F illustrate embodiments of the multispecific antigen-binding molecules of the present disclosure, in which tandem Fabs are linked to an immunoglobulin hinge and Fc region by a protease cleavable linker and a peptide mask, and in which the two tandem Fabs are connected by a non-cleavable linker. In contrast to the molecules illustrated in FIGS. 8A and 8B, the molecules illustrated in FIGS. 8D and 8E do not include peptide masks linked to the light chains. In the embodiments illustrated in FIGS. 8D and 8E, a common light chain forms the light chain portion of each of the two Fabs. The peptide mask linked to the heavy chain (at the HCVR) may be an anti-idiotype binding domain such that the peptide mask inhibits the binding ability of the target antigen binding domain (e.g., a tumor-associated antigen) as shown in FIG. 8E or the T-cell antigen (e.g., CD3) binding domain, as shown in FIG. 8D. Protease cleavage of the cleavable linker in the molecule of FIG. 8D or 8E yields the molecule shown in FIG. 8F.

FIGS. 8G, 8H, 8I, 8J, 8K and 8L illustrate embodiments of the multispecific antigen-binding molecules of the present disclosure, in which tandem Fabs are linked to an immunoglobulin hinge and Fc region by a protease cleavable linker, and in which the two tandem Fabs are connected by a non-cleavable linker. In contrast to the molecules illustrated in FIGS. 8A and 8B, the molecules illustrated in FIGS. 8G and 8H do not include peptide masks linked to the heavy chains. In the embodiments illustrated in FIGS. 8G, 8H, 8J and 8K, a common light chain linked to a peptide mask via a protease cleavable linker forms the light chain portion of each of the two Fabs. The peptide mask linked to the light chain (at the LCVR) may be an anti-idiotype binding domain such that the peptide mask inhibits the binding ability of only the target antigen binding domain (e.g., a tumor-associated antigen) as shown in FIGS. 8G and 8K or the T-cell antigen (e.g., CD3) binding domain, as shown in FIGS. 8H and 8J. Protease cleavage of the various cleavable linkers in the molecule of FIG. 8G or 8H yields the molecule shown in FIG. 8I, while protease cleavage of the various cleavable linkers in the molecules of FIG. 8J or 8K yields to the molecule shown in FIG. 8L.

FIGS. 9A, 9B and 9C illustrate embodiments of the multispecific antigen-binding molecules of the present disclosure (like those of FIGS. 8A, 8B and 8C), in which the immunoglobulin hinge and Fc region in the molecules shown in FIGS. 9A and 9B is part of an antibody (anti-TA* antibody) that may be used for targeting the molecule to the tumor environment where protease cleavage of the cleavable linkers may occur. Protease cleavage of the various cleavable linkers in the molecule of FIG. 9A or 9B yields the molecule shown in FIG. 9C. Molecules like those illustrated in FIGS. 9A and 9B, but without the light chain linked peptide masks in the tandem Fabs (as shown in FIGS. 8D and 8E) are also encompassed within the present disclosure. Molecules like those illustrated in FIGS. 9A and 9B, but without the heavy chain linked peptide masks in the tandem Fabs (as shown in FIGS. 8G, 8H, 8J and 8K) are also encompassed within the present disclosure.

FIGS. 10A, 10B, 10C and 10D illustrate embodiments of the multispecific antigen-binding molecules of the present disclosure, in which a pair of tandem Fabs are linked to an immunoglobulin hinge and Fc region by a pair of protease cleavable linkers and peptide masks, respectively, and in which the tandem Fabs in each pair are connected by a non-cleavable linker. In the embodiments illustrated in FIGS. 10A and 10C, a common light chain linked to a peptide mask via a protease cleavable linker forms the light chain portion of each of the Fabs. The peptide mask linked to the light chain (at the LCVR) may be an anti-idiotype binding domain such that the peptide mask inhibits the binding ability of only the target antigen binding domain (e.g., a tumor-associated antigen) as shown in FIG. 10A or the T-cell antigen (e.g., CD3) binding domain, as shown in FIG. 10C, while the peptide mask forming the linkage between each of the tandem Fabs and the hinge and Fc region may have the opposite anti-idiotype binding affinity (i.e., binding to the anti-CD3 binding domain in the molecule of FIG. 10A, and binding to the anti-TA binding domain in the molecule of FIG. 10C). Protease cleavage of the various cleavable linkers in the molecule of FIG. 10A yields the molecule shown in FIG. 10B, while protease cleavage of the various cleavable linkers in the molecule of FIG. 10C yields the molecule shown in FIG. 10D. Where the tandem Fabs of the molecules shown in FIGS. 10A and 10C are homodimeric (as shown), protease cleavage yields a pair of the molecules shown in FIGS. 10B and 10D (represented by the “(x2)”) in the figures. Molecules like those illustrated in FIGS. 10A and 10C, but without the light chain linked peptide masks in the pair of tandem Fabs (as shown in FIGS. 8D and 8E) are also encompassed within the present disclosure. Molecules like those illustrated in FIGS. 10A and 10C, but without the heavy chain linked peptide masks in the tandem Fabs (as shown in FIGS. 8G, 8H, 8J and 8K) are also encompassed within the present disclosure.

FIGS. 11A, 11B, 11C and 11D illustrate embodiments of the multispecific antigen-binding molecules of the present disclosure (like those of FIGS. 10A, 10B, 10C and 10D), in which the immunoglobulin hinge and Fc region in the molecules shown in FIGS. 11A and 11C is part of an antibody (anti-TA* antibody) that may be used for targeting the molecule to the tumor environment where protease cleavage of the cleavable linkers may occur. Protease cleavage of the various cleavable linkers in the molecule of FIG. 11A yields the molecule shown in FIG. 11B, while protease cleavage of the various cleavable linkers in the molecule of FIG. 11C yields the molecule shown in FIG. 11D. Where the tandem Fabs of the molecules shown in FIGS. 11A and 11C are homodimeric (as shown), protease cleavage yields a pair of the molecules shown in FIGS. 11B and 11D (represented by the “(x2)”) in the figures. Molecules like those illustrated in FIGS. 11A and 11C, but without the light chain linked peptide masks in the tandem Fabs (as shown in FIGS. 8D and 8E) are also encompassed within the present disclosure. Molecules like those illustrated in FIGS. 11A and 11C, but without the heavy chain linked peptide masks in the tandem Fabs (as shown in FIGS. 8G, 8H, 8J and 8K) are also encompassed within the present disclosure.

FIGS. 12A, 12B, 12C, 12D and 12E show binding of multispecific antigen-binding molecules of the present disclosure (FIGS. 1A, 1B, 4D, 4E, 4H and 4I) to CD3-expressing Jurkat cells and tumor associated antigen (TAA)-expressing cells as discussed in Example 2. “Treated” refers to protease treated to produce the corresponding cleaved molecule.

FIGS. 13A and 13B show cytotoxic activity of multispecific antigen-binding molecules of the present disclosure (FIGS. 1A and 1B) for TAA-expressing cells as discussed in Example 3, in which the external Fab binds the TAA, and the internal Fab binds human CD3. The “control” includes an external Fab that binds an unrelated/irrelevant TAA, and an internal Fab that binds human CD3.

FIGS. 14A and 14B show T-cell activation of multispecific antigen-binding molecules of the present disclosure (FIGS. 1A and 1B) in connection with TAA-expressing cells as discussed in Example 3, in which the external Fab binds the TAA, and the internal Fab binds human CD3. The “control” includes an external Fab that binds an unrelated/irrelevant TAA, and an internal Fab that binds human CD3.

FIGS. 15A, 15B, 15C and 15D show cytotoxic activity (FIGS. 15A and 15C) and T-cell activation (FIGS. 15B and 15D) of multispecific antigen-binding molecules of the present disclosure (FIGS. 4E and 4I) for TAA-expressing cells as discussed in Example 3, in which CD3 binding domain is proximal to the Fc region, and in which the CD3 binding domain binds human CD3 weakly (G20 version; FIGS. 15A and 15B) or very weakly (G5 version; FIGS. 15C and 15D). The “negative control” binds an unrelated/irrelevant TAA and CD3, and the “conventional bispecific antibody” format is a set forth in FIG. 1A of WO2021/030680, which binds the TAA and CD3. The cytotoxic and T-cell activation effects observed for the multispecific molecules at higher concentrations in FIGS. 15C and 15D is an artifact of the purification process, and the effects observed for the multispecific molecules was comparable irrespective of the CD3 binding affinity.

FIG. 16 shows cytotoxic potency of multispecific antigen-binding molecules of the present disclosure for TAA-expressing cells as discussed in Example 5, in which the length of the non-cleavable linker connecting the tandem Fabs of molecules having the structure of FIG. 4E (and FIG. 4I upon protease cleavage) is changed from (G4S)₁ to (G4S)₂ and (G4S)₃. An increase in cytotoxic potency is observed for the cleaved molecules with increasing linker length.

FIG. 17 illustrates an embodiment of a polypeptide of the present disclosure comprising tandem light chains connected by a protease cleavable linker in which the external light chain is connected at its N-terminus to a peptide mask by a protease cleavable linker.

### DETAILED DESCRIPTION

Before the present disclosure is described in further detail, it is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present disclosure, the preferred methods and materials are now described. All patents, applications and non-patent publications mentioned in this specification are incorporated herein by reference in their entireties.

### Definitions

The term “antibody” means any antigen-binding molecule or molecular complex comprising at least one complementarity determining region (CDR) that specifically binds to or interacts with a particular antigen (e.g., CD3 or a target antigen (TA)). The term “antibody” includes immunoglobulin molecules comprising four polypeptide chains, two heavy (H) chains and two light (L) chains inter-connected by disulfide bonds, as well as multimers thereof (e.g., IgM). The term “antibody” also includes immunoglobulin molecules consisting of four polypeptide chains, two heavy (H) chains and two light (L) chains inter-connected by disulfide bonds. Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-TA antibody or anti-CD3 antibody (or antigen-binding portion thereof) may be identical to the human germline sequences, or may be naturally or artificially modified. An amino acid consensus sequence may be defined based on a side-by-side analysis of two or more CDRs.

The term “antibody”, as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}- C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

In certain embodiments of the disclosure, the antibodies are human antibodies. The term “human antibody” is intended to include antibodies having variable and constant regions derived from human germline immunoglobulin sequences. The human antibodies may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The antibodies discussed herein may, in some embodiments, be recombinant human antibodies. The term “recombinant human antibody” is intended to include all human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The antibodies referenced herein may be isolated antibodies. An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” An isolated antibody also includes an antibody in situ within a recombinant cell. Isolated antibodies are antibodies that have been subjected to at least one purification or isolation step. An isolated antibody may be substantially free of other cellular material and/or chemicals.

The antibodies referenced herein may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases.

The terms “activation”, “active”, and the like in conjunction with a multispecific antigen-binding molecule of the disclosure refer to the protease-mediated enzymatic cleavage of a protease-cleavable linker that results in the unmasking of an antigen-binding domain with increased ability to bind to its target, for example through the release or separation of the antigen-binding domain from another peptide (e.g., a peptide mask, a linked Fab, or a linked Fc domain or Fc region) that sterically hinders the binding of the antigen-binding domain to its target when the protease-cleavable linker is intact. Activation may also be referred to herein as “release” of the antigen-binding domain, or the peptide mask.

The terms “anti-idiotype antibody”, “anti-idiotypic antibody” and the like refer to an antibody that recognizes the idiotype of an antigen-binding site, e.g., an antigen-binding site specific for a TCR component such as CD3. The anti-idiotype antibody is capable of specifically binding to the variable region of the antigen-binding site and thereby reducing or preventing specific binding of the antigen-binding site to its cognate antigen. When associated with a molecule that comprises the antigen-binding site, the anti-idiotype antibody can function as a masking moiety of the molecule.

The term “antigen-binding domain” refers to that portion of a multispecific molecule, or polypeptide, or a corresponding antibody that binds specifically to a predetermined antigen (e.g., CD3 or a tumor associated antigen). References to a “corresponding antibody” refer to the antibody from which the CDRs or variable regions (HCVR and LCVR) used in a multispecific molecule are derived.

The term “antigen-binding molecule” as used herein refers to a molecule (e.g., an assembly of multiple polypeptide chains) comprising one or more antigen-binding domains. The antigen-binding molecules of the disclosure can be bispecific or multispecific. The antigen-binding sites multispecific antigen-binding molecules have at least two antigen-binding domains that bind to different epitopes, which can be the same or different target molecules.

The term “associated” in the context of the molecules of the present disclosure refers to a functional relationship between two or more polypeptide chains. In particular, the term “associated” means that two or more polypeptides are associated with one another, e.g., non-covalently through molecular interactions or covalently through one or more disulfide bridges or chemical cross-linkages, so as to produce a functional multispecific antigen-binding molecule. Examples of associations that might be present in a molecule of the disclosure include (but are not limited to) associations between Fc domains in an Fc region, associations between heavy chain variable regions (HCVBR or VH) and light chain variable regions (LCVR or VL) in a Fab, and associations between CH1 and CL in a Fab.

The term “cancer” refers to a disease characterized by the uncontrolled (and often rapid) growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, adrenal gland cancer, autonomic ganglial cancer, biliary tract cancer, bone cancer, endometrial cancer, eye cancer, fallopian tube cancer, genital tract cancers, large intestinal cancer, cancer of the meninges, oesophageal cancer, peritoneal cancer, pituitary cancer, penile cancer, placental cancer, pleura cancer, salivary gland cancer, small intestinal cancer, stomach cancer, testicular cancer, thymus cancer, thyroid cancer, upper aerodigestive cancers, urinary tract cancer, vaginal cancer, vulva cancer, lymphoma, leukemia, lung cancer and the like, e.g., any TAA-positive cancers of any of the foregoing types.

The term “CD3,” as used herein, refers to an antigen which is expressed on T cells as part of the multimolecular T cell receptor (TCR) and which consists of a homodimer or heterodimer formed from the association of two of four receptor chains: CD3-epsilon, CD3-delta, CD3-zeta, and CD3-gamma. All references to proteins, polypeptides and protein fragments herein are intended to refer to the human version of the respective protein, polypeptide or protein fragment unless explicitly specified as being from a non-human species. Thus, the expression “CD3” means human CD3 unless specified as being from a non-human species, e.g., “mouse CD3,” “monkey CD3,” etc.

As used herein, the expression “cell surface-expressed” or “cell-surface molecule” means one or more protein(s) that is/are expressed on the surface of a cell in vitro or in vivo, such that at least a portion of the protein is exposed to the extracellular side of the cell membrane and is accessible to an antigen-binding portion of an antibody or an antigen-binding domain of the multispecific antigen-binding molecules discussed herein.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The term “Fab” refers to a pair of polypeptide chains, the first comprising a variable heavy (VH) domain of an antibody operably linked (typically N-terminal to) to a first constant domain (referred to herein as CH1), and the second comprising variable light (VL) domain of an antibody N-terminal operably linked (typically N-terminal) to a second constant domain (referred to herein as CL) capable of pairing with the first constant domain. In a native antibody, the VH is N-terminal to the first constant domain (CH1) of the heavy chain and the VL is N-terminal to the constant domain of the light chain (CL). The Fabs of the disclosure can be arranged according to the native orientation or include domain substitutions or swaps that facilitate correct VH and VL pairings. For example, it is possible to replace the CH1 and CL domain pair in a Fab with a CH3-domain pair to facilitate correct modified Fab-chain pairing in heterodimeric molecules. It is also possible to reverse CH1 and CL, so that the CH1 is attached to VL and CL is attached to the VH, a configuration generally known as Crossmab. The term “Fab” encompasses single chain Fabs.

The term “Fc domain” refers to a portion of the heavy chain that pairs with the corresponding portion of another heavy chain. The term “Fc region” refers to the region of antibody-based binding molecules formed by association of two heavy chain Fc domains. The two Fc domains within the Fc region may be the same or different from one another. In a native antibody the Fc domains are typically identical, but one or both Fc domains might advantageously be modified to allow for heterodimerization, e.g., via a knob-in-hole interaction.

The terms “host cell” or “recombinant host cell” refer to a cell that has been genetically-engineered, e.g., through introduction of a heterologous nucleic acid. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A host cell may carry the heterologous nucleic acid transiently, e.g., on an extrachromosomal heterologous expression vector, or stably, e.g., through integration of the heterologous nucleic acid into the host cell genome. For purposes of expressing a multispecific antigen-binding molecule or polypeptide of the disclosure, a host cell is preferably a cell line of mammalian origin or mammalian-like characteristics, such as monkey kidney cells (COS, e.g., COS-1, COS-7), HEK293), baby hamster kidney (BHK, e.g., BHK21), Chinese hamster ovary (CHO), NSO, PerC6, BSC-1, human hepatocellular carcinoma cells (e.g., Hep G2), SP2/0, HeLa, Madin-Darby bovine kidney (MDBK), myeloma and lymphoma cells, or derivatives and/or engineered variants thereof. The engineered variants include, e.g., derivatives that grow at higher density than the original cell lines and/or glycan profile modified derivatives and and/or site-specific integration site derivatives.

The term “linker” as used herein refers to a protease-cleavable linker or a non-cleavable linker unless otherwise specifically defined.

The terms “masking moiety” or “peptide mask” as used herein in relation to a multispecific antigen-binding molecule or polypeptide refers an amino acid sequence in a molecule or polypeptide that inhibits an antigen-binding domain's ability to specifically bind its target, either through a specific interaction with the antigen-binding site (e.g., where the masking moiety is an anti-idiotype antibody or fragment) or through positioning of the masking moiety or peptide mask relative to another component of the molecule or polypeptide that sterically hinders the binding of the antigen-binding domain to its target. The masking moiety or peptide mask are arranged in the molecule or polypeptide such that cleavage of a protease cleavable linker reduces the inhibition of the antigen-binding domain's interaction with its target, either through the generation of a molecule or polypeptide that lacks the masking moiety or a molecule or polypeptide in which spatial constraints on the antigen-binding domain's ability to interact with its target are alleviated. The terms “masking moiety” or “peptide mask” when used in relation to an antigen binding-molecule more generally refers to an amino acid sequence in the antigen-binding molecule proprotein that inhibits the ability of an antigen binding domain in the molecule or polypeptide to specifically bind its target.

A “multimerization domain” or “multimerizing domain” is any macromolecule that has the ability to associate (covalently or non-covalently) with a second macromolecule of the same or similar structure or constitution. For example, a multimerization domain may be a polypeptide comprising an immunoglobulin C_{H}3 domain. A non-limiting example of a multimerization domain is an Fc portion of an immunoglobulin, e.g., an Fc domain of an IgG selected from the isotypes IgG1, IgG2, IgG3, and IgG4, as well as any allotype within each isotype group. In certain embodiments, the multimerization domain is an Fc fragment or an amino acid sequence of 1 to about 200 amino acids in length containing at least one cysteine residue. In other embodiments, the multimerization domain is a cysteine residue or a short cysteine-containing peptide. Other multimerization domains include peptides or polypeptides comprising or consisting of a leucine zipper, a helix-loop motif, or a coiled-coil motif. In some embodiments, the multimerizing domain is an immunoglobulin Fc domain and the multispecific antigen-binding molecules of the present disclosure are formed by association of two such Fc domains via interchain disulfide bonding as in a conventional antibody. In any embodiments discussed herein, the Fc domain(s) or Fc region may lack the C-terminal lysine residue(s) that would ordinarily be present at the C-terminus of a mature immunoglobulin molecule (e.g., of a human IgG1, IgG2, IgG3 or IgG4 Fc domain). Such embodiments reduce the potential for a serine protease site at the C-terminus of the Fc domain(s).

A “non-cleavable linker” refers to a peptide whose amino acid sequence lacks a substrate sequence for a protease.

The terms “nucleic acid” or “polynucleotide” refer to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.

The terms “polypeptide”, “peptide” and “protein” are used interchangeably herein to refer to a polymer of amino acid residues.

The term “protease” as used herein refers to any enzyme that catalyzes hydrolysis of a peptide bond. Generally, the proteases useful in the present disclosure, e.g., the proteases described herein, recognize and cleave a specific sequence motif, e.g., a substrate as described herein. Preferably, the proteases are expressed at higher levels in cancer tissues as compared to normal tissues.

As used herein, the term “protease cleavable linker” refers to a peptide whose amino acid sequence contains one or more (e.g., two or three) substrate sequences for one or more proteases.

The term “recombinant,” as used herein, is intended to include all molecules that are prepared, expressed, created or isolated by recombinant means, such as multispecific molecules (e.g. bispecific molecules) expressed using a recombinant expression vector transfected into a host cell, multispecific molecules (e.g., bispecific molecules) isolated from an animal (e.g., a mouse) that is transgenic for human immunoglobulin genes (see e.g., Taylor et al. (1992) Nucl. Acids Res. 20:6287-6295) or multispecific molecules prepared, expressed, created or isolated by any other means that involves splicing of human immunoglobulin and/or MHC gene sequences to other DNA sequences. Such recombinant multispecific molecules can include antigen-binding domains having variable and constant regions derived from human germline immunoglobulin sequences.

As used herein, the term “spacer” refers to a peptide, the amino acid sequence of which is not a substrate for a protease, incorporated into a linker containing a substrate. A spacer can be used to separate the substrate from other domains in a molecule, e.g., antigen-binding domains. In some aspects, residues in the spacer minimize aminopeptidase and/or exopeptidase action to prevent cleavage of N-terminal amino acids.

The term “specifically (or selectively) binds” to an antigen or an epitope refers to a binding reaction that is determinative of the presence of a cognate antigen or an epitope in a heterogeneous population of proteins and other molecules. The binding reaction can be but need not be mediated by an antibody or antibody fragment. The term “specifically binds” does not exclude cross-species reactivity. For example, an antigen-binding site (e.g., an antigen-binding fragment of an antibody) that “specifically binds” to an antigen from one species may also “specifically bind” to that antigen in one or more other species. Thus, such cross-species reactivity does not itself alter the classification of an antigen-binding site as a “specific” binder. In certain embodiments, an antigen-binding domain of the disclosure that specifically binds to a human antigen has cross-species reactivity with one or more non-human mammalian species, e.g., a primate species (including but not limited to one or more of Macaca fascicularis, Macaca mulatta, and Macaca nemestrina) or a rodent species, e.g., Mus musculus.

The term “subject” includes human and non-human animals. Non-human animals include all vertebrates, e.g., mammals and non-mammals, such as non-human primates, sheep, dog, cow, chickens, amphibians, and reptiles. In preferred embodiments, the subject is human.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445, herein incorporated by reference. A “moderately conservative” replacement is any change having a nonnegative value in the PAM250 log-likelihood matrix.

The term “T cell” refers to immune cells expressing CD3, including CD4+ cells (helper T cells), CD8+ cells (cytotoxic T cells), regulatory T cells (Tregs), and tumor infiltrating lymphocytes.

The term “T-cell antigen” or “TCA” refers to a molecule (typically a protein, carbohydrate, lipid or some combination thereof) that is expressed on the surface of a T-lymphocyte and is useful for the preferential targeting of a pharmacological agent to a particular site. In some embodiments, the site is cancer tissue and/or the T-cell antigen is a tumor reactive lymphocyte antigen, a cell surface molecule of tumor or viral lymphocytes, or a checkpoint inhibitor expressed on a T-lymphocyte. A “co-stimulatory molecule” refers to a protein expressed by a T cell that binds a cognate ligand or receptor (e.g., on an antigen-presenting cell) to provide a stimulatory signal, which, in combination with the primary signal provided by engagement of the T cell's TCR with a peptide/MHC, stimulates the activity of the T cell. Stimulation of a T cell can include activation, proliferation and/or survival of the T cell.

The term “tumor” is used interchangeably with the term “cancer” herein, e.g., both terms encompass solid and liquid, e.g., diffuse or circulating, tumors. As used herein, the term “cancer” or “tumor” includes premalignant, as well as malignant cancers and tumors.

The term “tumor-associated antigen” or “TAA” refers to a molecule (typically a protein, carbohydrate, lipid or some combination thereof) that is expressed on the surface of a cancer cell, either entirely or as a fragment (e.g., MHC/peptide), and which is useful for the preferential targeting of a pharmacological agent to the cancer cell. In some embodiments, a TAA is a marker expressed by both normal cells and cancer cells, e.g., a lineage marker. In some embodiments, a TAA is a cell surface molecule that is overexpressed in a cancer cell in comparison to a normal cell, for instance, 1-fold over expression, 2-fold overexpression, 3-fold overexpression or more in comparison to a normal cell. In some embodiments, a TAA is a cell surface molecule that is inappropriately synthesized in the cancer cell, for instance, a molecule that contains deletions, additions or mutations in comparison to the molecule expressed on a normal cell. In some embodiments, a TAA will be expressed exclusively on the cell surface of a cancer cell, entirely or as a fragment (e.g., MHC/peptide), and not synthesized or expressed on the surface of a normal cell. Accordingly, the term “TAA” encompasses antigens that are specific to cancer cells, sometimes known in the art as tumor-specific antigens.

As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more molecules or polypeptides of the disclosure. In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

The term “universal light chain” or “ULC” as used herein refers to a light chain variable region (VL) that can pair with more than one heavy chain variable region (VL). In the context of an antigen-binding domain, the term “universal light chain” or “ULC” refers to a light chain polypeptide capable of pairing with the heavy chain region of the antigen-binding domain and also capable of pairing with other heavy chain regions. ULCs can also include constant domains, e.g., a CL domain of an antibody. Universal light chains are also known as “common light chains”.

The terms “vector” and “expression vector” include, but are not limited to, a viral vector, a plasmid, an RNA vector or a linear or circular DNA or RNA molecule which may consist of chromosomal, non-chromosomal, semi-synthetic or synthetic nucleic acids. In some cases, the vectors are those capable of autonomous replication (episomal vector) and/or expression of nucleic acids to which they are linked (expression vectors). Large numbers of suitable vectors are known to those of skill in the art and are commercially available. Viral vectors include retrovirus, adenovirus, parvovirus (e.g., adenoassociated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, and lentivirus.

### Multispecific Antigen-Binding Molecules

The present disclosure relates to multispecific antigen-binding molecules and polypeptides (e.g., as proproteins comprising cleavable linkers and/or masking moieties, and their activated forms following cleavage of the linkers). In the proprotein form, the molecules and polypeptides of the present disclosure comprise one or more antigen-binding domains that are blocked from binding their target antigen by the presence of a linked molecule, which may be a masking moiety, an Fc domain or Fc region, or another antigen-binding domain. The molecules and polypeptides include a protease-cleavable linker, arranged so that the masking moiety or other linked molecule diminishes or blocks the antigen-binding domain from binding to its target, and configured such that upon encountering a protease, e.g., a protease that is overexpressed in the tumor environment, the masking moiety is cleaved and a binding molecule is produced having enhanced target binding relative to the non-cleaved form of the molecule. Typically, the masking moiety of an antigen-binding molecule proprotein masks the antigen-binding domain via steric hindrance (e.g., an Fc domain masking moiety) and/or via binding of a targeting moiety to the antigen-binding site (e.g., an anti-idiotype binding moiety).

In some cases, the multispecific antigen-binding molecules (e.g., bispecific) of the present disclosure comprise (a) a first immunoglobulin antigen-binding fragment (Fab) comprising a first polypeptide comprising, from N-terminus to C-terminus, a light chain variable region (LCVR) and an immunoglobulin light chain constant region (CL), and a second polypeptide comprising, from N-terminus to C-terminus, a heavy chain variable region (HCVR) and an immunoglobulin CH1 heavy chain constant region (CH1), wherein the first polypeptide and the second polypeptide are connected by a disulfide bond; (b) a second immunoglobulin Fab comprising a first polypeptide comprising, from N-terminus to C-terminus, a LCVR and a CL, and a second polypeptide comprising, from N-terminus to C-terminus, a HCVR and a CH1; and (c) an immunoglobulin Fc region comprising a pair of polypeptides, each comprising a hinge region, an immunoglobulin CH2 heavy chain constant region (CH2) and an immunoglobulin CH3 heavy chain constant region (CH3), wherein the pair of polypeptides are connected by a disulfide bond or a non-cleavable linker, optionally via a disulfide bond in the hinge region, wherein the first immunoglobulin Fab and the second immunoglobulin Fab are connected by a first linker between the C-terminus of the CL of the first immunoglobulin Fab and the N-terminus of the LCVR of the second immunoglobulin Fab, and by a second linker between the C-terminus of the CH1 of the first immunoglobulin Fab and the N-terminus of the HCVR of the second immunoglobulin Fab, and wherein the first linker or the second linker, but not both, is a protease cleavable linker, and wherein the first immunoglobulin Fab or the second immunoglobulin Fab, but not both, is connected to the immunoglobulin Fc region by a third linker that is a protease cleavable linker.

In some cases, the disulfide bond connecting the first polypeptide and the second polypeptide of the first immunoglobulin Fab may be a disulfide bond between the CL to the CH1. In some cases, the disulfide bond connecting the first polypeptide and the second polypeptide of the second immunoglobulin Fab may be a disulfide bond between the CL to the CH1. In some cases, the disulfide bond connecting the first polypeptide and the second polypeptide of the second immunoglobulin Fab is a disulfide bond between the LCVR and the HCVR. In some cases, the LCVR of the second immunoglobulin Fab comprises a cysteine mutation at residue 100 (Kabat numbering), and the HCVR of the second immunoglobulin Fab comprises a cysteine mutation at residue 44 (Kabat numbering).

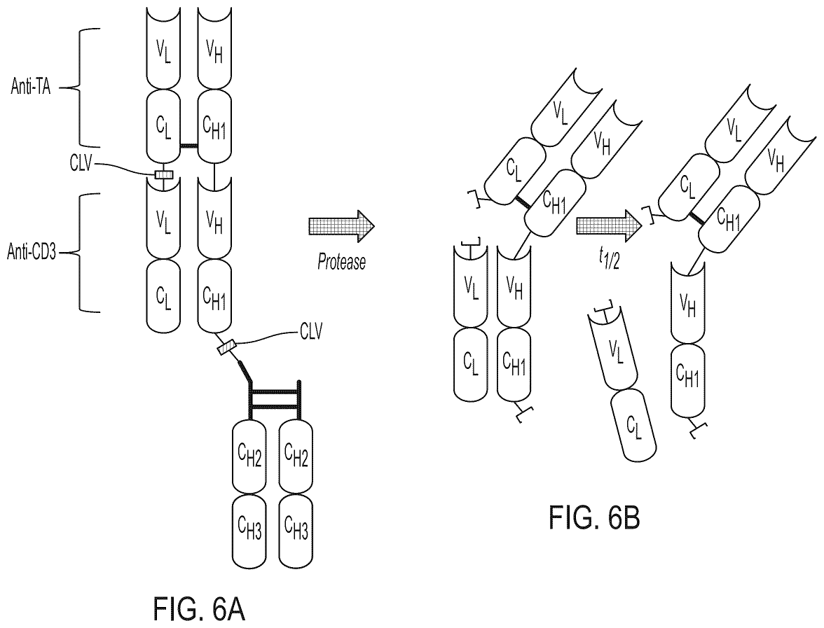

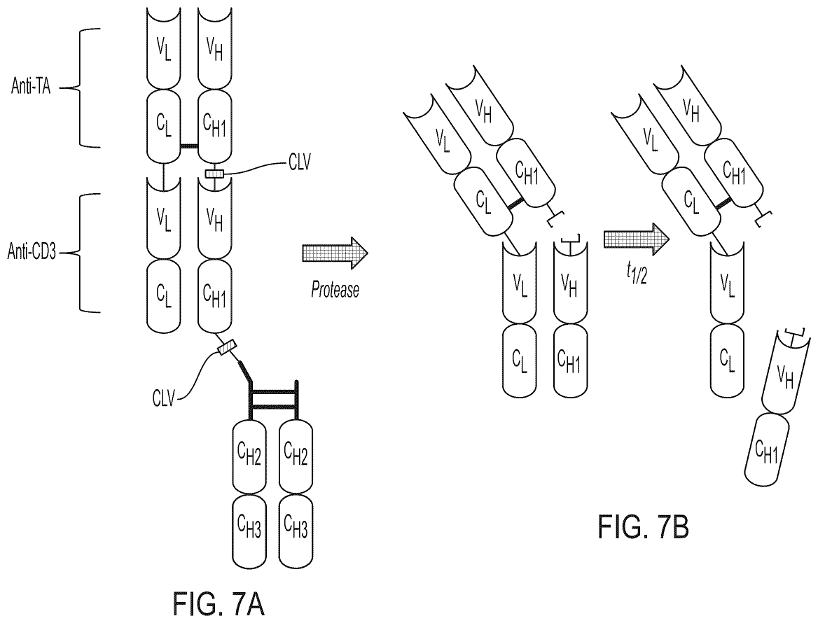

In some cases, the first polypeptide and the second polypeptide of the second immunoglobulin Fab are not connected by a disulfide bond. In such cases, as illustrated in the embodiments of FIGS. 6A and 7A, activation of the molecule produces a cleaved product in which the light chain and heavy chain portions of the second immunoglobulin Fab are connected only by non-covalent bonding, which may lead to a loss of function of this second antigen-binding domain following some period of time (e.g., a half-life), thereby providing a safety feature to prevent long term availability of this antigen-binding domain (e.g., a CD3 binding domain) from remaining active indefinitely.

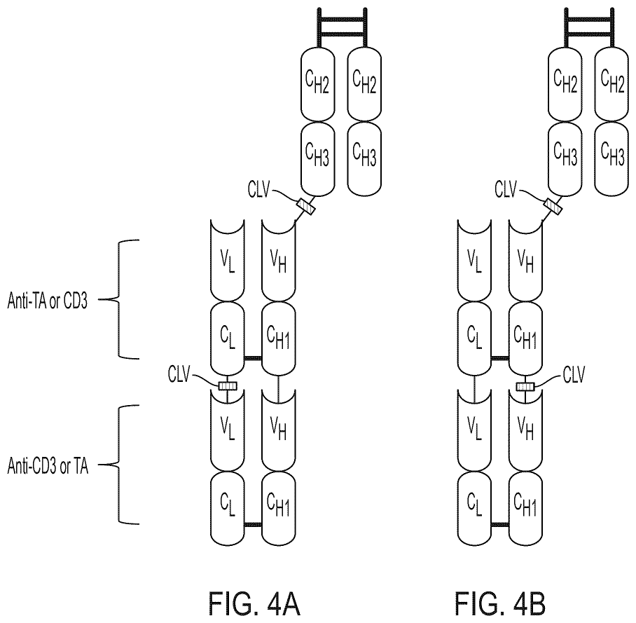

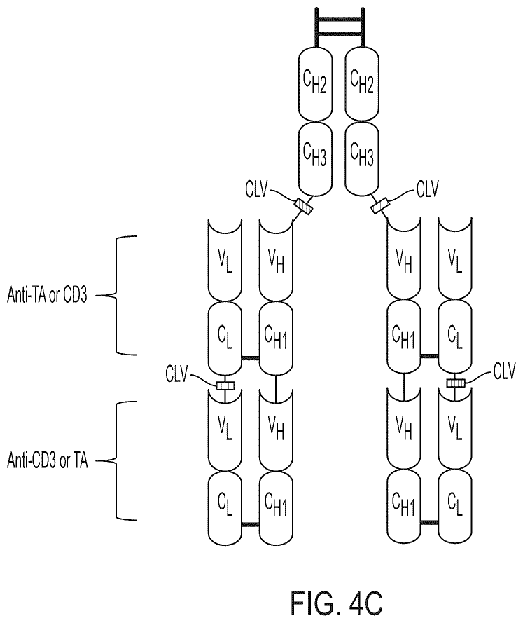

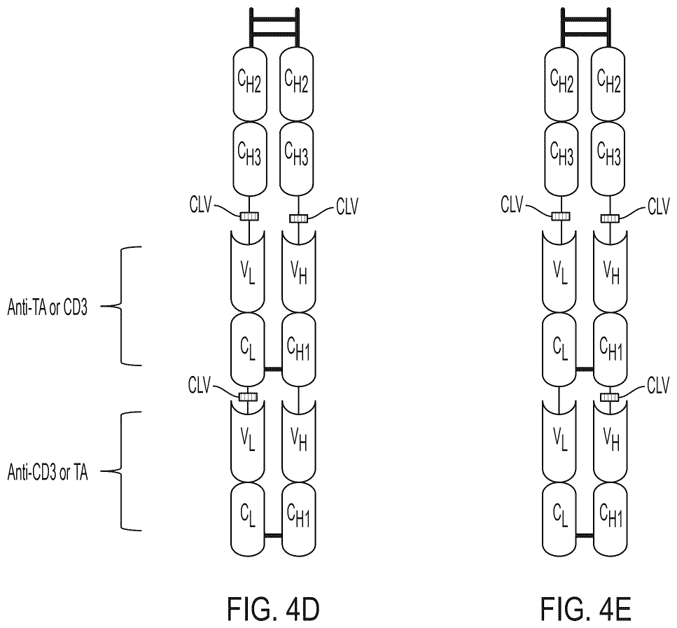

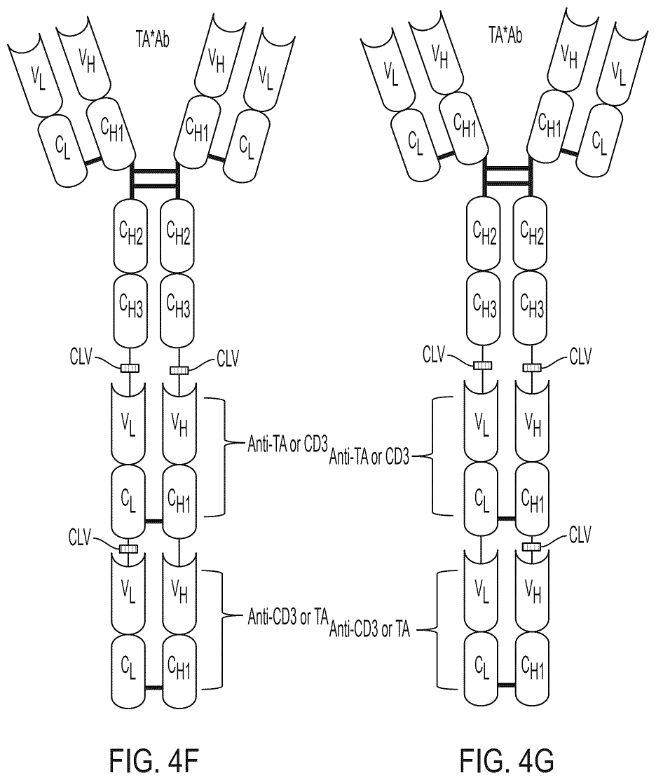

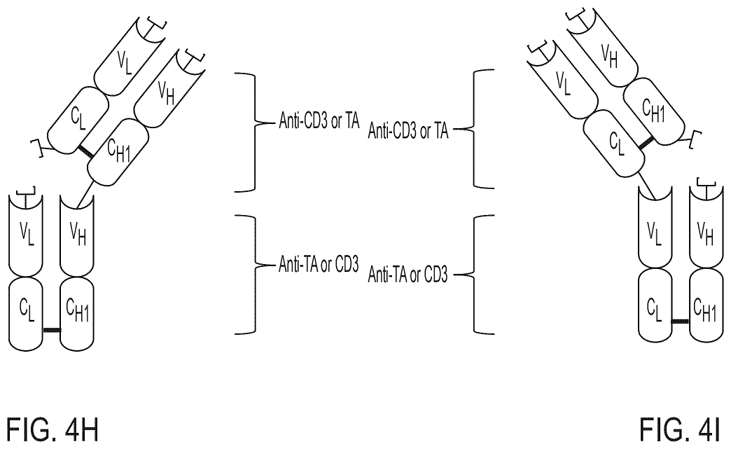

As can be seen in the various embodiments illustrated in the figures, either of the linkers connecting the tandem Fabs can be a cleavable linker (e.g., a protease cleavable linker). In some cases, the linker between the light chain portions of the Fabs is a protease cleavable linker (e.g., FIG. 4A or FIG. 4D or FIG. 4F), and in some cases the linker between the heavy chain portions of the Fabs is a protease cleavable linker (e.g., FIG. 4B or FIG. 4E or FIG. 4G).

The linker connecting one or more Fabs to the multimerizing domain (e.g., the Fc domain, the Fc region, or the hinge region) may be connected to the light chain portion of a Fab or the heavy chain portion of a Fab. Thus, in some cases, the linker connects the C-terminus of the CH1 of the second immunoglobulin Fab to the hinge region of the immunoglobulin Fc region, and in some cases, the linker connects the C-terminus of the CL of the second immunoglobulin Fab to the hinge region of the immunoglobulin Fc region. Similar attachments options are available for the alternative format of the molecule shown in, e.g., FIG. 4A.

In some cases, the multispecific antigen-binding molecules (e.g., bispecific, trispecific, or tetraspecific) of the present disclosure comprise (a) a first immunoglobulin antigen-binding fragment (Fab) comprising a first polypeptide comprising, from N-terminus to C-terminus, a light chain variable region (LCVR) and an immunoglobulin light chain constant region (CL), and a second polypeptide comprising, from N-terminus to C-terminus, a heavy chain variable region (HCVR) and an immunoglobulin CH1 heavy chain constant region (CH1), wherein the first polypeptide and the second polypeptide are connected by a disulfide bond; (b) a second immunoglobulin Fab comprising a first polypeptide comprising, from N-terminus to C-terminus, a LCVR and a CL, and a second polypeptide comprising, from N-terminus to C-terminus, a HCVR and a CH1, wherein the first immunoglobulin Fab and the second immunoglobulin Fab are connected by a first linker between the C-terminus of the CL of the first immunoglobulin Fab and the N-terminus of the LCVR of the second immunoglobulin Fab, and by a second linker between the C-terminus of the CH1 of the first immunoglobulin Fab and the N-terminus of the HCVR of the second immunoglobulin Fab; (c) a third immunoglobulin Fab comprising a first polypeptide comprising, from N-terminus to C-terminus, a LCVR and a CL, and a second polypeptide comprising, from N-terminus to C-terminus, a HCVR and a CH1; (d) a fourth immunoglobulin Fab comprising a first polypeptide comprising, from N-terminus to C-terminus, a LCVR and a CL, and a second polypeptide comprising, from N-terminus to C-terminus, a HCVR and a CH1, wherein the third immunoglobulin Fab and the fourth immunoglobulin Fab are connected by a fifth linker between the C-terminus of the CL of the third immunoglobulin Fab and the N-terminus of the LCVR of the fourth immunoglobulin Fab, and by a sixth linker between the C-terminus of the CH1 of the third immunoglobulin Fab and the N-terminus of the HCVR of the fourth immunoglobulin Fab; and (e) an immunoglobulin Fc region comprising a pair of polypeptides, each comprising a hinge region, an immunoglobulin CH2 heavy chain constant region (CH2) and an immunoglobulin CH3 heavy chain constant region (CH3), wherein the pair of polypeptides are connected by a disulfide bond in the hinge region, and wherein the first linker or the second linker, but not both, is a protease cleavable linker, and wherein the fifth linker or the sixth linker, but not both, is a protease cleavable linker, and wherein the first immunoglobulin Fab or the second immunoglobulin Fab, but not both, is connected to the immunoglobulin Fc region by a third linker that is a protease cleavable linker, and wherein the third immunoglobulin Fab or the fourth immunoglobulin Fab, but not both, is connected to the immunoglobulin Fc region by a seventh linker that is a protease cleavable linker, and wherein (i) if the first immunoglobulin Fab is connected to the immunoglobulin Fc region, then the third immunoglobulin Fab is also connected to the immunoglobulin Fc region, or (ii) if the second immunoglobulin Fab is connected to the immunoglobulin Fc region, then the fourth immunoglobulin Fab is also connected to the immunoglobulin Fc region.

In some cases, the multispecific antigen-binding molecules (e.g., bispecific) of the present disclosure comprise (a) a first immunoglobulin antigen-binding fragment (Fab) comprising a first polypeptide comprising, from N-terminus to C-terminus, a light chain variable region (LCVR) and an immunoglobulin light chain constant region (CL), and a second polypeptide comprising, from N-terminus to C-terminus, a heavy chain variable region (HCVR) and an immunoglobulin CH1 heavy chain constant region (CH1), wherein the first polypeptide and the second polypeptide are connected by a disulfide bond; (b) a second immunoglobulin Fab comprising a first polypeptide comprising, from N-terminus to C-terminus, a LCVR and a CL, and a second polypeptide comprising, from N-terminus to C-terminus, a HCVR and a CH1; and (c) an immunoglobulin Fc region comprising a first polypeptide and a second polypeptide, each polypeptide comprising an immunoglobulin CH2 heavy chain constant region (CH2) and an immunoglobulin CH3 heavy chain constant region (CH3), wherein the first and second polypeptides are connected by a disulfide bond or a non-cleavable linker, wherein the first immunoglobulin Fab and the second immunoglobulin Fab are connected by a first linker between the C-terminus of the CL of the first immunoglobulin Fab and the N-terminus of the LCVR of the second immunoglobulin Fab, and by a second linker between the C-terminus of the CH1 of the first immunoglobulin Fab and the N-terminus of the HCVR of the second immunoglobulin Fab, and wherein the first linker is a non-cleavable linker and the second linker is a protease cleavable linker, and wherein the first immunoglobulin Fab is connected to the immunoglobulin Fc region by a third linker that is a protease cleavable linker between the N-terminus of the LCVR of the first immunoglobulin Fab and the C-terminus of the CH3 of the first polypeptide of the immunoglobulin Fc region, and by a fourth linker that is a protease cleavable linker between the N-terminus of the HCVR of the first immunoglobulin Fab and the C-terminus of the CH3 of the second polypeptide of the immunoglobulin Fc region.

The present disclosure also relates to multispecific antigen-binding molecules comprising: (a) a first immunoglobulin antigen-binding fragment (Fab) comprising: a first polypeptide comprising, from N-terminus to C-terminus, a peptide mask, a protease cleavable linker, a light chain variable region (LCVR) and an immunoglobulin light chain constant region (CL); and a second polypeptide comprising, from N-terminus to C-terminus, a peptide mask, a protease cleavable linker, a heavy chain variable region (HCVR) and an immunoglobulin CH1 heavy chain constant region (CH1), wherein the first polypeptide and the second polypeptide are connected by a disulfide bond; (b) a second immunoglobulin Fab comprising: a first polypeptide comprising, from N-terminus to C-terminus, a peptide mask, a protease cleavable linker, a LCVR and a CL; and a second polypeptide comprising, from N-terminus to C-terminus, a HCVR and a CH1, wherein the first polypeptide and the second polypeptide are connected by a disulfide bond; and (c) an immunoglobulin Fc region comprising a pair of polypeptides, each comprising a hinge region, an immunoglobulin CH2 heavy chain constant region (CH2) and an immunoglobulin CH3 heavy chain constant region (CH3), wherein the pair of polypeptides are connected by a disulfide bond or a non-cleavable linker, wherein the first immunoglobulin Fab and the second immunoglobulin Fab are connected by a non-cleavable linker between the C-terminus of the CH1 of the first immunoglobulin Fab and the N-terminus of the HCVR of the second immunoglobulin Fab, and wherein the first immunoglobulin Fab is connected to the immunoglobulin Fc region by a non-cleavable linker between the peptide mask of the second polypeptide and a CH3 of the immunoglobulin Fc region.

The present disclosure also relates to multispecific antigen-binding molecules comprising: (a) a first immunoglobulin antigen-binding fragment (Fab) comprising: a first polypeptide comprising, from N-terminus to C-terminus, a light chain variable region (LCVR) and an immunoglobulin light chain constant region (CL); and a second polypeptide comprising, from N-terminus to C-terminus, a peptide mask, a protease cleavable linker, a heavy chain variable region (HCVR) and an immunoglobulin CH1 heavy chain constant region (CH1), wherein the first polypeptide and the second polypeptide are connected by a disulfide bond; (b) a second immunoglobulin Fab comprising: a first polypeptide comprising, from N-terminus to C-terminus, a LCVR and a CL; and a second polypeptide comprising, from N-terminus to C-terminus, a HCVR and a CH1, wherein the first polypeptide and the second polypeptide are connected by a disulfide bond; and (c) an immunoglobulin Fc region comprising a pair of polypeptides, each comprising a hinge region, an immunoglobulin CH2 heavy chain constant region (CH2) and an immunoglobulin CH3 heavy chain constant region (CH3), wherein the pair of polypeptides are connected by a disulfide bond or a non-cleavable linker, wherein the first immunoglobulin Fab and the second immunoglobulin Fab are connected by a non-cleavable linker between the C-terminus of the CH1 of the first immunoglobulin Fab and the N-terminus of the HCVR of the second immunoglobulin Fab, and wherein the first immunoglobulin Fab is connected to the immunoglobulin Fc region by a non-cleavable linker between the peptide mask of the second polypeptide and a CH3 of the immunoglobulin Fc region.

The present disclosure also relates to multispecific antigen-binding molecules comprising: (a) a first immunoglobulin antigen-binding fragment (Fab) comprising: a first polypeptide comprising, from N-terminus to C-terminus, a peptide mask, a protease cleavable linker, a light chain variable region (LCVR) and an immunoglobulin light chain constant region (CL); and a second polypeptide comprising, from N-terminus to C-terminus, a heavy chain variable region (HCVR) and an immunoglobulin CH1 heavy chain constant region (CH1), wherein the first polypeptide and the second polypeptide are connected by a disulfide bond; (b) a second immunoglobulin Fab comprising: a first polypeptide comprising, from N-terminus to C-terminus, a peptide mask, a protease cleavable linker, a LCVR and a CL; and a second polypeptide comprising, from N-terminus to C-terminus, a HCVR and a CH1, wherein the first polypeptide and the second polypeptide are connected by a disulfide bond; and (c) an immunoglobulin Fc region comprising a pair of polypeptides, each comprising a hinge region, an immunoglobulin CH2 heavy chain constant region (CH2) and an immunoglobulin CH3 heavy chain constant region (CH3), wherein the pair of polypeptides are connected by a disulfide bond or a non-cleavable linker, wherein the first immunoglobulin Fab and the second immunoglobulin Fab are connected by a non-cleavable linker between the C-terminus of the CH1 of the first immunoglobulin Fab and the N-terminus of the HCVR of the second immunoglobulin Fab, and

wherein the first immunoglobulin Fab is connected to the immunoglobulin Fc region by a protease cleavable linker between the HCVR of the second polypeptide and a CH3 of the immunoglobulin Fc region.

In some embodiments, the immunoglobulin Fc region is an immunoglobulin Fc region of an antibody, wherein the antibody comprises two Fabs, each comprising a first polypeptide comprising, from N-terminus to C-terminus, a peptide mask, a protease cleavable linker, a LCVR and a CL, and a second polypeptide comprising a HCVR and a CH1, wherein the first and second polypeptide are connected by a disulfide bond, and the wherein the CH1 of each of the two Fabs is connected to the hinge region of the immunoglobulin Fc region.

In some embodiments, the first immunoglobulin Fab binds a T-cell antigen, and the second immunoglobulin Fab binds a target antigen. In some cases, the first immunoglobulin Fab binds a target antigen, and the second immunoglobulin Fab binds a T-cell antigen. In some embodiments, the target antigen is a tumor-associated antigen. In some embodiments, the T-cell antigen is CD3.

The present disclosure also relates to multispecific antigen-binding molecules comprising: (a) a first immunoglobulin antigen-binding fragment (Fab) comprising: a first polypeptide comprising, from N-terminus to C-terminus, a peptide mask, a protease cleavable linker, a light chain variable region (LCVR) and an immunoglobulin light chain constant region (CL); and a second polypeptide comprising, from N-terminus to C-terminus, a peptide mask, a protease cleavable linker, a heavy chain variable region (HCVR) and an immunoglobulin CH1 heavy chain constant region (CH1), wherein the first polypeptide and the second polypeptide are connected by a disulfide bond; (b) a second immunoglobulin Fab comprising: a first polypeptide comprising, from N-terminus to C-terminus, a peptide mask, a protease cleavable linker, a LCVR and a CL; and a second polypeptide comprising, from N-terminus to C-terminus, a HCVR and a CH1, wherein the first polypeptide and the second polypeptide are connected by a disulfide bond; (c) a third immunoglobulin Fab comprising: a first polypeptide comprising, from N-terminus to C-terminus, a peptide mask, a protease cleavable linker, a LCVR and a CL; and a second polypeptide comprising, from N-terminus to C-terminus, a peptide mask, a protease cleavable linker, a HCVR and a CH1, wherein the first polypeptide and the second polypeptide are connected by a disulfide bond; (d) a fourth immunoglobulin Fab comprising: a first polypeptide comprising, from N-terminus to C-terminus, a peptide mask, a protease cleavable linker, a LCVR and a CL; and a second polypeptide comprising, from N-terminus to C-terminus, a HCVR and a CH1, wherein the first polypeptide and the second polypeptide are connected by a disulfide bond; and (e) an immunoglobulin Fc region comprising a pair of polypeptides, each comprising a hinge region, an immunoglobulin CH2 heavy chain constant region (CH2) and an immunoglobulin CH3 heavy chain constant region (CH3), wherein the pair of polypeptides are connected by a disulfide bond or a non-cleavable linker, wherein the first immunoglobulin Fab and the second immunoglobulin Fab are connected by a non-cleavable linker between the C-terminus of the CH1 of the first immunoglobulin Fab and the N-terminus of the HCVR of the second immunoglobulin Fab, wherein the third immunoglobulin Fab and the fourth immunoglobulin Fab are connected by a non-cleavable linker between the C-terminus of the CH1 of the third immunoglobulin Fab and the N-terminus of the HCVR of the fourth immunoglobulin Fab, wherein the first immunoglobulin Fab is connected to the immunoglobulin Fc region by a non-cleavable linker between the peptide mask of the second polypeptide and a CH3 of the immunoglobulin Fc region, and wherein the third immunoglobulin Fab is connected to the immunoglobulin Fc region by a non-cleavable linker between the peptide mask of the second polypeptide and a CH3 of the immunoglobulin Fc region.

The present disclosure also relates to multispecific antigen-binding molecules comprising: (a) a first immunoglobulin antigen-binding fragment (Fab) comprising: a first polypeptide comprising, from N-terminus to C-terminus, a light chain variable region (LCVR) and an immunoglobulin light chain constant region (CL); and a second polypeptide comprising, from N-terminus to C-terminus, a peptide mask, a protease cleavable linker, a heavy chain variable region (HCVR) and an immunoglobulin CH1 heavy chain constant region (CH1), wherein the first polypeptide and the second polypeptide are connected by a disulfide bond; (b) a second immunoglobulin Fab comprising: a first polypeptide comprising, from N-terminus to C-terminus, a LCVR and a CL; and a second polypeptide comprising, from N-terminus to C-terminus, a HCVR and a CH1, wherein the first polypeptide and the second polypeptide are connected by a disulfide bond; (c) a third immunoglobulin Fab comprising: a first polypeptide comprising, from N-terminus to C-terminus, a LCVR and a CL; and a second polypeptide comprising, from N-terminus to C-terminus, a peptide mask, a protease cleavable linker, a HCVR and a CH1, wherein the first polypeptide and the second polypeptide are connected by a disulfide bond; (d) a fourth immunoglobulin Fab comprising: a first polypeptide comprising, from N-terminus to C-terminus, a LCVR and a CL; and a second polypeptide comprising, from N-terminus to C-terminus, a HCVR and a CH1, wherein the first polypeptide and the second polypeptide are connected by a disulfide bond; and (e) an immunoglobulin Fc region comprising a pair of polypeptides, each comprising a hinge region, an immunoglobulin CH2 heavy chain constant region (CH2) and an immunoglobulin CH3 heavy chain constant region (CH3), wherein the pair of polypeptides are connected by a disulfide bond or a non-cleavable linker, wherein the first immunoglobulin Fab and the second immunoglobulin Fab are connected by a non-cleavable linker between the C-terminus of the CH1 of the first immunoglobulin Fab and the N-terminus of the HCVR of the second immunoglobulin Fab, wherein the third immunoglobulin Fab and the fourth immunoglobulin Fab are connected by a non-cleavable linker between the C-terminus of the CH1 of the third immunoglobulin Fab and the N-terminus of the HCVR of the fourth immunoglobulin Fab, wherein the first immunoglobulin Fab is connected to the immunoglobulin Fc region by a non-cleavable linker between the peptide mask of the second polypeptide and a CH3 of the immunoglobulin Fc region, and wherein the third immunoglobulin Fab is connected to the immunoglobulin Fc region by a non-cleavable linker between the peptide mask of the second polypeptide and a CH3 of the immunoglobulin Fc region.

The present disclosure also relates to multispecific antigen-binding molecules comprising: (a) a first immunoglobulin antigen-binding fragment (Fab) comprising: a first polypeptide comprising, from N-terminus to C-terminus, a peptide mask, a protease cleavable linker, a light chain variable region (LCVR) and an immunoglobulin light chain constant region (CL); and a second polypeptide comprising, from N-terminus to C-terminus, a heavy chain variable region (HCVR) and an immunoglobulin CH1 heavy chain constant region (CH1), wherein the first polypeptide and the second polypeptide are connected by a disulfide bond; (b) a second immunoglobulin Fab comprising: a first polypeptide comprising, from N-terminus to C-terminus, a peptide mask, a protease cleavable linker, a LCVR and a CL; and a second polypeptide comprising, from N-terminus to C-terminus, a HCVR and a CH1, wherein the first polypeptide and the second polypeptide are connected by a disulfide bond; (c) a third immunoglobulin Fab comprising: a first polypeptide comprising, from N-terminus to C-terminus, a peptide mask, a protease cleavable linker, a LCVR and a CL; and a second polypeptide comprising, from N-terminus to C-terminus, a HCVR and a CH1, wherein the first polypeptide and the second polypeptide are connected by a disulfide bond; (d) a fourth immunoglobulin Fab comprising: a first polypeptide comprising, from N-terminus to C-terminus, a peptide mask, a protease cleavable linker, a LCVR and a CL; and a second polypeptide comprising, from N-terminus to C-terminus, a HCVR and a CH1, wherein the first polypeptide and the second polypeptide are connected by a disulfide bond; and (e) an immunoglobulin Fc region comprising a pair of polypeptides, each comprising a hinge region, an immunoglobulin CH2 heavy chain constant region (CH2) and an immunoglobulin CH3 heavy chain constant region (CH3), wherein the pair of polypeptides are connected by a disulfide bond or a non-cleavable linker, wherein the first immunoglobulin Fab and the second immunoglobulin Fab are connected by a non-cleavable linker between the C-terminus of the CH1 of the first immunoglobulin Fab and the N-terminus of the HCVR of the second immunoglobulin Fab, wherein the third immunoglobulin Fab and the fourth immunoglobulin Fab are connected by a non-cleavable linker between the C-terminus of the CH1 of the third immunoglobulin Fab and the N-terminus of the HCVR of the fourth immunoglobulin Fab, wherein the first immunoglobulin Fab is connected to the immunoglobulin Fc region by a protease cleavable linker between the HCVR of the second polypeptide and a CH3 of the immunoglobulin Fc region, and wherein the third immunoglobulin Fab is connected to the immunoglobulin Fc region by a protease cleavable linker between the HCVR of the second polypeptide and a CH3 of the immunoglobulin Fc region.

In some embodiments, the immunoglobulin Fc region is an immunoglobulin Fc region of an antibody, wherein the antibody comprises two Fabs, each comprising a first polypeptide comprising, from N-terminus to C-terminus, a peptide mask, a protease cleavable linker, a LCVR and a CL, and a second polypeptide comprising a HCVR and a CH1, wherein the first and second polypeptide are connected by a disulfide bond, and the wherein the CH1 of each of the two Fabs is connected to the hinge region of the immunoglobulin Fc region.

In some embodiments, the first immunoglobulin Fab and the third immunoglobulin Fab bind a T-cell antigen, and the second immunoglobulin Fab and the fourth immunoglobulin Fab bind a target antigen. In some embodiments, the first immunoglobulin Fab and the third immunoglobulin Fab bind a target antigen, and the second immunoglobulin Fab and the fourth immunoglobulin Fab bind a T-cell antigen. In some embodiments, the target antigen is a tumor-associated antigen. In some embodiments, the T-cell antigen is a CD3.

In various embodiments, each polypeptide comprising (i) a peptide mask, a protease cleavable linker, a LCVR and a CL, or (ii) a LCVR and a CL, is identical (e.g., the LCVR may be a ULC that can pair with a variety of HCVRs to produce an antigen-binding domain, wherein the antigen-binding specificity is determined by the HCVR). In some cases, each peptide mask is an anti-idiotype antigen-binding molecule that binds either a target antigen binding domain of a Fab or a T-cell antigen binding domain of a Fab, but not both.

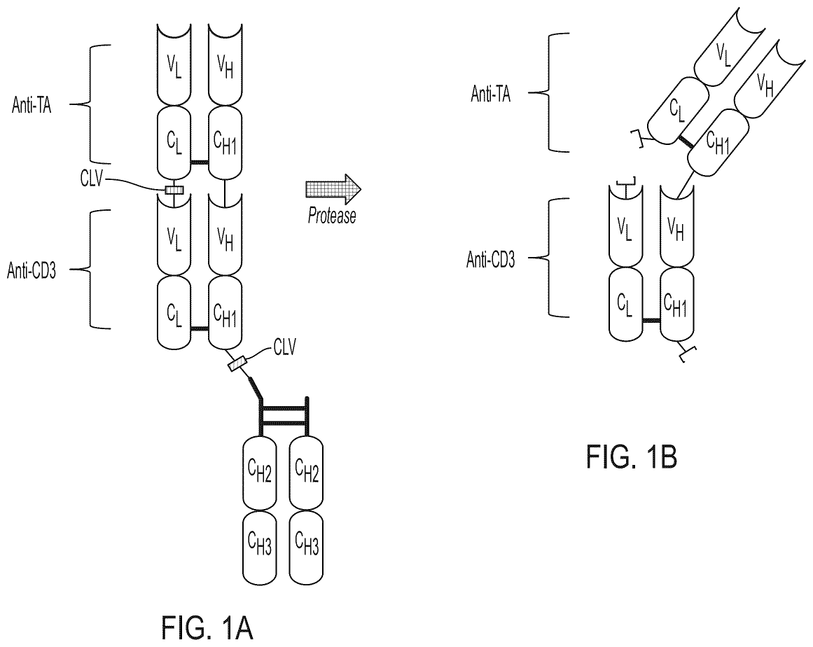

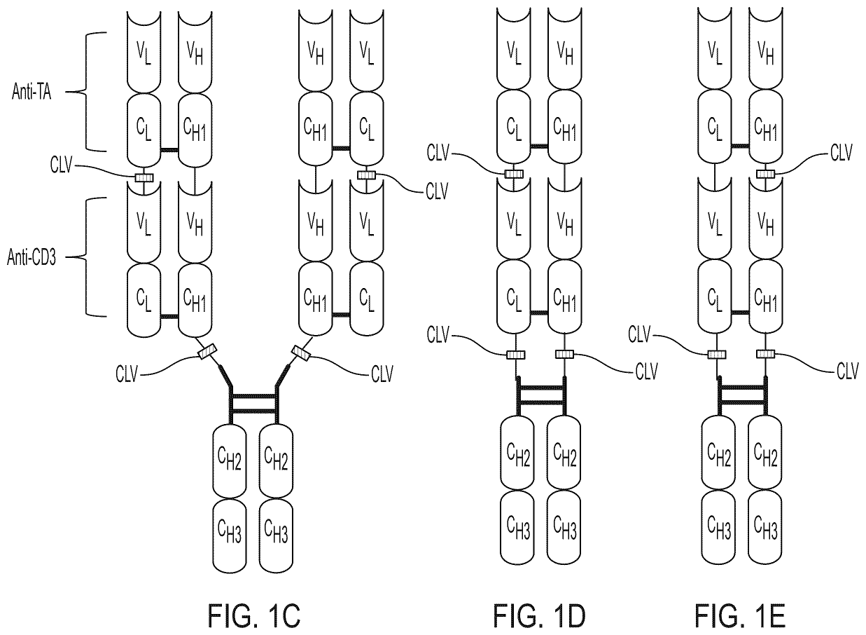

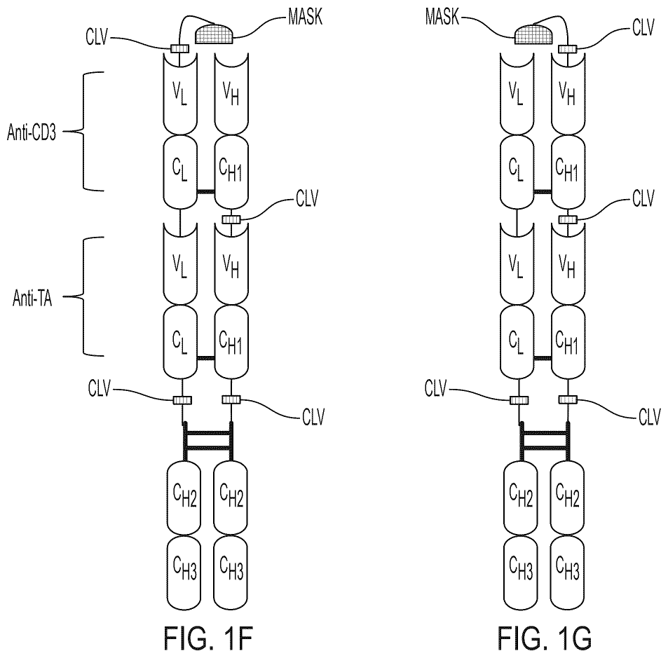

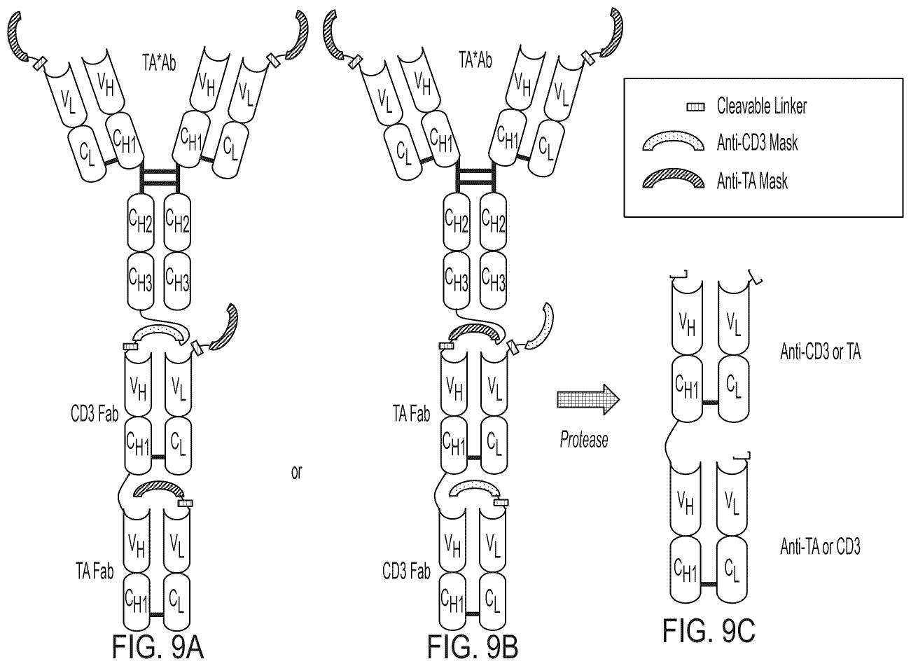

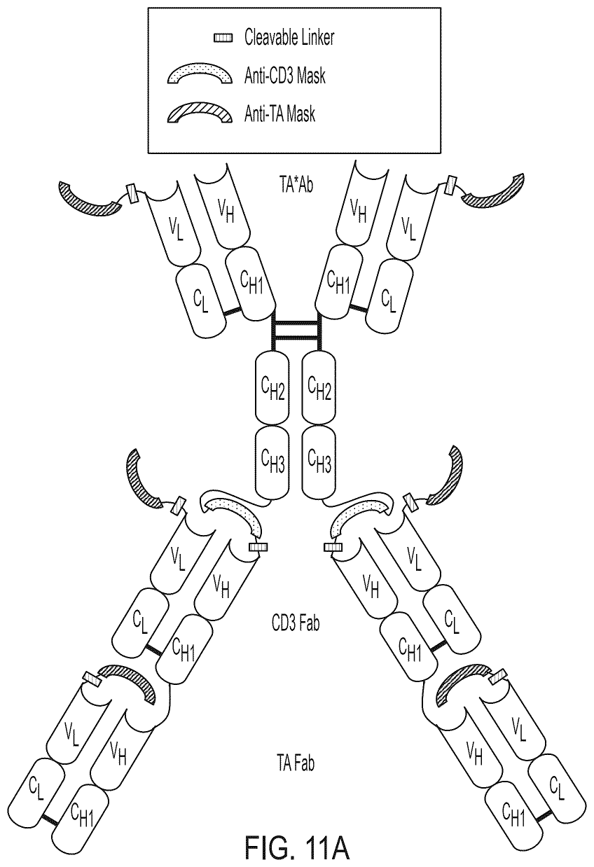

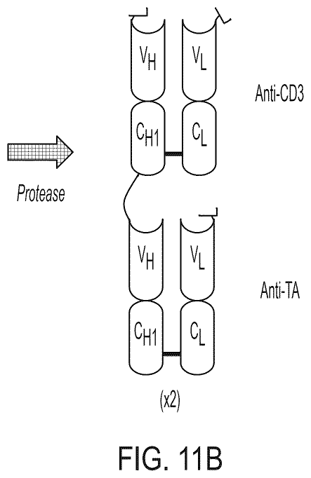

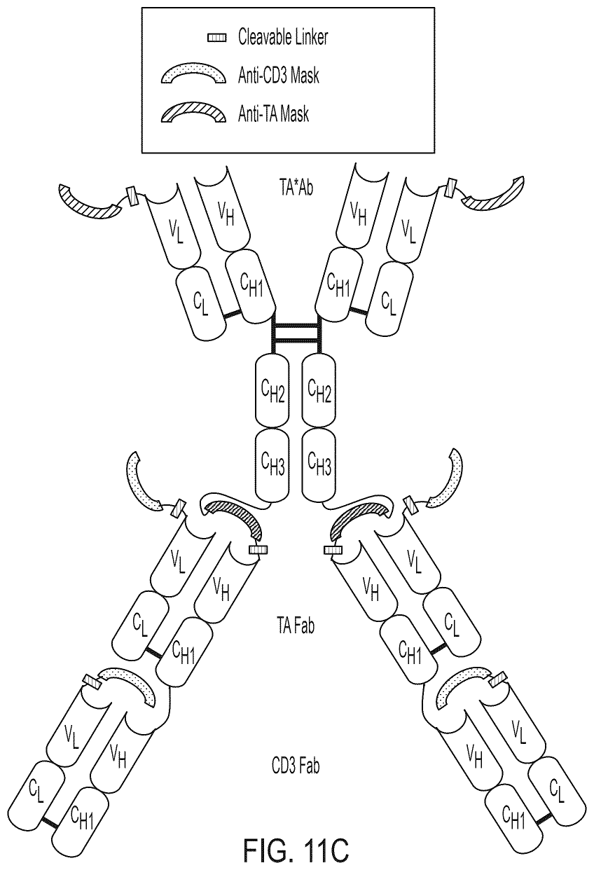

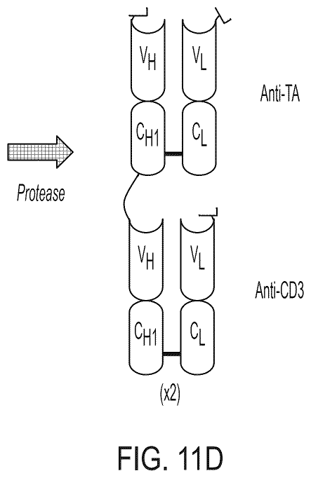

In various embodiments, the multispecific antigen-binding molecules of the present disclosure comprise the proprotein structures of the molecules illustrated in FIG. 1A, 1C, 1D, 2A, 3A, 4A, 4B, 4C, 4D, 4E, 5A, 5C, 6A, 7A, 8A, 8B, 8D, 8E, 8G, 8H, 8J, 8K, 9A, 9B, 10A, 10C, 11A or 11C. The present disclosure also includes activated molecules comprising the structures illustrated in FIG. 1B, 2B, 3B, 5B, 5D, 6B, 7B, 8C, 8F, 81, 8L, 9C, 10B, 10D, 11B or 11D. As illustrated, the molecules may include a disulfide bond between the constant regions of one immunoglobulin Fab, or both immunoglobulin Fabs, or all immunoglobulin Fabs, and the disulfide bond, if present, may be connecting the constant domains or the variable domains of an immunoglobulin Fab. The proprotein structures including an immunoglobulin Fc region can extend the half-life of the molecules until they reach the tumor microenvironment where proteolytic cleavage activates the molecules by making the antigen-binding regions of the tandem Fabs accessible for binding to the respective target or T-cell antigens. Elimination of the immunoglobulin Fc region in these instances also reduces the half-life of the unmasked and active molecules to improve safety while providing a therapeutic effect. In embodiments comprising an antibody encompassing the immunoglobulin Fc region (e.g., the molecules of FIGS. 4F, 4G, 9A, 9B, 11A and 11C), the antibody (e.g., anti-TA* antibody) may be used to target a tumor-associated antigen to deliver the masked payload to the tumor environment where proteolytic cleavage will produce the activated molecule and a therapeutic effect. The antigen targeted by the anti-TA* antibody may be the same or different from the antigen bound by the TA Fabs of the molecules discussed herein.

In some embodiments, one or more Fabs (e.g., the second immunoglobulin Fab, or the second immunoglobulin Fab and the fourth immunoglobulin Fab) include cysteine mutations at residue 44 of the HCVR and residue 100 of the LCVR (Kabat numbering) to produce inter-disulfide bonding between the variable regions.

In various embodiments, the LCVR (and optionally the CL) of any of the antigen-binding domains can be a cognate LCVR that corresponds to the HCVR, or the LCVR can be a universal LCVR (and optionally CL) common to multiple antigen-binding domains. In some embodiments, the light chain of the Fab domains is a common light chain (e.g., a universal light chain). In some embodiments, the light chain of the Fab domains is a cognate light chain corresponding to the target antigen binding domain, and the light chain is common to both Fab domains.

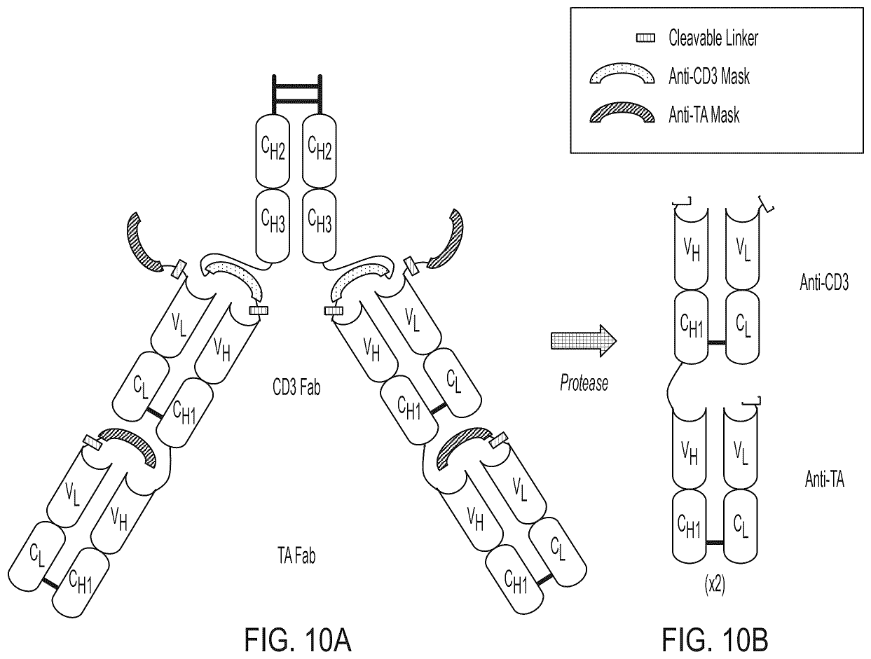

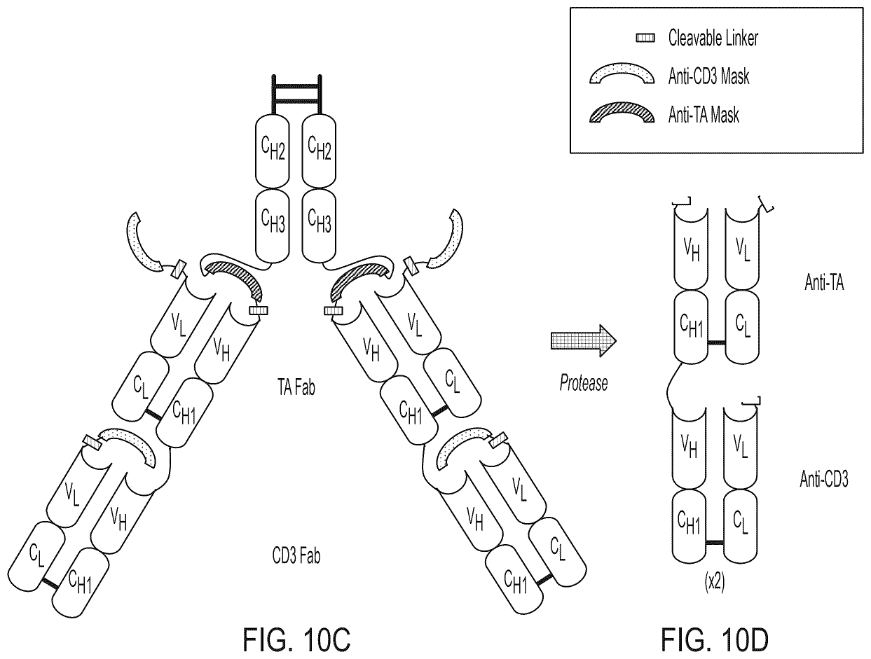

In some cases, the multispecific molecule (e.g., of FIG. 1C or 4C or 10A or 10C, or 11A or 11C) binds distinct target antigens (different epitopes on the same protein, or different proteins). In some cases, the distinct target antigens (first and second target antigens) are expressed on the surface of the same target cell (e.g., tumor cell). In some cases, the multispecific molecule (e.g., of FIG. 1C or 4C or 10A or 10C, or 11A or 11C) binds the same target antigen (the same epitope on the same protein). In various embodiments, the T cell antigen-binding domains of the multispecific molecules (e.g., FIG. 1C or 4C or 10A or 10C, or 11A or 11C) bind the same or distinct (different epitopes on the same protein, or different proteins) T-cell antigens. In some cases, the distinct T-cell antigens are a co-stimulatory molecule (e.g., CD28) and a check-point inhibitor (e.g., PD-1) on the surface of a T cell. In such embodiments, the multispecific molecules of the disclosure can provide a costimulatory signal to the T cell as well as prevent checkpoint inhibition. As used herein, reference to the “same” target antigen or T-cell antigen does not necessarily mean that the antigen-binding domains are binding to the same surface molecule, but rather that the antigen-binding domains have the same specificity (e.g., they each bind CD3 or a TA). Similarly, references to a “distinct” target antigen or T-cell antigen mean that it is different from another target antigen (e.g., PSMA vs. MUC16) or another T-cell antigen (e.g., CD28 vs. PD-1), or is another epitope on the same protein.

In any of the embodiments discussed above or herein, the target antigen can be a tumor-associated antigen.

In some cases, the target antigen is a peptide in the context of the groove (PiG) of a major histocompatibility complex (MHC) protein. In some embodiments, the PiG is a peptide consisting of about 5 to about 40 amino acid residues, from about 6 to about 30 amino acid residues, from about 8 to about 20 amino acid residues, or about 9, 10, or 11 amino acid residues. In some cases, the PiG is a fragment of a tumor-associated antigen. In various embodiments, the target antigen is a peptide in the context of the groove of any class, subtype or allele of human leukocyte antigen, including any of HLA-A, HLA-B, HLA-C, HLA-DR, HLA-DQ or HLA-DP. In some embodiments, the target antigen is a peptide/MHC complex. In some cases, the peptide in the peptide/MHC complex is a fragment of a tumor-associated antigen.

In some cases, the antigen is a tumor-associated antigen or an antigen expressed by a tumor cell. In some embodiments, the tumor-associated antigen such as AFP, ALK, BAGE proteins, BCMA, BIRC5 (survivin), BIRC7, β-catenin, brc-abl, BRCA1, BORIS, CA9, carbonic anhydrase IX, caspase-8, CALR, CCR5, CD19, CD20 (MS4A1), CD22, CD40, CD70, CDK4, CEA, CLDN18.2, cyclin-B1, CYP1B1, DLL3, ErbB1/Her1, EGFRvIII, ErbB2/Her2, ErbB3, ErbB4, ETV6-AML, EpCAM, EphA2, Fra-1, FOLR1, GAGE proteins (e.g., GAGE-1, -2), GD2, GD3, GloboH, glypican-3, GM3, gp100, Her2, HLA/B-raf, HLA/k-ras, HLA/MAGE-A3, hTERT, IL-10, LMP2, MAGE proteins (e.g., MAGE-1, -2, -3, -4, -6, and -12, MAGE-A4), MART-1, mesothelin, ML-IAP, Muc1, Muc2, Muc3, Muc4, Muc5, Muc16 (CA-125), MUM1, NA17, NY-BR1, NY-BR62, NY-BR85, NY-ESO1, p15, p53, PAP, PAX3, PAX5, PCTA-1, PLAC1, PRLR, PRAME, PSMA (FOLH1), RAGE proteins, Ras, RGS5, Rho, SART-1, SART-3, STEAP1, STEAP2, TAG-72, TGF-β, TMPRSS2, Thompson-nouvelle antigen (Tn), TRP-1, TRP-2, tyrosinase, or uroplakin-3.

In any of the embodiments discussed above or herein, the T cell antigen can be an antigen expressed at the surface of a T cell, a T cell receptor complex antigen, a co-stimulatory molecule or a check point inhibitor on a T cell, CD2, CD3, CD27, CD28, 4-1BB or PD-1. In some cases, the T cell antigen is a T cell receptor complex antigen. In some cases, the T cell antigen is CD3. In some cases, the T cell antigen is a co-stimulatory molecule or a check-point inhibitor on a T cell. In some cases, the T cell antigen is selected from the group consisting of CD27, CD28, 4-1BB and PD-1. In some cases, the T cell antigen is selected from the group consisting of CD3, CD27, CD28, 4-1BB and PD-1. In some cases, the T cell antigen is selected from the group consisting of CD28, ICOS, HVEM, CD27, 4-1BB, 0X40, DR3, GITR, CD30, SLAM, CD2, 2B4, CD226, TIM1, and TIM2.

In certain embodiments in which the T cell antigen is CD3, the CD3-binding domain binds to human CD3 and induces human T cell activation. In certain embodiments, the CD3-binding domain binds weakly to human CD3 and induces human T cell activation. In some embodiments, the CD3-binding domain binds weakly to human CD3 and induces tumor-associated antigen-expressing cell killing. In some embodiments, the CD3-binding domain binds or associates weakly with human and cynomolgus (monkey) CD3, yet the binding interaction is not detectable by in vitro assays known in the art. In some embodiments, the CD3-binding domain binds with weak affinity to human CD3. In some embodiments, the CD3-binding domain binds with moderate affinity to human CD3. In some embodiments, the CD3-binding domain binds with high affinity to human CD3. In some embodiments, the CD3-binding domain binds to human CD3 (e.g., at 25° C. or 37° C.) with a K_{D} of less than about 15 nM as measured by surface plasmon resonance (e.g., mAb-capture or antigen-capture format) or a substantially similar assay. In some embodiments, the CD3-binding domain binds human CD3 with a K_{D} value of greater than about 15 nM, greater than about 20 nM, greater than about 30 nM, greater than about 40 nM, greater than about 50 nM, greater than about 60 nM, greater than about 100 nM, greater than about 200 nM, or greater than about 300 nM, as measured in a surface plasmon resonance binding assay (e.g., mAb-capture or antigen-capture format) or a substantially similar assay. In some embodiments, the antibodies or antigen-binding fragments of the present disclosure bind CD3 with a K_{D} of less than about 5 nM, less than about 2 nM, less than about 1 nM, less than about 800 pM, less than about 600 pM, less than about 500 pM, less than about 400 pM, less than about 300 pM, less than about 200 pM, less than about 180 pM, less than about 160 pM, less than about 140 pM, less than about 120 pM, less than about 100 pM, less than about 80 pM, less than about 60 pM, less than about 40 pM, less than about 20 pM, or less than about 10 pM, as measured by surface plasmon resonance, e.g., using a mAb-capture or antigen-capture assay, or a substantially similar assay. In some embodiments, the CD3-binding domain binds human CD3 with a K_{D} of 1 nM to 19 nM, as measured by surface plasmon resonance, e.g., using a mAb-capture or antigen-capture assay, or a substantially similar assay. In some embodiments, the CD3-binding domain binds human CD3 with a K_{D} of 20 nM to 99 nM, as measured by surface plasmon resonance, e.g., using a mAb-capture or antigen-capture assay, or a substantially similar assay. In some embodiments, the CD3-binding domain binds human CD3 with a K_{D} of 100 nM to 500 nM, as measured by surface plasmon resonance, e.g., using a mAb-capture or antigen-capture assay, or a substantially similar assay. In some embodiments, the CD3-binding domain binds human CD3 with a K_{D} of 500 nM to 1 μM, as measured by surface plasmon resonance, e.g., using a mAb-capture or antigen-capture assay, or a substantially similar assay. In some embodiments, the CD3-binding domain binds human CD3 with a K_{D} of 500 nM to 10 μM, as measured by surface plasmon resonance, e.g., using a mAb-capture or antigen-capture assay, or a substantially similar assay. In some embodiments, the CD3-binding domain binds human CD3 with a K_{D} of 500 nM to 100 μM, as measured by surface plasmon resonance, e.g., using a mAb-capture or antigen-capture assay, or a substantially similar assay. In any of these assays, the K_{D} may be measured at, e.g., 25° C. or 37° C.

In some embodiments, the CD3-binding domain exhibits an EC₅₀ value of less than less than about 50 nM, less than about 40 nM, less than about 30 nM, less than about 20 nM, less than about 10 nM, less than about 5 nM, less than about 4 nM, less than about 3 nM, less than about 2 nM, less than about 1 nM, less than 900 pM, less than 800 pM, less than 700 pM, less than 600 pM, or less than 500 pM, as measured in an in vitro flow cytometry binding assay. In some embodiments, the CD3-binding domain exhibits an EC₅₀ value of about or greater than about 1 nM, 2 nM, 3 nM, 4 nM, 5 nM, 10 nM, 25 nM, 50 nM, 100 nM, 500 nM or 1 μM, as measured in an in vitro flow cytometry binding assay.

In any of the embodiments, the CD3-binding domain can comprise any of the HCVR/LCVR or CDR (e.g., the six CDRs contained within a pair of HCVR/LCVR sequences) amino acid sequences of the anti-CD3 antibodies disclosed in WO 2014/047231 (9250-WO) or WO 2017/053856 (10151WO01), including the antibodies identified as 7195P, 7221G, 7221G5 and 7221G20. In various embodiments, an anti-CD3 antibody identified as a “strong binder” has an affinity for human CD3 in the single digit nanomolar range (e.g., from 1-9 nM) or lower (e.g., picomolar range, such as 30-40 pM) as measured in a surface plasmon resonance assay (e.g., at 25° C. in an antigen-capture format with measurements conducted on a T200 BIACORE instrument). In various embodiments, an anti-CD3 antibody identified as a “moderate binder” has an affinity for human CD3 in the double digit nanomolar range (e.g., from 10-99 nM, optionally from 10-50 nM or 10-25 nM) as measured in a surface plasmon resonance assay. In various embodiments, an anti-CD3 antibody identified as a “weak binder” has an affinity for human CD3 in the three digit nanomolar range (e.g., from 100-999 nM, optionally from 100-500 nM or from 500 nM to 1 μM) as measured in a surface plasmon resonance assay. In various embodiments, an anti-CD3 antibody identified as a “very weak binder” has an affinity for human CD3 that is greater than 10 μM or is undetectable as measured in a surface plasmon resonance assay. In some embodiments, it is advantageous to include a human CD3 binding domain that binds with a “weak” or “very weak” affinity for human CD3, e.g., an affinity of 250 nM to 500 nM, or 500 nM to 10 μM, or 500 nM to 100 μM. This “weak” or “very weak” binding affinity may increase the difference in cytotoxic potency between masked and non-masked constructs (i.e., the construct prior to and after, respectively, contact with a protease capable of cleaving the protease-cleavable linkers within the construct). This enhances the safety of the constructs prior to processing by a protease, while maintaining cytotoxic potency of the cleaved construct. In some embodiments, CD3 binding domains comprising the HCVR sequences (CDRs and/or variable domains) of the anti-CD3 antibodies designated “G5” or “G20” in Table 1, coupled with the LCVR sequences (CDRs and/or variable domain) in Table 2 are advantageous, and provide “weak” or “very weak” binding to human CD3 while maintaining the ability to induce T-cell activation and produce therapeutic cytotoxic effects in tumor cells.

In any of the embodiments, the CD3-binding domain can comprise any of the HCVR/LCVR or CDR (e.g., the six CDRs contained within a pair of HCVR/LCVR sequences) amino acid sequences set forth in the following tables (the “G” versions are taken from WO 2017/053856). In some embodiments, the CD3-binding domains comprise a cognate light chain corresponding to the target antigen binding domain. In other words, the cognate light chain of the target antigen binding domain is common to both the target antigen-binding domain and the CD3-binding domain. In some embodiments, the CD3-binding domains comprise a universal light chain, such as that set forth in SEQ ID NO: 162.

TABLE 1
Heavy Chain Amino Acid Sequence Identifiers

| Antibody | |
|---|---|
| CD3-VH | SEQ ID NOs: |

| Designation | HCVR | CDR1 | CDR2 | CDR3 |
|---|---|---|---|---|
| CD3-VH-G | 2 | 4 | 6 | 8 |
| CD3-VH-G2 | 10 | 12 | 14 | 16 |
| CD3-VH-G3 | 18 | 20 | 22 | 24 |
| CD3-VH-G4 | 26 | 28 | 30 | 32 |
| CD3-VH-G5 | 34 | 36 | 38 | 40 |
| CD3-VH-G8 | 42 | 44 | 46 | 48 |
| CD3-VH-G9 | 50 | 52 | 54 | 56 |
| CD3-VH-G10 | 58 | 60 | 62 | 64 |
| CD3-VH-G11 | 66 | 68 | 70 | 72 |
| CD3-VH-G12 | 74 | 76 | 78 | 80 |
| CD3-VH-G13 | 82 | 84 | 86 | 88 |
| CD3-VH-G14 | 90 | 92 | 94 | 96 |
| CD3-VH-G15 | 98 | 100 | 102 | 104 |
| CD3-VH-G16 | 106 | 108 | 110 | 112 |
| CD3-VH-G17 | 114 | 116 | 118 | 120 |
| CD3-VH-G18 | 122 | 124 | 126 | 128 |
| CD3-VH-G19 | 130 | 132 | 134 | 136 |
| CD3-VH-G20 | 138 | 140 | 142 | 144 |
| CD3-VH-G21 | 146 | 148 | 150 | 152 |
| 7195P | 154 | 156 | 158 | 160 |

TABLE 2
Heavy Chain Nucleic Acid Sequence Identifiers

| Antibody | |
|---|---|
| CD3-VH | SEQ ID NOs: |

| Designation | HCVR | CDR1 | CDR2 | CDR3 |
|---|---|---|---|---|
| CD3-VH-G | 1 | 3 | 5 | 7 |
| CD3-VH-G2 | 9 | 11 | 13 | 15 |
| CD3-VH-G3 | 17 | 19 | 21 | 23 |
| CD3-VH-G4 | 25 | 27 | 29 | 31 |
| CD3-VH-G5 | 33 | 35 | 37 | 39 |
| CD3-VH-G8 | 41 | 43 | 45 | 47 |
| CD3-VH-G9 | 49 | 51 | 53 | 55 |
| CD3-VH-G10 | 57 | 59 | 61 | 63 |
| CD3-VH-G11 | 65 | 67 | 69 | 71 |
| CD3-VH-G12 | 73 | 75 | 77 | 79 |
| CD3-VH-G13 | 81 | 83 | 85 | 87 |
| CD3-VH-G14 | 89 | 91 | 93 | 95 |
| CD3-VH-G15 | 97 | 99 | 101 | 103 |
| CD3-VH-G16 | 105 | 107 | 109 | 111 |
| CD3-VH-G17 | 113 | 115 | 117 | 119 |
| CD3-VH-G18 | 121 | 123 | 125 | 127 |
| CD3-VH-G19 | 129 | 131 | 133 | 135 |
| CD3-VH-G20 | 137 | 139 | 141 | 143 |
| CD3-VH-G21 | 145 | 147 | 149 | 151 |
| 7195P | 153 | 155 | 157 | 159 |

TABLE 3
Light Chain Amino Acid Sequence Identifiers

| | |
|---|---|
| Antibody | |
| ULC | SEQ ID NOs: |

| Designation | LCVR | CDR1 | CDR2 | CDR3 |
|---|---|---|---|---|
| Vκ1-39JK5 | 162 | 164 | 166 | 168 |
| | | | (AAS) | |

TABLE 4
Light Chain Nucleic Acid Sequence Identifiers

| Antibody | | | | |
|---|---|---|---|---|
| ULC | SEQ ID NOS: | | | |
| Designation | LCVR | CDR1 | CDR2 | CDR3 |
| Vκ1-39JK5 | 161 | 163 | 165 | 167 |
| | | | (gctgcatcc) | |

Each of the antibodies set forth in Table 1 comprises a common light chain variable region comprising the amino acid sequence set forth in Table 3. Each of the “G” designated antibodies may also be referred to herein with a “7221” prefix, e.g., 7221G, 7221G5, 7221G20, etc.

The multispecific antigen-binding molecules of the disclosure can comprise an Fc domain comprising a hinge domain at its N-terminus. The hinge region can be a native or a modified hinge region. Hinge regions are typically found at the N-termini of Fc regions. The term “hinge domain”, unless the context dictates otherwise, refers to a naturally or non-naturally occurring hinge sequence that in the context of a single or monomeric polypeptide chain is a monomeric hinge domain and in the context of a dimeric polypeptide can comprise two associated hinge sequences on separate polypeptide chains. Sometimes, the two associated hinge sequences are referred to as a “hinge region”. In any of the embodiments comprising an immunoglobulin Fc region, the hinge region may be present or the hinge region may be absent. In the case in which the hinge is absent, only pairs of polypeptides comprising a CH2 domain and a CH3 domain will be present, and the two polypeptides can be linked by a non-cleavable linker or by an interchain disulfide bond.

In various embodiments in which the multispecific antigen-binding molecules include a hinge domain or a hinge region, the constant region may be chimeric, combining sequences derived from more than one immunoglobulin isotype. For example, a chimeric Fc domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge domain. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antigen-binding molecules set forth herein comprises, from N- to C-terminus: [IgG4 C_{H}1]-[IgG4 upper hinge]-[IgG2 lower hinge]-[IgG4 CH2]-[IgG4 CH3]. Another example of a chimeric Fc domain that can be included in any of the antigen-binding molecules set forth herein comprises, from N- to C-terminus: [IgG1 C_{H}1]-[IgG1 upper hinge]-[IgG2 lower hinge]-[IgG4 CH2]-[IgG1 CH3]. These and other examples of chimeric Fc domains that can be included in any of the antigen-binding molecules of the present disclosure are described in WO 2014/121087 (8550-WO). Chimeric Fc domains having these general structural arrangements, and variants thereof, can have altered Fc receptor binding, which in turn affects Fc effector function.

In various embodiments in which the multispecific antigen-binding molecules include a hinge domain or a hinge region, positions 233-236 within the hinge domain may be G, G, G and unoccupied; G, G, unoccupied, and unoccupied; G, unoccupied, unoccupied, and unoccupied; or all unoccupied, with positions numbered by EU numbering. Optionally, the heavy chain constant region comprises from N-terminal to C-terminal the hinge domain, a CH2 domain and a CH3 domain. Optionally, the hinge region, if any, CH2 region and CH3 region are the same human isotype. Optionally, the hinge region, if any, CH2 region and CH3 region are human IgG1. Optionally, the hinge region, if any, CH2 region and CH3 region are human IgG2. Optionally, the hinge region, if any, CH2 region and CH3 region are human IgG4. Optionally, the constant region has a CH3 domain modified to reduce binding to protein A. These and other examples of multimerizing heavy chain constant regions that can be included in any of the antigen-binding molecules of the present disclosure are described in WO 2016/161010 (10140WO01).

In some embodiments, the first and second Fc domains may be of the same IgG isotype such as, e.g., IgG1/IgG1, IgG2/IgG2, IgG4/IgG4. Alternatively, the first and second Fc domains may be of different IgG isotypes such as, e.g., IgG1/IgG2, IgG1/IgG4, IgG2/IgG4, etc.

In certain embodiments, the Fc region may be replaced with a multimerizing domain including peptides or polypeptides comprising or consisting of a leucine zipper, a helix-loop motif, or a coiled-coil motif, or other such structures known in the art.

The multimerizing domains, e.g., Fc domains (with or without a hinge), may comprise one or more amino acid changes (e.g., insertions, deletions or substitutions) as compared to the wild-type, naturally occurring version of the Fc domain. For example, the disclosure includes bispecific antigen-binding molecules comprising one or more modifications in the Fc domain that results in a modified Fc domain having a modified binding interaction (e.g., enhanced or diminished) between Fc and FcRn. In one embodiment, the bispecific antigen-binding molecule comprises a modification in a C_{H}2 or a C_{H}3 region, wherein the modification increases the affinity of the Fc domain to FcRn in an acidic environment (e.g., in an endosome where pH ranges from about 5.5 to about 6.0). Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P).

The present disclosure also includes multispecific antigen-binding molecules comprising a first Ig C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). See, for example, U.S. Pat. No. 8,586,713. Further modifications that may be found within the second C_{H}3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies.

### Preparation of Antigen-Binding Domains and Construction of Multispecific Molecules

Antigen-binding domains specific for particular antigens can be prepared by any antibody generating technology known in the art. Once obtained, different antigen-binding domains, specific for two or more different antigens (e.g., CD3 and a target antigen), can be appropriately arranged relative to one another to produce the structures of the multispecific antigen-binding molecules of the present disclosure using routine methods. In certain embodiments, one or more of the individual components (e.g., heavy and light chains or parts thereof) of the multispecific antigen-binding molecules of the disclosure are derived from chimeric, humanized or fully human antibodies. Methods for making such antibodies are well known in the art. For example, one or more of the heavy and/or light chains of the multispecific antigen-binding molecules of the present disclosure can be prepared using VELOCIMMUNE™ technology. Using VELOCIMMUNE™ technology (or any other human antibody generating technology), high affinity chimeric antibodies to a particular antigen (e.g., CD3 or a target antigen) are initially isolated having a human variable region and a mouse constant region. The antibodies are characterized and selected for desirable characteristics, including affinity, selectivity, epitope, etc. The mouse constant regions are replaced with a desired human constant region to generate fully human heavy and/or light chains that can be incorporated into the multispecific antigen-binding molecules of the present disclosure.

Genetically engineered animals may be used to make human multispecific antigen-binding molecules. For example, a genetically modified mouse can be used which is incapable of rearranging and expressing an endogenous mouse immunoglobulin light chain variable sequence, wherein the mouse expresses only one or two human light chain variable domains encoded by human immunoglobulin sequences operably linked to the mouse kappa constant gene at the endogenous mouse kappa locus. Such genetically modified mice can be used to produce fully human multispecific antigen-binding molecules comprising two different heavy chains that associate with an identical light chain that comprises a variable domain derived from one of two different human light chain variable region gene segments. (See, e.g., US 2011/0195454). Fully human refers to an antibody, or antigen-binding fragment or immunoglobulin domain thereof, comprising an amino acid sequence encoded by a DNA derived from a human sequence over the entire length of each polypeptide of the antibody or antigen-binding fragment or immunoglobulin domain thereof. In some instances, the fully human sequence is derived from a protein endogenous to a human. In other instances, the fully human protein or protein sequence comprises a chimeric sequence wherein each component sequence is derived from human sequence. While not being bound by any one theory, chimeric proteins or chimeric sequences are generally designed to minimize the creation of immunogenic epitopes in the junctions of component sequences, e.g. compared to any wild-type human immunoglobulin regions or domains.

In various embodiments, the methods and techniques discussed above are used to generate antibodies to a T-cell antigen and a target antigen, and the antigen-binding domains of these antibodies (e.g., the HCVR, LCVR, or CDRs) are used to produce the multispecific antigen-binding molecules as discussed herein or having, e.g., the structures illustrated in the figures.

### Binding Properties of the Antigen-Binding Domains

As used herein, the term “binding” in the context of the binding of an antibody (e.g., a corresponding antibody), immunoglobulin, antigen-binding domain or multispecific antigen-binding molecule to, e.g., a predetermined antigen, such as a cell surface protein or fragment thereof, typically refers to an interaction or association between a minimum of two entities or molecular structures, such as an antigen-binding domain/antigen interaction.

For instance, binding affinity typically corresponds to a K_{D} value of about 10⁻⁷ M or less, such as about 10⁻⁸ M or less, such as about 10⁻⁹ M or less when determined by, for instance, surface plasmon resonance (SPR) technology in a BIAcore 3000 instrument using the antigen as the ligand and the antibody, Ig, antibody-binding domain or multispecific antigen-binding molecule as the analyte (or anti-ligand). Flow cytometry assays are also routinely used.

Accordingly, the antibody (e.g., a corresponding antibody), antigen-binding domain or multispecific antigen-binding molecule of the disclosure binds to the predetermined antigen or cell surface molecule having an affinity corresponding to a K_{D} value that is at least ten-fold lower than its affinity for binding to a non-specific antigen (e.g., BSA, casein). According to the present disclosure, the affinity of an antibody (e.g., a corresponding antibody), antigen-binding domain or multispecific antigen-binding molecule corresponding to a K_{D} value that is equal to or less than ten-fold lower than a non-specific antigen may be considered non-detectable binding, however such an antibody may be paired with a second antigen binding arm for the production of a multispecific molecule of the disclosure.

The term “K_{D}” (M) refers to the dissociation equilibrium constant of a particular antibody (or antigen-binding domain)-antigen interaction, or the dissociation equilibrium constant of an antibody (or antigen-binding domain) or antibody-binding fragment binding to an antigen. There is an inverse relationship between K_{D} and binding affinity, therefore the smaller the K_{D} value, the higher, i.e. stronger, the affinity. Thus, the terms “higher affinity” or “stronger affinity” relate to a higher ability to form an interaction and therefore a smaller K_{D} value, and conversely the terms “lower affinity” or “weaker affinity” relate to a lower ability to form an interaction and therefore a larger K_{D} value. In some circumstances, a higher binding affinity (or K_{D}) of a particular molecule (e.g. antibody or antigen-binding domain) to its interactive partner molecule (e.g. antigen X) compared to the binding affinity of the molecule (e.g. antibody or antigen-binding domain) to another interactive partner molecule (e.g. antigen Y) may be expressed as a binding ratio determined by dividing the larger K_{D} value (lower, or weaker, affinity) by the smaller K_{D} (higher, or stronger, affinity), for example expressed as 5-fold or 10-fold greater binding affinity, as the case may be.

The term “k_{d}” (sec-1 or 1/s) refers to the dissociation rate constant of a particular antibody (or antigen-binding domain)-antigen interaction, or the dissociation rate constant of an antibody or antibody-binding domain. Said value is also referred to as the k_{off} value.

The term “kₐ” (M-1×sec-1 or 1/M) refers to the association rate constant of a particular antibody (or antigen-binding domain)-antigen interaction, or the association rate constant of an antibody or antibody-binding domain.

The term “K_{A}” (M-1 or 1/M) refers to the association equilibrium constant of a particular antibody (or antigen-binding domain)-antigen interaction, or the association equilibrium constant of an antibody or antibody-binding domain. The association equilibrium constant is obtained by dividing the kₐ by the k_{d}.

The term “EC50” or “EC₅₀” refers to the half maximal effective concentration, which includes the concentration of an antibody (or antigen-binding domain or multispecific molecule) which induces a response halfway between the baseline and maximum after a specified exposure time. The EC₅₀ essentially represents the concentration of an antibody (or antigen-binding domain or multispecific molecule) where 50% of its maximal effect is observed. In certain embodiments, the EC₅₀ value equals the concentration of a multispecific molecule of the disclosure that gives half-maximal binding to cells expressing CD3 or target antigen (e.g., tumor-associated antigen), as determined by e.g. a flow cytometry binding assay. Thus, reduced or weaker binding is observed with an increased EC₅₀, or half maximal effective concentration value.

In one embodiment, decreased binding can be defined as an increased EC₅₀ molecule concentration which enables binding to the half-maximal amount of target cells.

In another embodiment, the EC₅₀ value represents the concentration of a molecule of the disclosure that elicits half-maximal depletion of target cells by T cell cytotoxic activity. Thus, increased cytotoxic activity (e.g. T cell-mediated tumor cell killing) is observed with a decreased EC₅₀, or half maximal effective concentration value.

The present disclosure includes antigen-binding domains and multispecific antigen-binding molecules with pH-dependent binding characteristics. For example, a molecule of the present disclosure may exhibit reduced binding to a T-cell antigen or a target antigen at acidic pH as compared to neutral pH. Alternatively, molecules of the disclosure may exhibit enhanced binding to a T-cell antigen or a target antigen at acidic pH as compared to neutral pH. The expression “acidic pH” includes pH values less than about 6.2, e.g., about 6.0, 5.95, 5,9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding . . . at acidic pH as compared to neutral pH” is expressed in terms of a ratio of the K_{D} value of the molecule (or antigen-binding domain) binding to its antigen at acidic pH to the K_{D} value of the molecule (or antigen-binding domain) binding to its antigen at neutral pH (or vice versa). For example, a molecule or antigen-binding domain may be regarded as exhibiting “reduced binding to a T-cell antigen or a target antigen at acidic pH as compared to neutral pH” for purposes of the present disclosure if the molecule or antigen-binding domain exhibits an acidic/neutral K_{D} ratio of about 3.0 or greater. In certain exemplary embodiments, the acidic/neutral K_{D} ratio for a molecule or antigen-binding domain of the present disclosure can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0. 25.0, 30.0, 40.0, 50.0, 60.0, 70.0, 100.0 or greater.

Multispecific molecules with pH-dependent binding characteristics may be obtained, e.g., by screening a population of corresponding antibodies for reduced (or enhanced) binding to a particular antigen at acidic pH as compared to neutral pH. Additionally, modifications of the antigen-binding domain at the amino acid level may yield molecules with pH-dependent characteristics. For example, by substituting one or more amino acids of an antigen-binding domain (e.g., within a CDR) with a histidine residue, a molecule with reduced antigen-binding at acidic pH relative to neutral pH may be obtained.

### Biological Characteristics of the Multispecific Antigen-Binding Molecules

The present disclosure includes multispecific antigen-binding molecules and antigen-binding domains thereof that are capable of simultaneously binding to a human T-cell antigen (e.g., CD3) and a human target antigen or antigens (e.g., a tumor-associated antigen).

The present disclosure includes multispecific antigen-binding molecules that bind a human T-cell antigen (e.g., CD3) and induce T cell activation in the presence of target cells. For example, in some embodiments, the present disclosure includes multispecific antigen-binding molecules that bind a human T-cell antigen (e.g., CD3) and induce T cell cytotoxic activity in the presence of cells expressing the target antigen or target antigens (e.g., a tumor-associated antigen).

The present disclosure includes multispecific antigen-binding molecules that are capable of depleting or reducing cell populations in which the cells express the target antigen or target antigens. The multispecific antigen-binding molecules of the present disclosure are capable of inducing T-cell mediated cytotoxicity.

The present disclosure includes multispecific antigen-binding molecules that bind a human T-cell antigen (e.g., CD3) and two distinct target antigens (e.g., a molecule having the structure of FIG. 1C), and induce cytotoxic activity and/or T-cell activation in the presence of cells expressing the two target antigens.

Many cancers express a variety of intracellular antigens that are processed inside the cell by the proteosome and associated peptides are presented at the surface of the cell in the context of HLA molecules. Targeting peptides from different proteins may be used to increase the specificity of the multispecific molecules of the present disclosure. In some cases, cancers characterized by PiG antigens or low density cancer antigens escape conventional cancer therapies because they are often present in low target copy numbers within tumors. Additionally, solid tumors characterized by PiGs or low density cancer antigens can be more resistant to therapy and more difficult to treat because they are not cell surface antigens, but are present in grooves within the cancer related peptide. Thus, use of a multispecific molecule of the present disclosure targeting two distinct antigens (e.g., low density antigens) can effectively target PiGs and/or low density cancer antigens to increase/enhance efficacy of therapy in cancers, especially those cancers characterized by solid tumors.

In various embodiments, the multispecific antigen-binding molecules of the present disclosure are capable of inducing T-cell mediated cytotoxicity in cell populations when the density of the target antigen ranges from about 10 copies per cell to about 1 million copies per cell or more. In some cases, the target antigen is present at a copy number/cell of about 10, about 20, about 30, about 40, about 50, about 60, about 70, about 80, about 90, about 100, about 150, about 200, about 250, about 300, about 350, about 400, about 450, about 500, about 600, about 700, about 800, about 900, about 1000, about 1500, about 2000, about 2500, about 3000, about 3500, about 4000, about 4500, about 5000, about 6000, about 7000, about 8000, about 9000, about 10000, about 15000, about 20000, about 25000, about 30000, about 35000, about 40000, about 45000, about 50000, about 75000, about 100000 (i.e., 100K), about 200K, about 300K, about 400K, about 500K, about 600K, about 700K, about 800K, about 900K, about 1 million, about 2 million, about 3 million, about 4 million, about 5 million, or about 10 million.

### Epitope Mapping and Related Technologies

The epitope on the T-cell antigen (e.g., CD3) and/or the target antigen (e.g., a tumor-associated antigen) to which the antigen-binding molecules of the present disclosure bind may consist of a single contiguous sequence of 3 or more (e.g., 3, 4, 5, 6, 7, 8, 9, 10, 11, 12, 13, 14, 15, 16, 17, 18, 19, 20 or more) amino acids of a protein. Alternatively, the epitope may consist of a plurality of non-contiguous amino acids (or amino acid sequences) of the protein. The molecules of the disclosure may interact with, e.g., amino acids contained within a single CD3 chain (e.g., CD3-epsilon, CD3-delta or CD3-gamma), or may interact with amino acids on two or more different CD3 chains. The term “epitope,” as used herein, refers to an antigenic determinant that interacts with a specific antigen binding site in the variable region of an antigen-binding domain known as a paratope. A single antigen may have more than one epitope. Thus, different antigen-binding domains may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstances, an epitope may include moieties of saccharides, phosphoryl groups, or sulfonyl groups on the antigen.

Various techniques known to persons of ordinary skill in the art can be used to determine whether an antigen-binding domain of a molecule “interacts with one or more amino acids” within a polypeptide or protein. Exemplary techniques include, e.g., routine cross-blocking assay such as that described Antibodies, Harlow and Lane (Cold Spring Harbor Press, Cold Spring Harb., NY), alanine scanning mutational analysis, peptide blots analysis (Reineke, 2004, Methods Mol Biol 248:443-463), and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antigen-binding domain of a molecule interacts is hydrogen/deuterium exchange detected by mass spectrometry. In general terms, the hydrogen/deuterium exchange method involves deuterium-labeling the protein of interest, followed by binding the molecule to the deuterium-labeled protein. Next, the protein/molecule complex is transferred to water to allow hydrogen-deuterium exchange to occur at all residues except for the residues protected by the molecule (which remain deuterium-labeled). After dissociation of the molecule, the target protein is subjected to protease cleavage and mass spectrometry analysis, thereby revealing the deuterium-labeled residues which correspond to the specific amino acids with which the molecule interacts. See, e.g., Ehring (1999) Analytical Biochemistry 267(2):252-259; Engen and Smith (2001) Anal. Chem. 73:256A-265A. X-ray crystallography of the antigen/molecule complex may also be used for epitope mapping purposes.

### Bioequivalents

The present disclosure includes multispecific antigen-binding molecules that are bioequivalent to any of the exemplary multispecific antigen-binding molecules set forth herein. Two antigen-binding proteins are considered bioequivalent if, for example, they are pharmaceutical equivalents or pharmaceutical alternatives whose rate and extent of absorption do not show a significant difference when administered at the same molar dose under similar experimental conditions, either single dose or multiple doses. Some antigen-binding proteins will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antigen-binding protein or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antigen-binding protein (or its target) is measured as a function of time; and (d) in a well-controlled clinical trial that establishes safety, efficacy, or bioavailability or bioequivalence of an antigen-binding protein.

Bioequivalent variants of the exemplary multispecific antigen-binding molecules set forth herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antigen-binding proteins may include variants of the exemplary multispecific antigen-binding molecules set forth herein comprising amino acid changes which modify the glycosylation characteristics of the molecules, e.g., mutations which eliminate or remove glycosylation.

### Linkers

The multispecific antigen-binding molecules and polypeptides of the present disclosure comprise components that are connected to one another by a linker with one or more substrates for a protease and whose cleavage results in activation of the molecule or polypeptide.

A protease-cleavable linker can range from 2 amino acids to 80 or more amino acids, and in certain aspects a non-cleavable peptide linker ranges from 2 amino acids to 60 amino acids, 2 amino acids to 40 amino acids, from 2 amino acids to 50 amino acids, from 4 amino acids to 80 amino acids, or from 4 amino acids to 70 amino acids in length. In various embodiments, the protease-cleavable linker comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 amino acids.

The protease cleavable linkers comprise one or more substrate sequences for one or more proteases, for example one or more of the proteases discussed below. The one or more substrate sequences, e.g., one or more of the substrate sequences discussed below, are typically flanked by one or more spacer sequences, e.g., spacer sequences discussed below. Each protease-cleavable linker can include one, two, three or more substrate sequences. The spacer sequences can be adjoining, overlapping, or separated by spacer sequences. Preferably, the C- and N-termini of the protease-cleavable linkers contain spacer sequences.

Exemplary protease-cleavable linker sequences are set forth below in Table 8.

Exemplary proteases whose substrate sequences can be incorporated into the protease-cleavable linkers are set forth in Table 5 below.

TABLE 5
Exemplary Proteases for Substrate Cleavage

| | | |
|---|---|---|
| ADAMS, ADAMTS, e.g. | Caspases, e.g., | MMP24 |
| ADAM8 | Caspase 1 | MMP26 |
| ADAM9 | Caspase 2 | MMP27 |
| ADAM10 | Caspase 3 | |
| ADAM12 | Caspase 4 | |
| ADAM15 | Caspase 5 | |
| ADAM17/TACE | Caspase 6 | |
| ADAMDEC1 | Caspase 7 | |
| ADAMTS1 | Caspase 8 | Cysteine proteinases, e.g., |
| ADAMTS4 | Caspase 9 | Cruzipain |
| ADAMTS5 | Caspase 10 | Legumain |
| | Caspase 14 | Otubain-2 |
| Aspartate proteases, e.g., | | |
| BACE | Cysteine cathepsins, e.g., | KLKs, e.g., |
| Renin | Cathepsin B | KLK4 |
| | Cathepsin C | KLK5 |
| Aspartic cathepsins, e.g., | Cathepsin K | KLK6 |
| Cathepsin D | Cathepsin L | KLK7 |
| Cathepsin E | Cathepsin S | KLK8 |
| | Cathepsin V/L2 | KLK10 |
| NS3/4A | Cathepsin X/Z/P | KLK11 |
| PACE4 | | KLK13 |
| Plasmin | MMPs, e.g., | KLK14 |
| PSA | MMP1 | |
| tPA | MMP2 | Metallo proteinases, e.g., |
| Thrombin | MMP3 | Meprin |
| Tryptase | | Neprilysin |
| uPA | MMP7 | PSMA |
| | MMP8 | BMP-1 |
| Type II Transmembrane | MMP9 | |
| Serine Proteases (TTSPs), | MMP10 | |
| e.g., | | |
| DESC1 | MMP11 | Serine proteases, e.g., |
| DPP-4 | MMP12 | activated protein C |
| FAP | MMP13 | Cathepsin A |
| Hepsin | MMP14 | Cathepsin G |
| Matriptase-2 | MMP15 | Chymase |
| MT/SP1/Matriptase | MMP16 | coagulation factor proteases |
| | MMP17 | (e.g., FVIIa, FIXa, FXa, FXIa, |
| | | FXIIa) |
| TMPRSS2 | MMP19 | Human Neutrophil Elastase |
| TMPRSS3 | MMP20 | Lactoferrin |
| TMPRSS4 | MMP23 | |

In particular embodiments, the protease is matrix metalloprotease (MMP)-2, MMP-9, legumain asparaginyl endopeptidase, thrombin, fibroblast activation protease (FAP), MMP-1, MMP-3, MMP-7, MMP-8, MMP-12, MMP-13, MMP-14, membrane type 1 matrix metalloprotease (MT1-MMP), plasmin, transmembrane protease, serine (TMPRSS-3/4), cathepsin A, cathepsin B, cathepsin D, cathepsin E, cathepsin F, cathepsin H, cathepsin K, cathepsin L, cathepsin L2, cathepsin O, cathepsin S, caspase 1, caspase 2, caspase 3, caspase 4, caspase 5, caspase 6, caspase 7, caspase 8, caspase 9, caspase 10, caspase 11, caspase 12, caspase 13, caspase 14, human neutrophil elastase, urokinase/urokinase-type plasminogen activator (uPA), a disintegrin and metalloprotease (ADAM)10, ADAM12, ADAM17, ADAM with thrombospondin motifs (ADAMTS), ADAMTS5, beta secretase (BACE), granzyme A, granzyme B, guanidinobenzoatase, hepsin, matriptase, matriptase 2, meprin, neprilysin, prostate-specific membrane antigen (PSMA), tumor necrosis factor-converting enzyme (TACE), kallikrein-related peptidase (KLK)3, KLK5, KLK7, KLK11, NS3/4 protease of hepatitis C virus (HCV-NS3/4), tissue plasminogen activator (tPA), calpain, calpain 2, glutamate carboxypeptidase II, plasma kallikrein, AMSH-like protease, AMSH, γ-secretase component, antiplasmin cleaving enzyme (APCE), decysin 1, apoptosis-related cysteine peptidase, or N-acetylated alpha-linked acidic dipeptidase-like 1.

Exemplary substrate sequences that are cleavable by a tumor protease and can be incorporated into the protease-cleavable linkers are set forth in Table 6 below.

TABLE 6
Substrate Sequences for Protease Cleavable Linkers

| Substrate Sequence | Designation | Cleaving Protease |
|---|---|---|
| (DE)₈RPLALWRS(DR)₈ (SEQ ID NO: 169) | SU1 | MMP7 |
| AARGPAIH (SEQ ID NO: 170) | SU2 | |
| AAYHLVSQ (SEQ ID NO: 171) | SU3 | Collagenase |
| AGLGISST (SEQ ID NO: 172) | SU4 | Collagenase |
| AGLGVVER (SEQ ID NO: 173) | SU5 | Collagenase |
| ALAL (SEQ ID NO: 174) | SU6 | Lysosomal Enzyme |
| ALFFSSPP (SEQ ID NO: 175) | SU7 | |
| ALFKSSFP (SEQ ID NO: 176) | SU8 | |
| ALLLALL (SEQ ID NO: 177) | SU9 | TOP |
| AQFVLTEG (SEQ ID NO: 178) | SU10 | Collagenase |
| AQNLLGMV (SEQ ID NO: 179) | SU11 | |
| AVGLLAPP (SEQ ID NO: 180) | SU12 | Serine protease |
| DAFK (SEQ ID NO: 181) | SU13 | Urokinase plasminogen |
| | | activator (uPA) |
| DEVD (SEQ ID NO: 182) | SU14 | Caspase-3 |
| DEVDP (SEQ ID NO: 183) | SU15 | Caspase-3 |
| DPRSFL (SEQ ID NO: 184) | SU16 | Thrombin |
| DVAQFVLT (SEQ ID NO: 185) | SU17 | Collagenase |
| DVLK (SEQ ID NO: 186) | SU18 | Plasmin |
| DWLYWPGI (SEQ ID NO: 187) | SU19 | |
| EDDDDKA (SEQ ID NO: 188) | SU20 | Enterokinase |
| EP(Cit)G(Hof)YL (SEQ ID NO: 189) | SU21 | MMP2, MMP9, MMP14 |
| EPQALAMS (SEQ ID NO: 190) | SU22 | Collagenase |
| ESLPVVAV (SEQ ID NO: 191) | SU23 | Collagenase |
| ESPAYYTA (SEQ ID NO: 192) | SU24 | MMP |
| F(Pip)RS | SU25 | Thrombin |
| FK | SU26 | Lysosomal Enzyme |
| FPRPLGITGL (SEQ ID NO: 193) | SU27 | |
| FRLLDWQW (SEQ ID NO: 194) | SU28 | |
| GFLG (SEQ ID NO: 195) | SU29 | Lysosomal Enzyme |
| GGAANLVRGG (SEQ ID NO: 196) | SU30 | MMP11 |
| GGGRR (SEQ ID NO: 197) | SU31 | Urokinase plasminogen |
| | | activator (uPA) |
| GGPRGLPG (SEQ ID NO: 198) | SU32 | Cathepsin K |
| GGQPSGMWGW (SEQ ID NO: 199) | SU33 | |
| GGSIDGR (SEQ ID NO: 200) | SU34 | Factor Xa |
| GGWHTGRN (SEQ ID NO: 201) | SU35 | |
| GIAGQ (SEQ ID NO: 202) | SU36 | Collagenase |
| GKAFRR (SEQ ID NO: 203) | SU37 | Kallikrein 2 |
| GPAGLYAQ (SEQ ID NO: 204) | SU38 | |
| GPAGMKGL (SEQ ID NO: 205) | SU39 | |
| GPEGLRVG (SEQ ID NO: 206) | SU40 | Collagenase |
| GPLGIAGI (SEQ ID NO: 207) | SU41 | Collagenase |
| GPLGVRG (SEQ ID NO: 208) | SU42 | |
| GPQGIAGQ (SEQ ID NO: 209) | SU43 | Collagenase |
| GPQGLLGA (SEQ ID NO: 210) | SU44 | Collagenase |
| GPRSFG (SEQ ID NO: 211) | SU45 | |
| GPRSFGL (SEQ ID NO: 212) | SU46 | |
| GPSHLVLT (SEQ ID NO: 213) | SU47 | |
| GVSQNYPIVG (SEQ ID NO: 214) | SU48 | HIV Protease |
| GVVQASCRLA (SEQ ID NO: 215) | SU49 | CMV Protease |
| GWEHDG (SEQ ID NO: 216) | SU50 | Interleukin 1β converting |
| | | enzyme |
| HSSKLQ (SEQ ID NO: 217) | SU51 | Prostate Specific Antigen |
| HSSKLQEDA (SEQ ID NO: 218) | SU52 | Prostate Specific Antigen |
| HSSKLQL (SEQ ID NO: 219) | SU53 | Prostate Specific Antigen |
| HTGRSGAL (SEQ ID NO: 220) | SU54 | |
| IDGR (SEQ ID NO: 221) | SU55 | Factor Xa |
| IEGR (SEQ ID NO: 222) | SU56 | Factor Xa |
| ILPRSPAF (SEQ ID NO: 223) | SU57 | |
| IPVSLRSG (SEQ ID NO: 224) | SU58 | MMP |
| ISSGL (SEQ ID NO: 225) | SU59 | MMP |
| ISSGLL (SEQ ID NO: 226) | SU60 | MMP |
| ISSGLLS (SEQ ID NO: 227) | SU61 | MMP |
| ISSGLLSS (SEQ ID NO: 228) | SU62 | MMP |
| ISSGLSS (SEQ ID NO: 229) | SU63 | MMP |
| KGSGDVEG (SEQ ID NO: 230) | SU64 | Caspase-3 |
| KQEQNPGST (SEQ ID NO: 231) | SU65 | FAP |
| KRALGLPG (SEQ ID NO: 232) | SU66 | MMP7 |
| LAAPLGLL (SEQ ID NO: 233) | SU67 | |
| LAPLGLQRR (SEQ ID NO: 234) | SU68 | |
| LAQKLKSS (SEQ ID NO: 235) | SU69 | |
| LAQRLRSS (SEQ ID NO: 236) | SU70 | |
| LEATA (SEQ ID NO: 237) | SU71 | MMP9 |
| LKAAPRWA (SEQ ID NO: 238) | SU72 | |
| LLAPSHRA (SEQ ID NO: 239) | SU73 | |
| LPGGLSPW (SEQ ID NO: 240) | SU74 | |
| LSGRSANI (SEQ ID NO: 241) | SU75 | Serine protease |
| LSGRSANP (SEQ ID NO: 242) | SU76 | Serine protease |
| LSGRSDDH (SEQ ID NO: 243) | SU77 | Serine protease |
| LSGRSDIH (SEQ ID NO: 244) | SU78 | Serine protease |
| LSGRSDNH (SEQ ID NO: 245) | SU79 | Serine protease |
| LSGRSDNI (SEQ ID NO: 246) | SU80 | Serine protease |
| LSGRSDNP (SEQ ID NO: 247) | SU81 | Serine protease |
| LSGRSDQG (SEQ ID NO: 248) | SU82 | Serine protease |
| LSGRSDQH (SEQ ID NO: 249) | SU83 | Serine protease |
| LSGRSDTH (SEQ ID NO: 250) | SU84 | Serine protease |
| LSGRSDYH (SEQ ID NO: 251) | SU85 | Serine protease |
| LSGRSGNH (SEQ ID NO: 252) | SU86 | Serine protease |
| LVLASSSFGY (SEQ ID NO: 253) | SU87 | Herpes Simplex Virus |
| | | Protease |
| MDAFLESS (SEQ ID NO: 254) | SU88 | Collagenase |
| MGLFSEAG (SEQ ID NO: 255) | SU89 | |
| MIAPVAYR (SEQ ID NO: 256) | SU90 | |
| MVLGRSLL (SEQ ID NO: 257) | SU91 | |
| NLL | SU92 | Cathepsin B |
| NTLSGRSENHSG (SEQ ID NO: 258) | SU93 | |
| NTLSGRSGNHGS (SEQ ID NO: 259) | SU94 | |
| PAGLWLDP (SEQ ID NO: 260) | SU95 | |
| PGGPAGIG (SEQ ID NO: 261) | SU96 | |
| PIC(Et)FF (SEQ ID NO: 262) | SU97 | Cathepsin D |
| PLGC(me)AG (SEQ ID NO: 263) | SU98 | MMP |
| PLGL (SEQ ID NO: 264) | SU99 | |
| PLGLAG (SEQ ID NO: 265) | SU100 | MMP |
| PLGLAX (SEQ ID NO: 266) | SU101 | MMP |
| PLGLWA (SEQ ID NO: 267) | SU102 | MMP |
| PLGLWSQ (SEQ ID NO: 268) | SU103 | MMP |
| PLTGRSGG (SEQ ID NO: 269) | SU104 | |
| PMAKK (SEQ ID NO: 270) | SU105 | |
| PPRSFL (SEQ ID NO: 1271) | SU106 | Thrombin |
| PR(S/T)(L/I)(S/T) | SU107 | MMP9 |
| PRFRIIGG (SEQ ID NO: 272) | SU108 | Plasmin |
| PVGYTSSL (SEQ ID NO: 273) | SU109 | |
| PVQPIGPQ (SEQ ID NO: 274) | SU110 | Collagenase |
| QALAMSAI (SEQ ID NO: 275) | SU111 | Collagenase |
| QGRAITFI (SEQ ID NO: 276) | SU112 | |
| QNQALRMA (SEQ ID NO: 277) | SU113 | |
| RGPA (SEQ ID NO: 278) | SU114 | |
| RGPAFNPM (SEQ ID NO: 279) | SU115 | |
| RGPATPIM (SEQ ID NO: 280) | SU116 | |
| RKSSIIIRMRDVVL (SEQ ID NO: 281) | SU117 | Plasmin |
| RLQLKAC (SEQ ID NO: 282) | SU118 | MMP |
| RLQLKL (SEQ ID NO: 283) | SU119 | MMP |
| RMHLRSLG (SEQ ID NO: 284) | SU120 | |
| RPSPMWAY (SEQ ID NO: 285) | SU121 | |
| RQARVVNG (SEQ ID NO: 286) | SU122 | Matripase |
| SAGFSLPA (SEQ ID NO: 287) | SU123 | |
| SAPAVESE (SEQ ID NO: 288) | SU124 | Collagenase |
| SARGPSRW (SEQ ID NO: 289) | SU125 | |
| SGEPAYYTA (SEQ ID NO: 290) | SU126 | |
| SGGPLGVR (SEQ ID NO: 291) | SU127 | |
| SGRIGFLRTA (SEQ ID NO: 292) | SU128 | MMP14 |
| SGRSA (SEQ ID NO: 293) | SU129 | Urokinase plasminogen |
| | | activator (uPA) |
| SGRSANPRG (SEQ ID NO: 294) | SU130 | |
| SMLRSMPL (SEQ ID NO: 295) | SU131 | |
| SPLPLRVP (SEQ ID NO: 296) | SU132 | |
| SPLTGRSG (SEQ ID NO: 297) | SU133 | |
| SPRSIMLA (SEQ ID NO: 298) | SU134 | |
| SSRGPAYL (SEQ ID NO: 299) | SU135 | |
| SSRHRRALD (SEQ ID NO: 300) | SU136 | Plasmin |
| SSSFDKGKYKKGDDA (SEQ ID NO: 301) | SU137 | Plasmin |
| SSSFDKGKYKRGDDA (SEQ ID NO: 302) | SU138 | Plasmin |
| STFPFGMF (SEQ ID NO: 303) | SU139 | |
| TARGPSFK (SEQ ID NO: 304) | SU140 | |
| TGRGPSWV (SEQ ID NO: 305) | SU141 | |
| TSGRSANP (SEQ ID NO: 306) | SU142 | |
| TSTSGRSANPRG (SEQ ID NO: 307) | SU143 | |
| VAGRSMRP (SEQ ID NO: 308) | SU144 | |
| VAQFVLTE (SEQ ID NO: 309) | SU145 | Collagenase |
| VHMPLGFLGP (SEQ ID NO: 310) | SU146 | |
| VPLSLYSG (SEQ ID NO: 311) | SU147 | MMP9 |
| VVPEGRRS (SEQ ID NO: 312) | SU148 | |
| WATPRPMR (SEQ ID NO: 313) | SU149 | |
| YGAGLGVV (SEQ ID NO: 314) | SU150 | Collagenase |
| HPVGLLAR (SEQ ID NO: 423) | SU151 | |

Exemplary spacer sequences that can be incorporated into the protease-cleavable linkers are set forth in Table 7 below. In addition to the spacer sequences set forth in Table 7, any of the non-cleavable linker sequences described below, e.g., the non-cleavable linker sequences set forth in Table 9, or portions thereof can be used as spacer sequences.

TABLE 7
Spacer Sequences for Protease Cleavable Linkers

| Spacer Sequence | Designation |
|---|---|
| (GGGGS)ₙ (SEQ ID NO: 315) | SP1 |
| (GGGS)ₙ (SEQ ID NO: 316) | SP2 |
| (GGS)ₙ | SP3 |
| (GS)ₙ | SP4 |
| (GSGGS)ₙ (SEQ ID NO: 317) | SP5 |
| GGGGSGGGGS (SEQ ID NO: 318) | SP6 |
| GGGGSGGGGSGGGGS (SEQ ID NO: 319) | SP7 |
| GGGGSGGGGSGGGGSGGGGS (SEQ ID NO: 320) | SP8 |
| GGGKSGGGKSGGGKS (SEQ ID NO: 321) | SP9 |
| GGGKSGGKGSGKGGS (SEQ ID NO: 322) | SP10 |
| GGGS (SEQ ID NO: 323) | SP11 |
| GGGSG (SEQ ID NO: 324) | SP12 |
| GGKGSGGKGSGGKGS (SEQ ID NO: 325) | SP13 |
| GGSGGGGSGGGGS (SEQ ID NO: 326) | SP14 |
| GGSGGS (SEQ ID NO: 327) | SP15 |
| GGSGGSGGSGS (SEQ ID NO: 328) | SP16 |
| GSGGG (SEQ ID NO: 329) | SP17 |
| GSGSG (SEQ ID NO: 330) | SP18 |
| GSS | SP19 |
| GSSG (SEQ ID NO: 331) | SP20 |
| GSSGGSGGSG (SEQ ID NO: 332) | SP21 |
| GSSGGSGGSGG (SEQ ID NO: 333) | SP22 |
| GSSGGSGGSGGS (SEQ ID NO: 334) | SP23 |
| GSSGGSGGSGGSG (SEQ ID NO: 335) | SP24 |
| GSSGGSGGSGGSGGGS (SEQ ID NO: 336) | SP25 |
| GSSGGSGGSGS (SEQ ID NO: 337) | SP26 |
| GSSGT (SEQ ID NO: 338) | SP27 |
| GSSSG (SEQ ID NO: 339) | SP28 |
| QGQSGQ (SEQ ID NO: 340) | SP29 |
| QGQSGQG (SEQ ID NO: 341) | SP30 |
| QGQSGS (SEQ ID NO: 342) | SP31 |
| QSGQ (SEQ ID NO: 343) | SP32 |
| QSGQG (SEQ ID NO: 344) | SP33 |
| QSGS (SEQ ID NO: 345) | SP34 |
| SGQ | SP35 |
| SGQG (SEQ ID NO: 346) | SP36 |
| SGS | SP37 |
| (G)ₙ | SP38 |

In some embodiments, as used in Table 7 above, n is an integer from 1 to 10, e.g., 1, 2, 3, 4, 5, 6, 7, 8, 9 or 10.

Exemplary protease-cleavable linkers comprising one or more substrate sequences as well as spacer sequences are set forth in Table 8 below.

TABLE 8
Protease Cleavable Linker Sequences

| Linker Sequence | Designation | Cleaving Protease(s) |
|---|---|---|
| GGGISSGLLSGRSDNHGGGISSG | PCL1 | |
| LLSGRSDNHGGS (SEQ ID NO: | | |
| 347) | | |
| GGGISSGLLSGRSDNHGGGISSG | PCL2 | |
| LLSGRSDNHGGS | | |
| GGGISSGLLSGRSDNHGGGISSG | | |
| LLSGRSDNHGGS (SEQ ID NO: | | |
| 348) | | |
| GGSGGSIPVSLRSGGGISSGLLS | PCL3 | |
| GRSDNHGGSGGS (SEQ ID NO: | | |
| 349) | | |
| GGSGGSVPLSLYSGGGISSGLLS | PCL4 | |
| GRSDNHGGSGGS (SEQ ID NO: | | |
| 350) | | |
| GGSHPVGLLARGGGHPVGLLAR | PCL5 | |
| GGGHPVGLLARGS (SEQ ID NO: | | |
| 351) | | |
| GGSHPVGLLARGGGHPVGLLAR | PCL6 | |
| GGSGRSAGGSGRSA (SEQ ID | | |
| NO: 352) | | |
| AVGLLAPPGGLSGRSANI (SEQ ID | PCL7 | ADAM17_2, FAPa_1, CTSL1_1 |
| NO: 353) | | |
| AVGLLAPPGGLSGRSANP (SEQ | PCL8 | FAPa_1, ADAM17_2, CTSL1_1 |
| ID NO: 354) | | |
| AVGLLAPPGGLSGRSDDH (SEQ | PCL9 | MMP14_1, MMP14_1, MMP14_1 |
| ID NO: 355) | | |
| AVGLLAPPGGLSGRSDIH (SEQ | PCL10 | MMP14_1, MMP14_1, MMP14_1 |
| ID NO: 356) | | |
| AVGLLAPPGGLSGRSDNH (SEQ | PCL11 | MMP14_1, MMP14_1 |
| ID NO: 357) | | |
| AVGLLAPPGGLSGRSDNI (SEQ | PCL12 | MMP14_1, CTSL1_1, ADAM17_2 |
| ID NO: 358) | | |
| AVGLLAPPGGLSGRSDNP (SEQ | PCL13 | CTSL1_1, ADAM17_2, FAPa_1 |
| ID NO: 359) | | |
| AVGLLAPPGGLSGRSDQH (SEQ | PCL14 | |
| ID NO: 360) | | |
| AVGLLAPPGGLSGRSDTH (SEQ | PCL15 | FAPa_1, CTSL1_1, ADAM17_2 |
| ID NO: 361) | | |
| AVGLLAPPGGLSGRSDYH (SEQ | PCL16 | |
| ID NO: 362) | | |
| AVGLLAPPGGTSTSGRSANPRG | PCL17 | |
| (SEQ ID NO: 363) | | |
| AVGLLAPPSGRSANPRG (SEQ ID | PCL18 | |
| NO: 364) | | |
| AVGLLAPPTSGRSANPRG (SEQ | PCL19 | |
| ID NO: 365) | | |
| GGALFKSSFPGPAGLYAQPLAQK | PCL20 | CTSL1_1, MMP14_1, ADAM17_2 |
| LKSSGGK (SEQ ID NO: 366) | | |
| GGGGSGGGGSGGGGSFVGGTG | PCL21 | |
| GGGSGGGGSGGS (SEQ ID NO: | | |
| 367) | | |
| GGGGSGGGGSGGGGSISSGLLS | PCL22 | |
| GRSDNHGGSGGS (SEQ ID NO: | | |
| 368) | | |
| GGGGSGGGGSGGGGSVPLSLYS | PCL23 | |
| GGGSGGSGGSGS (SEQ ID NO: | | |
| 369) | | |
| GGGGSGGGGSGPLGLWSQGGG | PCL24 | |
| GSGGGGSGGGGSGG (SEQ ID | | |
| NO: 370) | | |
| GGGGSGGGGSKKAAPGGGGSG | PCL25 | |
| GGGSGGGGSGGS (SEQ ID NO: | | |
| 371) | | |
| GGGGSGGGGSKKAAPVNGGGG | PCL26 | |
| GSGGGGSGGGGS (SEQ ID NO: | | |
| 372) | | |
| GGGGSGGGGSPMAKKGGGGSG | PCL27 | |
| GGGSGGGGSGGS (SEQ ID NO: | | |
| 373) | | |
| GGGGSGGGGSPMAKKVNGGGG | PCL28 | |
| GSGGGGSGGGGS (SEQ ID NO: | | |
| 374) | | |
| GGGGSGGGGSQARAKGGGGSG | PCL29 | |
| GGGSGGGGSGGS (SEQ ID NO: | | |
| 375) | | |
| GGGGSGGGGSQARAKVNGGGG | PCL30 | |
| GSGGGGSGGGGS (SEQ ID NO: | | |
| 376) | | |
| GGGGSGGGGSRQARVVNGGGG | PCL31 | |
| GSGGGGSGGGGS (SEQ ID NO: | | |
| 377) | | |
| GGGGSGGGGSRQARVVNGGGG | PCL32 | |
| GSVPLSLYSGGGGGSGGGGS | | |
| (SEQ ID NO: 378) | | |
| GGGGSGGGGSRQARVVNSVPLS | PCL33 | |
| LYSGGGGGSGGGGS (SEQ ID | | |
| NO: 379) | | |
| GGGGSGGGGSVHMPLGFLGPG | PCL34 | |
| GGGSGGGGSGGS (SEQ ID NO: | | |
| 380) | | |
| GGGGSVHMPLGFLGPGRSRGSF | PCL35 | |
| PGGGGS (SEQ ID NO: 381) | | |
| GGGGSVHMPLGFLGPPMAKKGG | PCL36 | |
| GGSGGGGSGGS (SEQ ID NO: | | |
| 382) | | |
| GGGGSVHMPLGFLGPRQARVVN | PCL37 | |
| GGGGSGGGGS (SEQ ID NO: | | |
| 383) | | |
| GGGGSVHMPLGFLGPRQARVVN | PCL38 | |
| GGGGSGGGGSGG (SEQ ID NO: | | |
| 384) | | |
| GGPLAQKLKSSALFKSSFPGPAG | PCL39 | ADAM17_2, CTSL1_1, MMP14_1 |
| LYAQGGR (SEQ ID NO: 385) | | |
| GLSGRSDNHGGAVGLLAPP | PCL40 | |
| (SEQ ID NO: 386) | | |
| GLSGRSDNHGGVHMPLGFLGP | PCL41 | |
| (SEQ ID NO: 387) | | |
| ISSGLLSGRSANI (SEQ ID NO: | PCL42 | MMP, Serine protease |
| 388) | | |
| ISSGLLSGRSANP (SEQ ID NO: | PCL43 | MMP, Serine protease |
| 389) | | |
| ISSGLLSGRSANPRG (SEQ ID | PCL44 | MMP, Serine protease |
| NO: 390) | | |
| ISSGLLSGRSDDH (SEQ ID NO: | PCL45 | MMP, Serine protease |
| 391) | | |
| ISSGLLSGRSDIH (SEQ ID NO: | PCL46 | MMP, Serine protease |
| 392) | | |
| ISSGLLSGRSDNH (SEQ ID NO: | PCL47 | MMP, Serine protease |
| 393) | | |
| ISSGLLSGRSDNI (SEQ ID NO: | PCL48 | CTSL1_1, MMP14_1 |
| 394) | | |
| ISSGLLSGRSDNP (SEQ ID NO: | PCL49 | MMP, Serine protease |
| 395) | | |
| ISSGLLSGRSDQH (SEQ ID NO: | PCL50 | MMP, Serine protease |
| 396) | | |
| ISSGLLSGRSDTH (SEQ ID NO: | PCL51 | MMP, Serine protease |
| 397) | | |
| ISSGLLSGRSDYH (SEQ ID NO: | PCL52 | MMP, Serine protease |
| 398) | | |
| ISSGLLSGRSGNH (SEQ ID NO: | PCL53 | MMP, Serine protease |
| 399) | | |
| ISSGLLSSGGSGGSLSGRSDNH | PCL54 | |
| (SEQ ID NO: 400) | | |
| ISSGLLSSGGSGGSLSGRSGNH | PCL55 | |
| (SEQ ID NO: 401) | | |
| KGGPGGPAGIGPLAQRLRSSALF | PCL56 | FAPa_1, ADAM17_1, CTSL1_1 |
| KSSFPGR (SEQ ID NO: 402) | | |
| KSGPGGPAGIGALFFSSPPLAQKL | PCL57 | FAPa_1, CTSL1_2, ADAM17_2 |
| KSSGGR (SEQ ID NO: 403) | | |
| LSGRSDNHGGAVGLLAPP (SEQ | PCL58 | |
| ID NO: 404) | | |
| LSGRSDNHGGSGGSISSGLLSS | PCL59 | |
| (SEQ ID NO: 405) | | |
| LSGRSDNHGGSGGSQNQALRMA | PCL60 | |
| (SEQ ID NO: 406) | | |
| LSGRSDNHGGVHMPLGFLGP | PCL61 | |
| (SEQ ID NO: 407) | | |
| LSGRSGNHGGSGGSISSGLLSS | PCL62 | |
| (SEQ ID NO: 408) | | |
| LSGRSGNHGGSGGSQNQALRMA | PCL63 | |
| (SEQ ID NO: 409) | | |
| QNQALRMAGGSGGSLSGRSDNH | PCL64 | |
| (SEQ ID NO: 410) | | |
| QNQALRMAGGSGGSLSGRSGNH | PCL65 | |
| (SEQ ID NO: 411) | | |
| RGGALFKSSFPLAQKLKSSGPAG | PCL66 | CTSL1_1, ADAM17_2, MMP14_1 |
| LYAQGGK (SEQ ID NO: 412) | | |
| RGGGPAGLYAQPLAQKLKSSALF | PCL67 | MMP14_1, ADAM17_2, CTSL1_1 |
| KSSFPGG (SEQ ID NO: 413) | | |
| SGGFPRSGGSFNPRTFGSKRKR | PCL68 | thrombin, factor Xa, hepsin |
| RGSRGGGG (SEQ ID NO: 414) | | |
| SGPLAQKLKSSGPAGLYAQALFK | PCL69 | ADAM17_2, MMP14_1, CTSL1_1 |
| SSFPGSK (SEQ ID NO: 415) | | |
| TSTSGRSANPRGGGAVGLLAPP | PCL70 | |
| (SEQ ID NO: 416) | | |
| TSTSGRSANPRGGGVHMPLGFL | PCL71 | |
| GP (SEQ ID NO: 417) | | |
| VHMPLGFLGPGGLSGRSDNH | PCL72 | |
| (SEQ ID NO: 418) | | |
| VHMPLGFLGPGGTSTSGRSANP | PCL73 | |
| RG (SEQ ID NO: 419) | | |
| SGRSAGGGSGRSAGGGSGRSA | PCL74 | uPA |
| (SEQ ID NO: 420) | | |
| HPVGLLARGGGHPVGLLARGGG | PCL75 | MPA (MMP-2 and uPA) |
| SGRSAGGGSGRSA (SEQ ID NO: | | |
| 421) | | |
| GPLGVRGK (SEQ ID NO: 422) | PCL76 | MMP-2 |
| HPVGLLAR (SEQ ID NO: 423) | PCL77 | MMP-2 |
| GPQGIAGQ (SEQ ID NO: 209) | PCL78 | MMP-2, MMP-9, and to some degree |
| | | MT1-MMP |
| VPMSMRGG (SEQ ID NO: 424) | PCL79 | MMP-9 and MMP-2 |
| IPVSLRSG (SEQ ID NO: 224) | PCL80 | MMP-2, and to some degree MMP-9 or |
| | | MMP-7 |
| RPFSMIMG (SEQ ID NO: 425) | PCL81 | MMP-9 and MMP-7, to some degree |
| | | MMP-3 |
| VPLSLTMG (SEQ ID NO: 426) | PCL82 | MMP-7, to some degree MMP-9, MMP- |
| | | 2, MPT-1-MMP |
| VPLSLYSG (SEQ ID NO: 311) | PCL83 | MMP-2, MMP-9, MMP-7 |
| IPESLRAG (SEQ ID NO: 427) | PCL84 | MMP-2, MMP-7, MMP-9, to some |
| | | degree MPT-1-MMP |
| GGGISSGLLSGRSDNHGGGS | PCL85 | |
| (SEQ ID NO: 478) | | |
| GGGHPVGLLARGGGS (SEQ ID | PCL86 | |
| NO: 479) | | |
| GGGSGGGSGGGGISSGLLSGRS | PCL87 | |
| DNHGGGSGGGSGGS (SEQ ID | | |
| NO: 480) | | |
| GGGGISSGLLSGRSDNHGGGISS | PCL88 | |
| GLLSGRSDNHGGS (SEQ ID NO: | | |
| 481) | | |
| GGGSGGSIPVSLRSGGGISSGLL | PCL89 | |
| SGRSDNHGGSGGS (SEQ ID NO: | | |
| 482) | | |
| GGGSGGSVPLSLYSGGGISSGLL | PCL90 | |
| SGRSDNHGGSGGS (SEQ ID NO: | | |
| 483) | | |
| GGGSHPVGLLARGGGHPVGLLA | PCL91 | |
| RGGGHPVGLLARGS (SEQ ID | | |
| NO: 484) | | |
| GGGSHPVGLLARGGGHPVGLLA | PCL92 | |
| RGGSGRSAGGSGRS (SEQ ID | | |
| NO: 485) | | |

In certain aspects, the protease-cleavable linker comprises an amino acid sequence having up to 5, up to 4, up to 3, up to 2 or up to 1 amino acid substitution(s) as compared to the sequence set forth in Table 8. Thus, in some embodiments, the protease cleavable linker comprises or consists of any amino acid sequence in Table 8 with 1-5 amino acid substitutions as compared to the sequence set forth in Table 8.

In certain aspects, the present disclosure provides multispecific antigen-binding molecules and polypeptides in which two or more components are connected to one another by a linker.

Preferably, all linkers in the molecules or polypeptides other than the protease-cleavable linker whose cleavage results in activation of the molecules or polypeptides are non-cleavable linkers (NCLs).

A non-cleavable linker can range from 2 amino acids to 60 or more amino acids, and in certain aspects a non-cleavable peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length.

In particular aspects, a non-cleavable linker is at least 5 amino acids, at least 6 amino acids or at least 7 amino acids in length and optionally is up to 30 amino acids, up to 40 amino acids, up to 50 amino acids or up to 60 amino acids in length.

In some embodiments of the foregoing, the non-cleavable linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the non-cleavable linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments of the foregoing, the non-cleavable linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

Charged (e.g., charged hydrophilic linkers) and/or flexible non-cleavable linkers may be used. Examples of flexible non-cleavable linkers that can be used in the molecules and polypeptides discussed herein include those disclosed by Chen et al., 2013, Adv Drug Deliv Rev. 65(10): 1357-1369 and Klein et al., 2014, Protein Engineering, Design & Selection 27(10): 325-330. Particularly useful flexible non-cleavable linkers are or comprise repeats of glycines and serines, e.g., a monomer or multimer of GnS or SGn, where n is an integer from 1 to 10, e.g., 1 2, 3, 4, 5, 6, 7, 8, 9 or 10. Other exemplary flexible linkers include glycine polymers (G)n, glycine-serine polymers (GS)ₙ, where n is an integer of at least one (e.g., from 1-20), glycine-alanine polymers, alanine-serine polymers, and other flexible linkers known in the art. Specific linkers include (G₄S)ₙ linkers, wherein n=1-10, or n is 1, 2, 3, 4, 5, 6, 7, 8, 9, or 10.

Exemplary non-cleavable linker sequences are set forth in Table 9 below.

TABLE 9
Non-Cleavable Linker Sequences

| | Designa- |
|---|---|
| Linker Sequence | tion |
| (GGGGS)ₙ (SEQ ID NO: 315) | NCL1 |
| (GGGS)ₙ (SEQ ID NO: 316) | NCL2 |
| (GGS)ₙ | NCL3 |
| (GS)ₙ | NCL4 |
| (GSGGS)ₙ (SEQ ID NO: 317) | NCL5 |
| ADAAP (SEQ ID NO: 428) | NCL6 |
| ADAAPTVSIFP (SEQ ID NO: 429) | NCL7 |
| ADAAPTVSIFPP (SEQ ID NO: 430) | NCL8 |
| AKTTAP (SEQ ID NO: 431) | NCL9 |
| AKTTAPSVYPLAP (SEQ ID NO: 432) | NCL10 |
| AKTTPKLEEGEFSEARV (SEQ ID NO: 433) | NCL11 |
| AKTTPKLGG (SEQ ID NO: 434) | NCL12 |
| AKTTPP (SEQ ID NO: 435) | NCL13 |
| AKTTPPSVTPLAP (SEQ ID NO: 436) | NCL14 |
| ASTKGP (SEQ ID NO: 437) | NCL15 |
| ASTKGPSVFPLAPASTKGPSVFPLAP (SEQ ID NO: | NCL16 |
| 438) | |
| EGKSSGSGSESKST (SEQ ID NO: 439) | NCL17 |
| GEGESGEGESGEGES (SEQ ID NO: 440) | NCL18 |
| GEGESGEGESGEGESGEGES (SEQ ID NO: 441) | NCL19 |
| GEGGSGEGGSGEGGS (SEQ ID NO: 442) | NCL20 |
| GENKVEYAPALMALS (SEQ ID NO: 443) | NCL21 |
| GGEGSGGEGSGGEGS (SEQ ID NO: 444) | NCL22 |
| GGGESGGEGSGEGGS (SEQ ID NO: 445) | NCL23 |
| GGGESGGGESGGGES (SEQ ID NO: 446) | NCL24 |
| GGGGSGGGGS (SEQ ID NO: 318) | NCL25 |
| GGGGSGGGGSGGGGS (SEQ ID NO: 319) | NCL26 |
| GGGGSGGGGSGGGGGGGGS (SEQ ID NO: 320) | NCL27 |
| GGGKSGGGKSGGGKS (SEQ ID NO: 321) | NCL28 |
| GGGKSGGKGSGKGGS (SEQ ID NO: 322) | NCL29 |
| GGGS (SEQ ID NO: 323) | NCL30 |
| GGGSG (SEQ ID NO: 324) | NCL31 |
| GGKGSGGKGSGGKGS (SEQ ID NO: 325) | NCL32 |
| GGS | NCL33 |
| GGSG (SEQ ID NO: 447) | NCL34 |
| GGSGG (SEQ ID NO: 448) | NCL35 |
| GGSGG (SEQ ID NO: 448) | NCL36 |
| GGSGGGGSG (SEQ ID NO: 449) | NCL37 |
| GGSGGGGSGGGGS (SEQ ID NO: 326) | NCL38 |
| GHEAAAVMQVQYPAS (SEQ ID NO: 450) | NCL39 |
| GKGGSGKGGSGKGGS (SEQ ID NO: 451) | NCL40 |
| GKGKSGKGKSGKGKS (SEQ ID NO: 452) | NCL41 |
| GKGKSGKGKSGKGKSGKGKS (SEQ ID NO: 453) | NCL42 |
| GKPGSGKPGSGKPGS (SEQ ID NO: 454) | NCL43 |
| GKPGSGKPGSGKPGSGKPSGS (SEQ ID NO: 455) | NCL44 |
| GPAKELTPLKEAKVS (SEQ ID NO: 456) | NCL45 |
| GSAGSAAGSGEF (SEQ ID NO: 457) | NCL46 |
| GSGGG (SEQ ID NO: 329) | NCL47 |
| GSGSG (SEQ ID NO: 330) | NCL48 |
| GSS | NCL49 |
| GSSG (SEQ ID NO: 331) | NCL50 |
| GSSGGSGGSG (SEQ ID NO: 332) | NCL51 |
| GSSGGSGGSGG (SEQ ID NO: 333) | NCL52 |
| GSSGGSGGSGGS (SEQ ID NO: 334) | NCL53 |
| GSSGGSGGSGGSG (SEQ ID NO: 335) | NCL54 |
| GSSGGSGGSGGSGGGS (SEQ ID NO: 336) | NCL55 |
| GSSGGSGGSGS (SEQ ID NO: 337) | NCL56 |
| GSSGT (SEQ ID NO: 338) | NCL57 |
| GSSSG (SEQ ID NO: 339) | NCL58 |
| GSTSGSGKPGSGEGSTKG (SEQ ID NO: 458) | NCL59 |
| GTAAAGAGAAGGAAAGAAG (SEQ ID NO: 459) | NCL60 |
| GTSGSSGSGSGGSGSGGGG (SEQ ID NO: 460) | NCL61 |
| IRPRAIGGSKPRVA (SEQ ID NO: 461) | NCL62 |
| KESGSVSSEQLAQFRSLD (SEQ ID NO: 462) | NCL63 |
| KTTPKLEEGEFSEAR (SEQ ID NO: 463) | NCL64 |
| PRGASKSGSASQTGSAPGS (SEQ ID NO: 464) | NCL65 |
| QPKAAP (SEQ ID NO: 465) | NCL66 |
| QPKAAPSVTLFPP (SEQ ID NO: 466) | NCL67 |
| RADAAAA(G4S)₄ (SEQ ID NO: 467) | NCL68 |
| RADAAAAGGPGS (SEQ ID NO: 468) | NCL69 |
| RADAAP (SEQ ID NO: 469) | NCL70 |
| RADAAPTVS (SEQ ID NO: 470) | NCL71 |
| SAKTTP (SEQ ID NO: 471) | NCL72 |
| SAKTTPKLEEGEFSEARV (SEQ ID NO: 472) | NCL73 |
| SAKTTPKLGG (SEQ ID NO: 473) | NCL74 |
| STAGDTHLGGEDFD (SEQ ID NO: 474) | NCL75 |
| TVAAP (SEQ ID NO: 475) | NCL76 |
| TVAAPSVFIFPP (SEQ ID NO: 476) | NCL77 |
| TVAAPSVFIFPPTVAAPSVFIFPP (SEQ ID NO: | NCL78 |
| 477) | |

### Therapeutic Uses of the Antigen-Binding Molecules

The present disclosure includes methods comprising administering to a subject in need thereof a therapeutic composition comprising a multispecific antigen-binding molecule or polypeptide that specifically binds a T-cell antigen (e.g., CD3) and a target antigen (e.g., a tumor-associated antigen). The therapeutic composition can comprise any of the multispecific antigen-binding molecules as disclosed herein and a pharmaceutically acceptable carrier or diluent. As used herein, the expression “a subject in need thereof” means a human or non-human animal that exhibits one or more symptoms or indicia of cancer, or who otherwise would benefit from an inhibition or reduction in target antigen activity or a depletion of target-antigen positive cells (e.g., tumor cells).

The multispecific antigen-binding molecules of the disclosure (and therapeutic compositions comprising the same) are useful, inter alia, for treating any disease or disorder in which stimulation, activation and/or targeting of an immune response would be beneficial. In particular, the multispecific antigen-binding molecules of the present disclosure may be used for the treatment, prevention and/or amelioration of any disease or disorder associated with or mediated by target antigen expression or activity or the proliferation of target-antigen positive cells. The mechanism of action by which the therapeutic methods of the disclosure are achieved includes killing of the cells expressing the target antigen in the presence of T cells.

The multispecific antigen-binding molecules and polypeptides of the present disclosure may be used to treat a disease or disorder associated with target antigen expression including, e.g., a cancer. Analytic/diagnostic methods known in the art, such as tumor scanning, etc., may be used to ascertain whether a patient harbors a tumor cell that is positive for the target antigen. In some cases, the cancer is selected from a solid tumor, cervical cancer, head and neck squamous cell carcinoma, melanoma, prostate cancer, acute myeloid leukemia, pancreatic cancer, colon cancer, acute lymphocytic leukemia, a non-Hodgkin's lymphoma, gastric cancer, post-transplant lymphoproliferative disorder, ovarian cancer, lung cancer, squamous cell carcinoma, non-small cell lung cancer esophageal cancer, bladder cancer, nasopharyngeal cancer, uterine cancer, liver cancer, testicular cancer, or breast cancer.

The present disclosure also includes methods for treating residual cancer in a subject. As used herein, the term “residual cancer” means the existence or persistence of one or more cancerous cells in a subject following treatment with an anti-cancer therapy.

According to certain aspects, the present disclosure provides methods for treating a disease or disorder associated with target antigen expression (e.g., a cancer or infection) comprising administering one or more of the multispecific antigen-binding molecules or polypeptides described elsewhere herein to a subject after the subject has been determined to have a target antigen positive cancer.

### Combination Therapies

The present disclosure provides methods which comprise administering a pharmaceutical composition comprising any of the exemplary multispecific antigen-binding molecules or polypeptides described herein in combination with one or more additional therapeutic agents. Exemplary additional therapeutic agents that may be combined with or administered in combination with an antigen-binding molecule of the present disclosure include, e.g., an anti-tumor agent (e.g. chemotherapeutic agents). In certain embodiments, the second therapeutic agent may be a monoclonal antibody, an antibody drug conjugate, a bispecific antibody conjugated to an anti-tumor agent, a checkpoint inhibitor, or combinations thereof. Other agents that may be beneficially administered in combination with the antigen-binding molecules of the disclosure include cytokine inhibitors, including small-molecule cytokine inhibitors and antibodies that bind to cytokines such as IL-1, IL-2, IL-3, IL-4, IL-5, IL-6, IL-8, IL-9, IL-10, IL-11, IL-12, IL-13, IL-17, IL-18, or to their respective receptors. The pharmaceutical compositions of the present disclosure (e.g., pharmaceutical compositions comprising a multispecific antigen-binding molecule as disclosed herein) may also be administered as part of a therapeutic regimen comprising one or more therapeutic combinations selected from a monoclonal antibody that may interact with a different antigen on the cell surface, a bispecific antibody that has one arm that binds to an antigen on the tumor cell surface and the other arm binds to an antigen on a T cell, an antibody drug conjugate, a bispecific antibody conjugated with an anti-tumor agent, a checkpoint inhibitor, for example, one that targets, PD-1 or CTLA-4, or combinations thereof. In certain embodiments, the checkpoint inhibitors may be selected from PD-1 inhibitors, such as pembrolizumab (Keytruda), nivolumab (Opdivo), or cemiplimab (REGN2810). In certain embodiments, the checkpoint inhibitors may be selected from PD-L1 inhibitors, such as atezolizumab (Tecentriq), avelumab (Bavencio), or Durvalumab (Imfinzi)). In certain embodiments, the checkpoint inhibitors may be selected from CTLA-4 inhibitors, such as ipilimumab (Yervoy). Other combinations that may be used in conjunction with an antibody of the disclosure are described above.

The present disclosure also includes therapeutic combinations comprising any of the antigen-binding molecules mentioned herein and an inhibitor of one or more of VEGF, Ang2, DLL4, EGFR, ErbB2, ErbB3, ErbB4, EGFRvIII, cMet, IGF1R, IL-10, B-raf, PDGFR-α, PDGFR-β, FOLH1 (PSMA), PRLR, STEAP1, STEAP2, TMPRSS2, MSLN, CA9, uroplakin, or any of the aforementioned cytokines, wherein the inhibitor is an aptamer, an antisense molecule, a ribozyme, an siRNA, a peptibody, a nanobody, an antibody, a bispecific antibody or an antibody fragment (e.g., Fab fragment; F(ab′)₂ fragment; Fd fragment; Fv fragment; scFv; dAb fragment; or other engineered molecules, such as diabodies, triabodies, tetrabodies, minibodies and minimal recognition units). The antigen-binding molecules of the disclosure may also be administered and/or co-formulated in combination with antivirals, antibiotics, analgesics, corticosteroids and/or NSAIDs. The antigen-binding molecules of the disclosure may also be administered as part of a treatment regimen that also includes radiation treatment and/or conventional chemotherapy.

The additional therapeutically active component(s) may be administered just prior to, concurrent with, or shortly after the administration of an antigen-binding molecule of the present disclosure; (for purposes of the present disclosure, such administration regimens are considered the administration of an antigen-binding molecule “in combination with” an additional therapeutically active component).

The present disclosure includes pharmaceutical compositions in which an antigen-binding molecule of the present disclosure is co-formulated with one or more of the additional therapeutically active component(s) as described elsewhere herein.

### EXAMPLES

The following examples are put forth so as to provide those of ordinary skill in the art with a complete disclosure and description of how to make and use the methods and compositions of the disclosure, and are not intended to limit the scope of what the inventors regard as their disclosure. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Protease Cleavage Validation for Masked Construct

Cleavability of the cleavable linkers (CLV) in the masked molecules of the present disclosure (e.g., the molecule of FIG. 1A) was confirmed in vitro using a recombinant human matriptase.

Proteins were diluted to 40 μg/ml in Dulbecco's PBS without Ca or Mg. Recombinant human matriptase (R&D Systems, 3946-SEB-010) was added at 1/100 (4.4 μg/ml final) and the proteins were incubated overnight at 37° C. with shaking. The following morning 15 μl was mixed with 4× Laemmli Sample Buffer (Bio-Rad Laboratories, 1610747) and dithiothreitol (Acros Organics, AC165680050, 1 mM final) and incubated at 95° C. for 5 minutes. The proteins were analyzed by SDS-PAGE and transferred to PVDF membranes (Invitrogen IB24001). The blots were blocked with 3% BSA in TBST and stained with a 1/5000 dilution of Peroxidase AffiniPure Goat Anti-Human IgG (H+L) (Jackson, 109-035-088). After washing with TBST, the blots were imaged with SuperSignal West Pico PLUS Chemiluminescent Substrate (ThermoFisher, 34577). Size was determined using the MagicMark XP Western Standard, ThermoFisher, LC5602).

The Western blots confirmed matriptase cleavage of the cleavable linker linking the pair of stacked Fabs and the cleavable linker linking the C-terminal Fab to the hinge and Fc domain, as illustrated in FIG. 1A.

### Example 2: Binding Validation for Masked Constructs

Binding of the masked and cleaved versions of the molecules (e.g., FIGS. 1A and 1B, respectively; and FIGS. 4D and 4H, respectively; and FIGS. 4E and 4I, respectively) of the present disclosure to CD3-expressing Jurkat cells and target antigen expressing cells was evaluated in vitro. In the molecules having the structures of FIGS. 4D, 4E, 4H and 4I, the CD3 binding domain was proximal to the Fc domain.

A serial titration of antibodies (pre-incubated with or without 4.4 ug/ml of recombinant Matriptase (R&D Systems, Cat #3946-SEB-010) were incubated with Jurkat cells or target antigen expressing cells for 30 min at 4° C. After wash, cells were incubated with a secondary antibody (anti-H+L APC, Cat #709-606-149, Jackson ImmunoResearch) for 30 min at 4° C. After being washed, cells were analyzed by Flow Cytometry on a FACS BD Celesta Flow Cytometer.

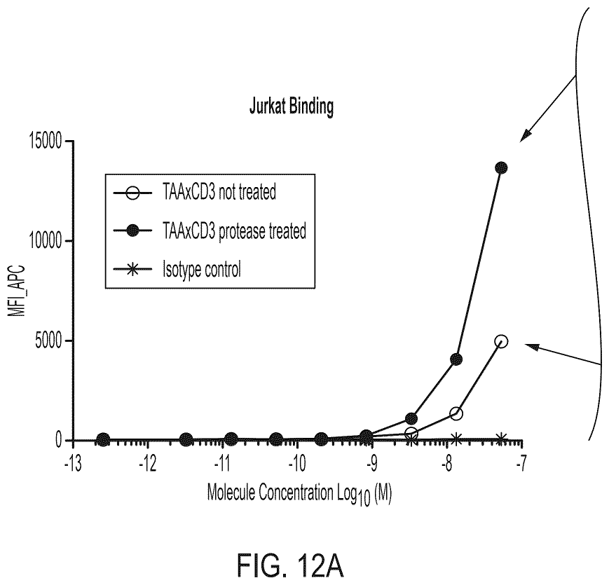

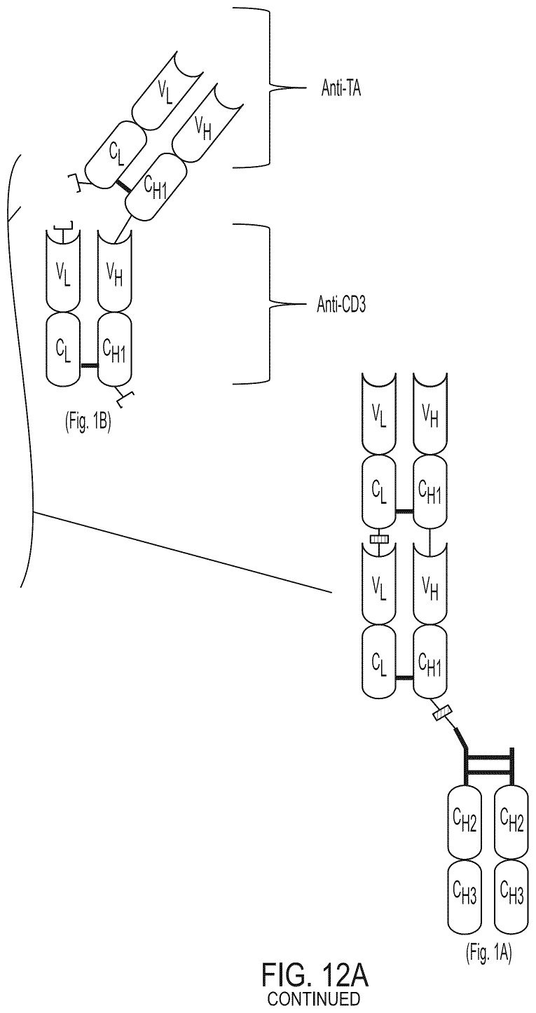

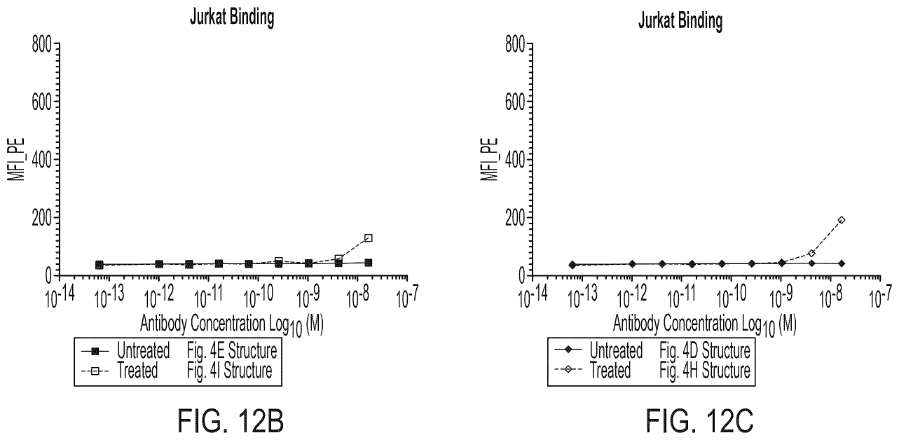

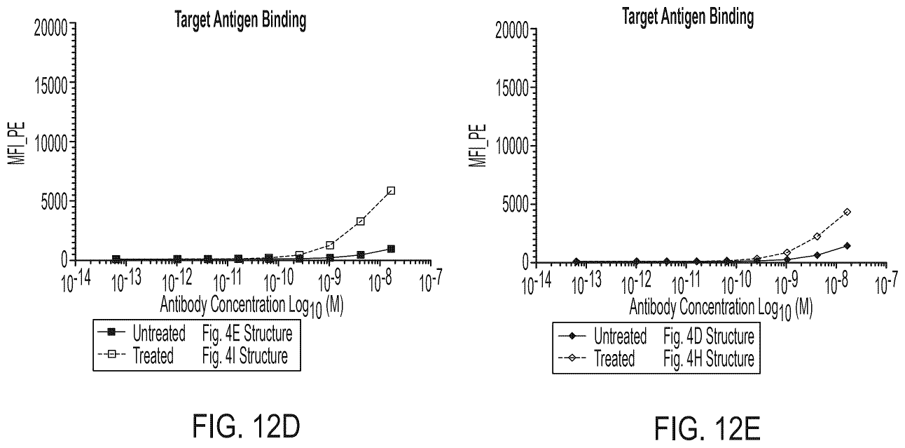

As shown in FIG. 12A, the protease treated (cleaved) version of the molecule (e.g., FIG. 1B) was demonstrated to have increased binding to the CD3-expressing Jurkat cells following treatment of the masked version of the molecule (e.g., FIG. 1A) with matriptase. Similarly, the protease treated (cleaved) version of the molecule (e.g., FIGS. 4I and 4H) was demonstrated to have increased binding to the CD3-expressing Jurkat cells (FIGS. 12B and 12C, respectively), and to target antigen-expressing cells (FIGS. 12D and 12E, respectively) following treatment of the masked versions of the molecules (e.g., FIGS. 4E and 4D, respectively).

### Example 3: Cytotoxicity and T-Cell Activation Validation for Masked Constructs

Cytotoxic activity of the masked and cleaved versions of the molecules (e.g., FIGS. 1A and 1B, respectively; and FIGS. 4E and 4I, respectively) of the present disclosure was evaluated in vitro. In the molecules having the structures of FIGS. 4E and 4I, the CD3 binding domain was proximal to the Fc domain

Molecules were incubated with or without 4.4 ug/ml Recombinant Matriptase (R&D systems, cat #3946-SEB-010) overnight at 37 C. At the same time, human PBMC were thawed and plated overnight for T cell enrichment. C4-2, 22RV1, and LNCaP cells (TAA+ tumor cell lines) were labeled with cell trace violet (Cat #34557, Invitrogen) and plated overnight. Next day, a serial titration of molecules treated+/−recombinant Matriptase were incubated with TAA+ tumor cells and adherent cell depleted hPBMC at a 4:1 E:T ratio for 48 h at 37° C. After incubation, cells were removed from cell culture plates using Trypsin-EDTA dissociation buffer, and analyzed by Flow cytometry on a FACS BD Celesta. Cells were stained with a cocktail of directly conjugated CD2, CD4, CD8, CD25 antibodies and live/dead near IR. For the assessment of cytotoxicity, cells were gated on live violet labeled populations. Percent of live population was recorded and used for the calculation of survival. T cell activation was assessed by reporting the percent of CD25+ T cells out of CD2+ T cells.

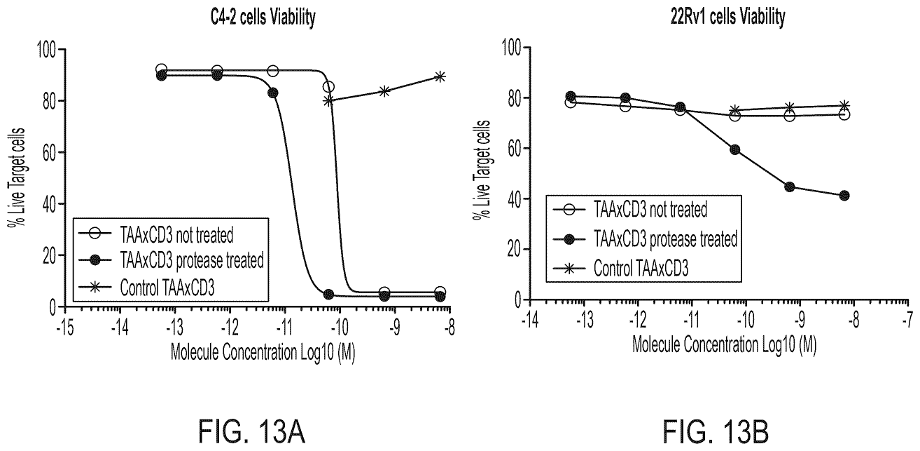

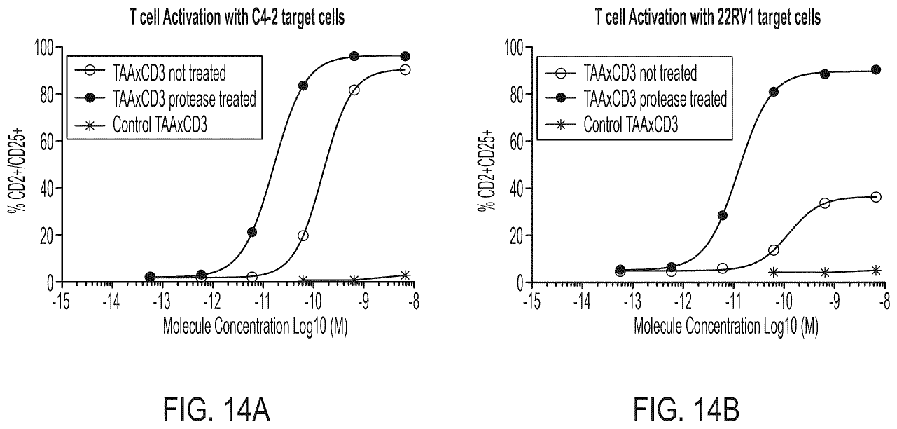

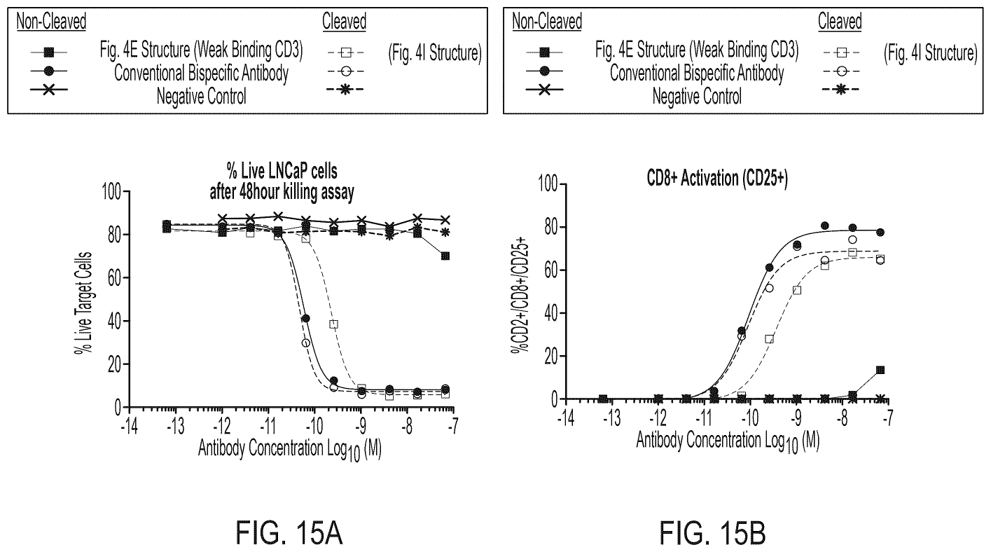

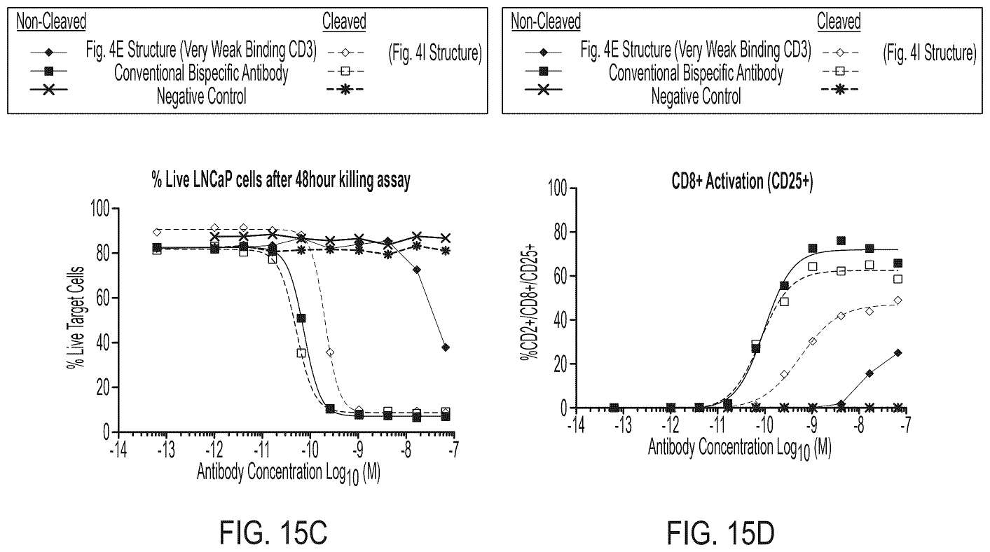

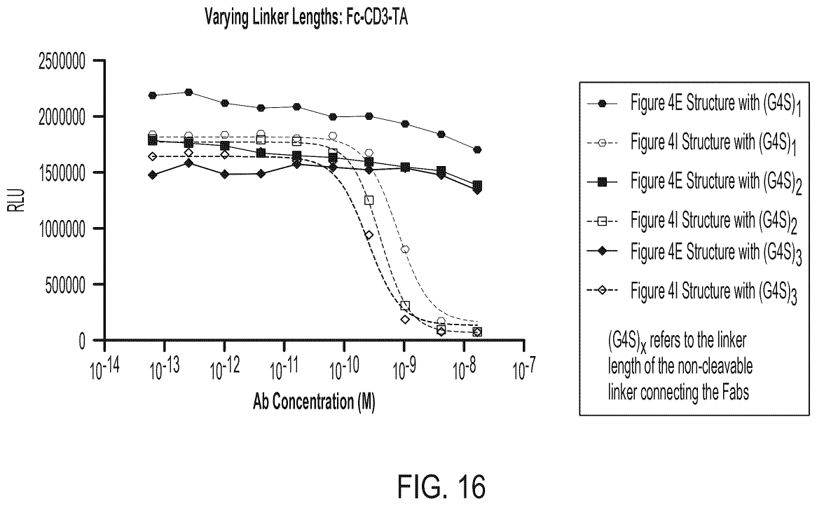

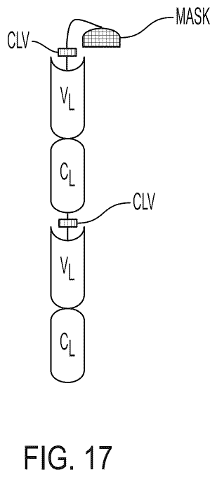

As shown in FIGS. 13A and 13B, the protease treated (cleaved) version of the molecule (e.g., FIG. 1B) was demonstrated to have increased cytotoxic potency following treatment of the masked version of the molecule (e.g., FIG. 1A) with matriptase. Similarly, as shown in FIGS. 14A and 14B, the protease treated (cleaved) version of the molecule (e.g., FIG. 1B) was demonstrated to have increased T-cell activation and increased potency for activation of T cells following treatment of the masked version of the molecule (e.g., FIG. 1A) with matriptase. Similar to the results discussed above, the protease treated (cleaved) version of the molecule (e.g., FIG. 4I) was demonstrated to have increased cytotoxic potency following treatment of the masked version of the molecule (e.g., FIG. 4E) with matriptase, as shown in FIGS. 15A and 15C, and the protease treated (cleaved) version of the molecule (e.g., FIG. 4I) was demonstrated to have increased T-cell activation and increased potency for activation of T cells following treatment of the masked version of the molecule (e.g., FIG. 4E) with matriptase, as shown in FIGS. 15B and 15D. The molecules of FIGS. 15A and 15B include an anti-CD3 binding domain that binds weakly (to CD3, whereas the molecules of FIGS. 15C and 15D include an anti-CD3 binding domain that binds very weakly to CD3.

### Example 4: Cytotoxicity of Various Masked Constructs

Cytotoxic activity of the masked and cleaved versions of the molecules shown in Table 10, below, was evaluated in vitro to determine the relative activity. The experiments were performed as discussed in Example 3, but with LNCaP cells (TAA+ tumor cell line) containing a Nanoluc® luciferase reporter. The TAA target was distinct from that of the molecules used in Example 3, and all molecules used the “G20” anti-CD3 binding domains. After incubation, cytotoxicity was assessed using NanoGlo™ kit and Envision™ for data acquisition. The assays were run in duplicate, and the results (fold change in cytotoxic activity from non-cleaved to cleaved molecules) are shown in Table 10.

TABLE 10
Fold Change in Cytotoxic Activity from Non-Cleaved to Cleaved

| | Assay 1 | Assay 2 | Average Fold |
|---|---|---|---|
| Molecule | (Fold Change) | (Fold Change) | Change |
| FIG. 1A, but with non-cleavable linkers between | 10.3 | 15.2 | 12.8 |
| the Fabs (control) | | | |
| FIG. 1A (CLV between light chains) | 4.9 | 7.3 | 6.1 |
| FIG. 1A, but with the CLV between the heavy | 3.2 | 5.5 | 4.4 |
| chains of the two Fabs | | | |
| FIG. 4A, but with non-cleavable linkers between | 3.8 | 13.2 | 8.5 |
| the Fabs (control), and with the N-terminal Fab | | | |
| targeting CD3, and the C-terminal Fab targeting | | | |
| TA | | | |
| FIG. 4A, with the N-terminal Fab targeting CD3, | 5.0 | 4.9 | 5.0 |
| and the C-terminal Fab targeting TA | | | |
| FIG. 4B, with the N-terminal Fab targeting CD3, | 33.0 | 33.5 | 33.3 |
| and the C-terminal Fab targeting TA | | | |
| FIG. 4C, but with non-cleavable linkers between | 8.1 | 6.4 | 7.3 |
| the Fabs on both sides(control), and with the N- | | | |
| terminal Fabs targeting CD3, and the C-terminal | | | |
| Fabs targeting TA | | | |
| FIG. 4C, with the N-terminal Fabs targeting CD3, | 9.5 | 4.5 | 7.0 |
| and the C-terminal Fabs targeting TA | | | |
| FIG. 4C, but with the CLV between the heavy | 2.4 | ND | 2.4 |
| chains of the two Fabs on both sides, and with the | | | |
| N-terminal Fabs targeting CD3, and the C- | | | |
| terminal Fabs targeting TA | | | |
| FIG. 4D, but with non-cleavable linkers between | 26.9 | 29.2 | 28.0 |
| the Fabs (control), and with the N-terminal Fab | | | |
| targeting CD3, and the C-terminal Fab targeting | | | |
| TA | | | |
| FIG. 4D, with the N-terminal Fab targeting CD3, | 18.5 | 18.0 | 18.3 |
| and the C-terminal Fab targeting TA | | | |
| FIG. 4E, with the N-terminal Fab targeting CD3, | 117.6 | 565.5 | 341.6 |
| and the C-terminal Fab targeting TA | | | |

As shown in Table 10, the construct having the structure of FIG. 4E, in which the cleavable linker separating the Fabs lies between the heavy chain portions of the two Fabs, showed a significant fold change in cytotoxic activity between the cleaved (following protease exposure) and non-cleaved versions of the molecule, particularly when compared against the fold change in activity for the other tested molecules, each of which contains the same antigen-binding domains. The construct having the structure of FIG. 4B, in which the cleavable linker separating the Fabs lies between the heavy chain portions of the two Fabs, also showed a significant, albeit less so, fold change in cytotoxic activity between the cleaved (following protease exposure) and non-cleaved versions of the molecule. Experiments also demonstrated that the presence of the CD3 binding domain proximal to the Fc region increased the fold change for cytotoxic activity between the cleaved (following protease exposure) and non-cleaved versions of the molecules having the structure of FIG. 4E/4I, relative to the same structure in which the CD3 binding domain was located distal to the Fc region (data not shown).

### Example 5: Effect of Non-Cleavable Linker Length on Cytotoxic Potency

Cytotoxic potency of the masked and cleaved versions of the molecules having the structures of FIGS. 4E and 4I, respectively, were evaluated with varying length non-cleavable linkers separating the tandem Fabs. Molecules were incubated with or without 4 ug/ml Recombinant Matriptase (R&D systems, cat #3946-SEB-010) overnight at 37 C. At the same time, human PBMC were thawed and plated overnight for T cell enrichment. LNCaP cells (TAA+ tumor cell line) containing a Nanoluc® luciferase reporter were plated overnight. Next day, a serial titration of molecules treated+/−recombinant Matriptase were incubated with TAA+ tumor cells and adherent cell depleted hPBMC at a 4:1 E:T ratio for 48 h at 37° C. After incubation, cytotoxicity was assessed using NanoGlo™ kit and Envision™ for data acquisition. As shown in FIG. 16, increasing the length of the non-cleavable linker connecting the tandom Fabs increased cytotoxic potency.

The present disclosure is not to be limited in scope by the specific embodiments described herein. Indeed, various modifications of the disclosure in addition to those described herein will become apparent to those skilled in the art from the foregoing description. Such modifications are intended to fall within the scope of the appended claims.

1. A multispecific antigen-binding molecule, comprising:
(a) a first immunoglobulin antigen-binding fragment (Fab) comprising a first polypeptide comprising, from N-terminus to C-terminus, a light chain variable region (LCVR) and an immunoglobulin light chain constant region (CL), and a second polypeptide comprising, from N-terminus to C-terminus, a heavy chain variable region (HCVR) and an immunoglobulin CH1 heavy chain constant region (CH1), wherein the first polypeptide and the second polypeptide are connected by a disulfide bond;
(b) a second immunoglobulin Fab comprising a first polypeptide comprising, from N-terminus to C-terminus, a LCVR and a CL, and a second polypeptide comprising, from N-terminus to C-terminus, a HCVR and a CH1; and
(c) an immunoglobulin Fc region comprising a pair of polypeptides, each comprising a hinge region, an immunoglobulin CH2 heavy chain constant region (CH2) and an immunoglobulin CH3 heavy chain constant region (CH3), wherein the pair of polypeptides is connected by a disulfide bond or a non-cleavable linker, optionally wherein the pair of polypeptides is connected by a disulfide bond in the hinge region,
wherein the first immunoglobulin Fab and the second immunoglobulin Fab are connected by a first linker between the C-terminus of the CL of the first immunoglobulin Fab and the N-terminus of the LCVR of the second immunoglobulin Fab, and by a second linker between the C-terminus of the CH1 of the first immunoglobulin Fab and the N-terminus of the HCVR of the second immunoglobulin Fab, and wherein the first linker or the second linker, but not both, is a protease cleavable linker, and
wherein the first immunoglobulin Fab or the second immunoglobulin Fab, but not both, is connected to the immunoglobulin Fc region by a third linker that is a protease cleavable linker.

2-22. (canceled)

23. The multispecific antigen-binding molecule of claim 1, wherein the first immunoglobulin Fab or the second immunoglobulin Fab binds a T-cell antigen or CD3.

24. The multispecific antigen-binding molecule of claim 1, wherein the first immunoglobulin Fab or the second immunoglobulin Fab binds a target antigen or a tumor-associated antigen.

25. The multispecific antigen-binding molecule of claim 1, further comprising:
(c) a third immunoglobulin Fab comprising a first polypeptide comprising, from N-terminus to C-terminus, a LCVR and a CL, and a second polypeptide comprising, from N-terminus to C-terminus, a HCVR and a CH1; and
(d) a fourth immunoglobulin Fab comprising a first polypeptide comprising, from N-terminus to C-terminus, a LCVR and a CL, and a second polypeptide comprising, from N-terminus to C-terminus, a HCVR and a CH1,
wherein the third immunoglobulin Fab and the fourth immunoglobulin Fab are connected by a fifth linker between the C-terminus of the CL of the third immunoglobulin Fab and the N-terminus of the LCVR of the fourth immunoglobulin Fab, and by a sixth linker between the C-terminus of the CH1 of the third immunoglobulin Fab and the N-terminus of the HCVR of the fourth immunoglobulin Fab, and wherein the fifth linker or the sixth linker, but not both, is a protease cleavable linker, and
wherein the third immunoglobulin Fab or the fourth immunoglobulin Fab, but not both, is connected to the immunoglobulin Fc region by a seventh linker that is a protease cleavable linker, and wherein (i) if the first immunoglobulin Fab is connected to the immunoglobulin Fc region, then the third immunoglobulin Fab is also connected to the immunoglobulin Fc region, or (ii) if the second immunoglobulin Fab is connected to the immunoglobulin Fc region, then the fourth immunoglobulin Fab is also connected to the immunoglobulin Fc region.

26-35. (canceled)

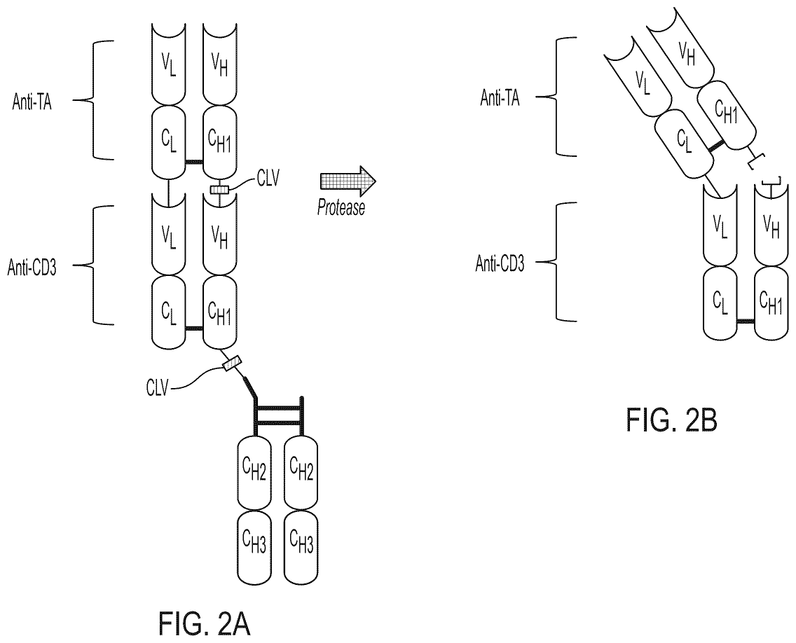

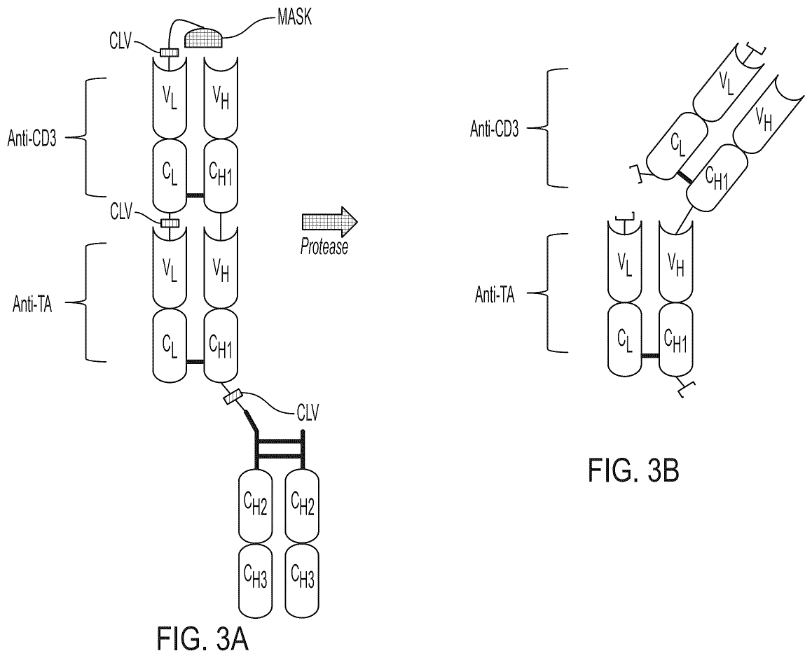

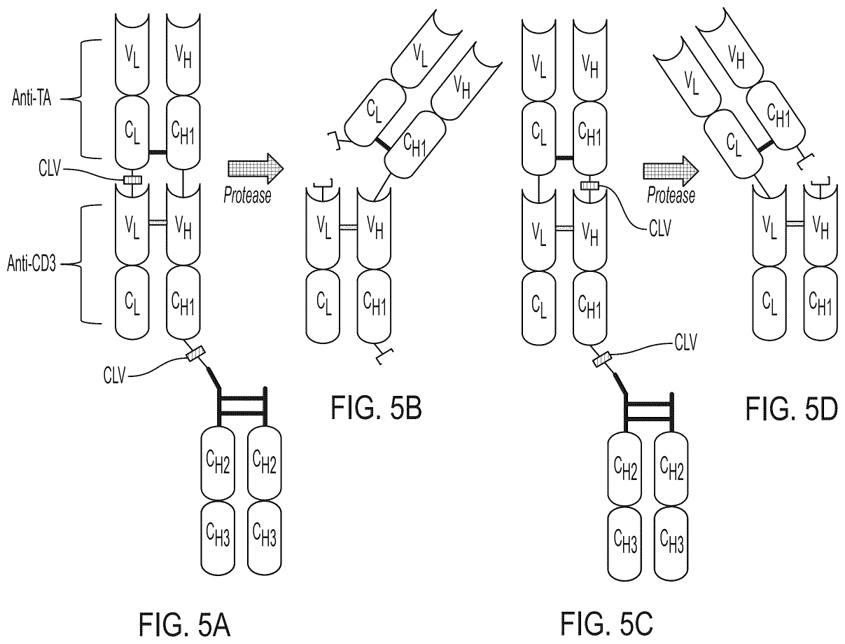

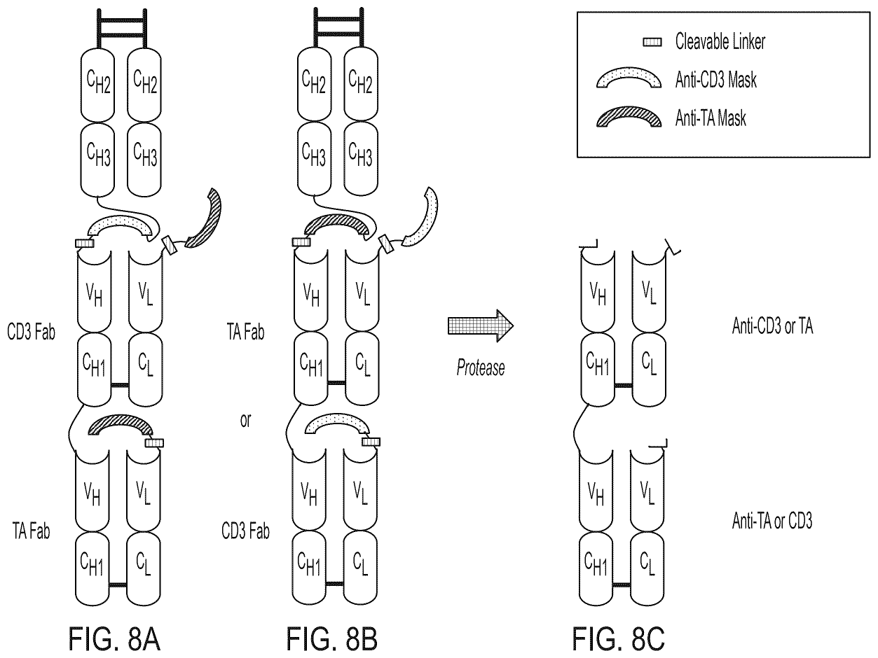

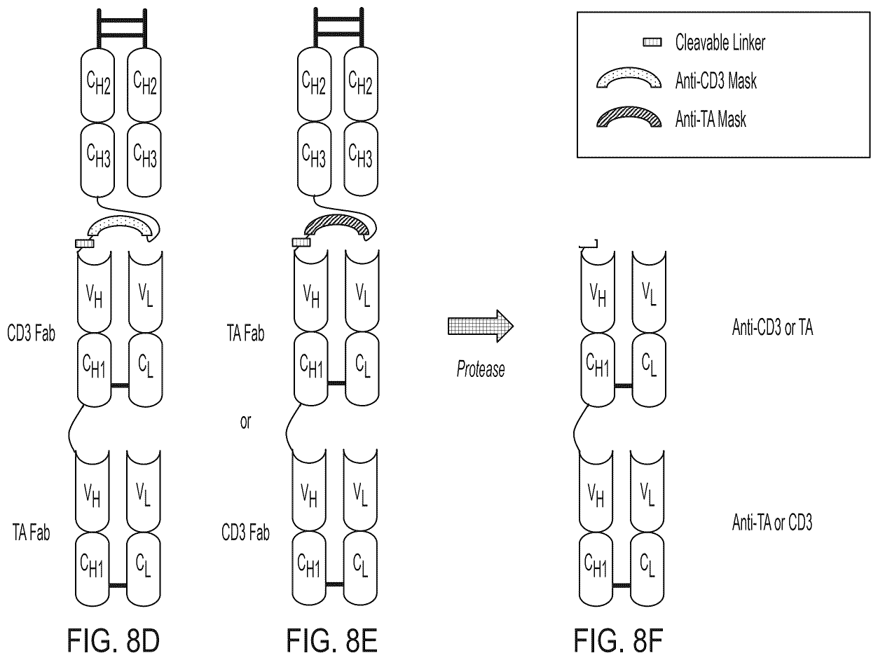

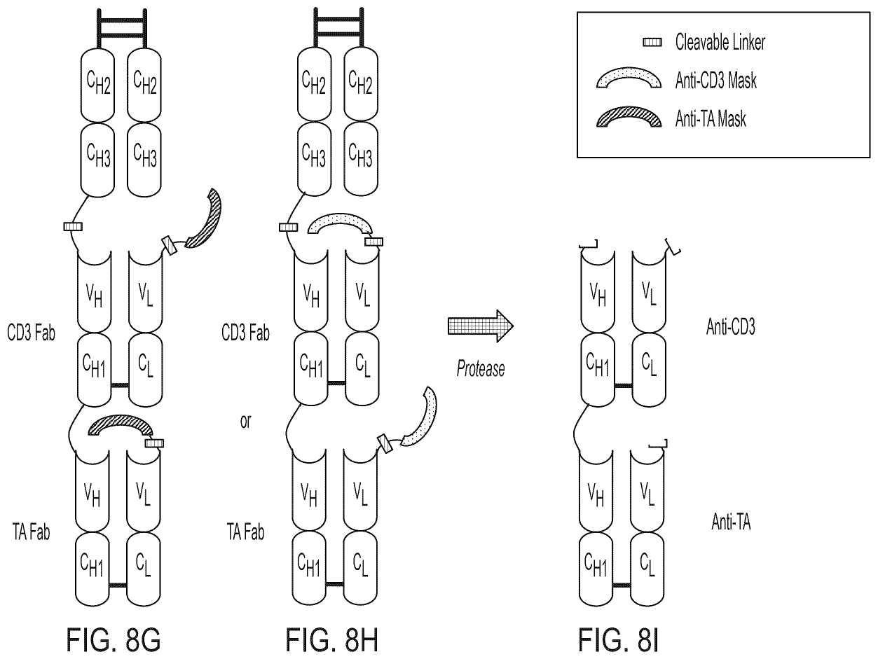

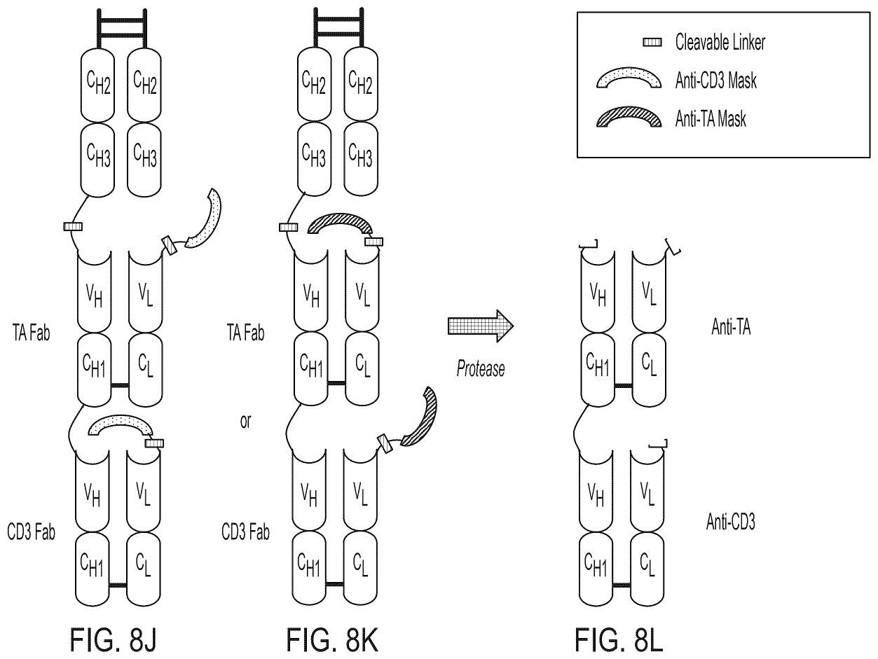

36. A multispecific antigen-binding molecule, comprising:
(A)
(a) a first immunoglobulin antigen-binding fragment (Fab) comprising a first polypeptide comprising, from N-terminus to C-terminus, a light chain variable region (LCVR) and an immunoglobulin light chain constant region (CL), and a second polypeptide comprising, from N-terminus to C-terminus, a heavy chain variable region (HCVR) and an immunoglobulin CH1 heavy chain constant region (CH1), wherein the first polypeptide and the second polypeptide are connected by a disulfide bond;
(b) a second immunoglobulin Fab comprising a first polypeptide comprising, from N-terminus to C-terminus, a LCVR and a CL, and a second polypeptide comprising, from N-terminus to C-terminus, a HCVR and a CH1; and
(c) an immunoglobulin Fc region comprising a first polypeptide and a second polypeptide, each polypeptide comprising an immunoglobulin CH2 heavy chain constant region (CH2) and an immunoglobulin CH3 heavy chain constant region (CH3), wherein the first and second polypeptides are connected by a disulfide bond or a non-cleavable linker,
wherein the first immunoglobulin Fab and the second immunoglobulin Fab are connected by a first linker between the C-terminus of the CL of the first immunoglobulin Fab and the N-terminus of the LCVR of the second immunoglobulin Fab, and by a second linker between the C-terminus of the CH1 of the first immunoglobulin Fab and the N-terminus of the HCVR of the second immunoglobulin Fab, and wherein the first linker is a non-cleavable linker and the second linker is a protease cleavable linker, and
wherein the first immunoglobulin Fab is connected to the immunoglobulin Fc region by a third linker that is a protease cleavable linker between the N-terminus of the LCVR of the first immunoglobulin Fab and the C-terminus of the CH3 of the first polypeptide of the immunoglobulin Fc region, and by a fourth linker that is a protease cleavable linker between the N-terminus of the HCVR of the first immunoglobulin Fab and the C-terminus of the CH3 of the second polypeptide of the immunoglobulin Fc region, or
(B)
(a) a first immunoglobulin antigen-binding fragment (Fab) comprising: a first polypeptide comprising, from N-terminus to C-terminus, a light chain variable region (LCVR) and an immunoglobulin light chain constant region (CL); and a second polypeptide comprising, from N-terminus to C-terminus, a peptide mask, a protease cleavable linker, a heavy chain variable region (HCVR) and an immunoglobulin CH1 heavy chain constant region (CH1), wherein the first polypeptide and the second polypeptide are connected by a disulfide bond;
(b) a second immunoglobulin Fab comprising: a first polypeptide comprising, from N-terminus to C-terminus, a LCVR and a CL; and a second polypeptide comprising, from N-terminus to C-terminus, a HCVR and a CH1, wherein the first polypeptide and the second polypeptide are connected by a disulfide bond; and
(c) an immunoglobulin Fc region comprising a pair of polypeptides, each comprising a hinge region, an immunoglobulin CH2 heavy chain constant region (CH2) and an immunoglobulin CH3 heavy chain constant region (CH3), wherein the pair of polypeptides are connected by a disulfide bond or a non-cleavable linker,
wherein the first immunoglobulin Fab and the second immunoglobulin Fab are connected by a non-cleavable linker between the C-terminus of the CH1 of the first immunoglobulin Fab and the N-terminus of the HCVR of the second immunoglobulin Fab, and
wherein the first immunoglobulin Fab is connected to the immunoglobulin Fc region by a non-cleavable linker between the peptide mask of the second polypeptide and a CH3 of the immunoglobulin Fc region, or
(C)
(a) a first immunoglobulin antigen-binding fragment (Fab) comprising: a first polypeptide comprising, from N-terminus to C-terminus, a peptide mask, a protease cleavable linker, a light chain variable region (LCVR) and an immunoglobulin light chain constant region (CL); and a second polypeptide comprising, from N-terminus to C-terminus, a heavy chain variable region (HCVR) and an immunoglobulin CH1 heavy chain constant region (CH1), wherein the first polypeptide and the second polypeptide are connected by a disulfide bond;
(b) a second immunoglobulin Fab comprising: a first polypeptide comprising, from N-terminus to C-terminus, a peptide mask, a protease cleavable linker, a LCVR and a CL; and a second polypeptide comprising, from N-terminus to C-terminus, a HCVR and a CH1, wherein the first polypeptide and the second polypeptide are connected by a disulfide bond; and
(c) an immunoglobulin Fc region comprising a pair of polypeptides, each comprising a hinge region, an immunoglobulin CH2 heavy chain constant region (CH2) and an immunoglobulin CH3 heavy chain constant region (CH3), wherein the pair of polypeptides are connected by a disulfide bond or a non-cleavable linker,
wherein the first immunoglobulin Fab and the second immunoglobulin Fab are connected by a non-cleavable linker between the C-terminus of the CH1 of the first immunoglobulin Fab and the N-terminus of the HCVR of the second immunoglobulin Fab, and
wherein the first immunoglobulin Fab is connected to the immunoglobulin Fc region by a protease cleavable linker between the HCVR of the second polypeptide and a CH3 of the immunoglobulin Fc region, or
(D)
(a) a first immunoglobulin antigen-binding fragment (Fab) comprising: a first polypeptide comprising, from N-terminus to C-terminus, a peptide mask, a protease cleavable linker, a light chain variable region (LCVR) and an immunoglobulin light chain constant region (CL); and a second polypeptide comprising, from N-terminus to C-terminus, a peptide mask, a protease cleavable linker, a heavy chain variable region (HCVR) and an immunoglobulin CH1 heavy chain constant region (CH1), wherein the first polypeptide and the second polypeptide are connected by a disulfide bond;
(b) a second immunoglobulin Fab comprising: a first polypeptide comprising, from N-terminus to C-terminus, a peptide mask, a protease cleavable linker, a LCVR and a CL; and a second polypeptide comprising, from N-terminus to C-terminus, a HCVR and a CH1, wherein the first polypeptide and the second polypeptide are connected by a disulfide bond;
(c) a third immunoglobulin Fab comprising: a first polypeptide comprising, from N-terminus to C-terminus, a peptide mask, a protease cleavable linker, a LCVR and a CL; and a second polypeptide comprising, from N-terminus to C-terminus, a peptide mask, a protease cleavable linker, a HCVR and a CH1, wherein the first polypeptide and the second polypeptide are connected by a disulfide bond;
(d) a fourth immunoglobulin Fab comprising: a first polypeptide comprising, from N-terminus to C-terminus, a peptide mask, a protease cleavable linker, a LCVR and a CL; and a second polypeptide comprising, from N-terminus to C-terminus, a HCVR and a CH1, wherein the first polypeptide and the second polypeptide are connected by a disulfide bond; and
(e) an immunoglobulin Fc region comprising a pair of polypeptides, each comprising a hinge region, an immunoglobulin CH2 heavy chain constant region (CH2) and an immunoglobulin CH3 heavy chain constant region (CH3), wherein the pair of polypeptides are connected by a disulfide bond or a non-cleavable linker,
wherein the first immunoglobulin Fab and the second immunoglobulin Fab are connected by a non-cleavable linker between the C-terminus of the CH1 of the first immunoglobulin Fab and the N-terminus of the HCVR of the second immunoglobulin Fab,
wherein the third immunoglobulin Fab and the fourth immunoglobulin Fab are connected by a non-cleavable linker between the C-terminus of the CH1 of the third immunoglobulin Fab and the N-terminus of the HCVR of the fourth immunoglobulin Fab,
wherein the first immunoglobulin Fab is connected to the immunoglobulin Fc region by a non-cleavable linker between the peptide mask of the second polypeptide and a CH3 of the immunoglobulin Fc region, and
wherein the third immunoglobulin Fab is connected to the immunoglobulin Fc region by a non-cleavable linker between the peptide mask of the second polypeptide and a CH3 of the immunoglobulin Fc region, or
(E)
(a) a first immunoglobulin antigen-binding fragment (Fab) comprising: a first polypeptide comprising, from N-terminus to C-terminus, a light chain variable region (LCVR) and an immunoglobulin light chain constant region (CL); and a second polypeptide comprising, from N-terminus to C-terminus, a peptide mask, a protease cleavable linker, a heavy chain variable region (HCVR) and an immunoglobulin CH1 heavy chain constant region (CH1), wherein the first polypeptide and the second polypeptide are connected by a disulfide bond;
(b) a second immunoglobulin Fab comprising: a first polypeptide comprising, from N-terminus to C-terminus, a LCVR and a CL; and a second polypeptide comprising, from N-terminus to C-terminus, a HCVR and a CH1, wherein the first polypeptide and the second polypeptide are connected by a disulfide bond;
(c) a third immunoglobulin Fab comprising: a first polypeptide comprising, from N-terminus to C-terminus, a LCVR and a CL; and a second polypeptide comprising, from N-terminus to C-terminus, a peptide mask, a protease cleavable linker, a HCVR and a CH1, wherein the first polypeptide and the second polypeptide are connected by a disulfide bond;
(d) a fourth immunoglobulin Fab comprising: a first polypeptide comprising, from N-terminus to C-terminus, a LCVR and a CL; and a second polypeptide comprising, from N-terminus to C-terminus, a HCVR and a CH1, wherein the first polypeptide and the second polypeptide are connected by a disulfide bond; and
(e) an immunoglobulin Fc region comprising a pair of polypeptides, each comprising a hinge region, an immunoglobulin CH2 heavy chain constant region (CH2) and an immunoglobulin CH3 heavy chain constant region (CH3), wherein the pair of polypeptides are connected by a disulfide bond or a non-cleavable linker,
wherein the first immunoglobulin Fab and the second immunoglobulin Fab are connected by a non-cleavable linker between the C-terminus of the CH1 of the first immunoglobulin Fab and the N-terminus of the HCVR of the second immunoglobulin Fab,
wherein the third immunoglobulin Fab and the fourth immunoglobulin Fab are connected by a non-cleavable linker between the C-terminus of the CH1 of the third immunoglobulin Fab and the N-terminus of the HCVR of the fourth immunoglobulin Fab,
wherein the first immunoglobulin Fab is connected to the immunoglobulin Fc region by a non-cleavable linker between the peptide mask of the second polypeptide and a CH3 of the immunoglobulin Fc region, and
wherein the third immunoglobulin Fab is connected to the immunoglobulin Fc region by a non-cleavable linker between the peptide mask of the second polypeptide and a CH3 of the immunoglobulin Fc region, or
(F)
(a) a first immunoglobulin antigen-binding fragment (Fab) comprising: a first polypeptide comprising, from N-terminus to C-terminus, a peptide mask, a protease cleavable linker, a light chain variable region (LCVR) and an immunoglobulin light chain constant region (CL); and a second polypeptide comprising, from N-terminus to C-terminus, a heavy chain variable region (HCVR) and an immunoglobulin CH1 heavy chain constant region (CH1), wherein the first polypeptide and the second polypeptide are connected by a disulfide bond;
(b) a second immunoglobulin Fab comprising: a first polypeptide comprising, from N-terminus to C-terminus, a peptide mask, a protease cleavable linker, a LCVR and a CL; and a second polypeptide comprising, from N-terminus to C-terminus, a HCVR and a CH1, wherein the first polypeptide and the second polypeptide are connected by a disulfide bond;
(c) a third immunoglobulin Fab comprising: a first polypeptide comprising, from N-terminus to C-terminus, a peptide mask, a protease cleavable linker, a LCVR and a CL; and a second polypeptide comprising, from N-terminus to C-terminus, a HCVR and a CH1, wherein the first polypeptide and the second polypeptide are connected by a disulfide bond;
(d) a fourth immunoglobulin Fab comprising: a first polypeptide comprising, from N-terminus to C-terminus, a peptide mask, a protease cleavable linker, a LCVR and a CL; and a second polypeptide comprising, from N-terminus to C-terminus, a HCVR and a CH1, wherein the first polypeptide and the second polypeptide are connected by a disulfide bond; and
(e) an immunoglobulin Fc region comprising a pair of polypeptides, each comprising a hinge region, an immunoglobulin CH2 heavy chain constant region (CH2) and an immunoglobulin CH3 heavy chain constant region (CH3), wherein the pair of polypeptides are connected by a disulfide bond or a non-cleavable linker,
wherein the first immunoglobulin Fab and the second immunoglobulin Fab are connected by a non-cleavable linker between the C-terminus of the CH1 of the first immunoglobulin Fab and the N-terminus of the HCVR of the second immunoglobulin Fab,
wherein the third immunoglobulin Fab and the fourth immunoglobulin Fab are connected by a non-cleavable linker between the C-terminus of the CH1 of the third immunoglobulin Fab and the N-terminus of the HCVR of the fourth immunoglobulin Fab,
wherein the first immunoglobulin Fab is connected to the immunoglobulin Fc region by a protease cleavable linker between the HCVR of the second polypeptide and a CH3 of the immunoglobulin Fc region, and
wherein the third immunoglobulin Fab is connected to the immunoglobulin Fc region by a protease cleavable linker between the HCVR of the second polypeptide and a CH3 of the immunoglobulin Fc region, or
(G)
the structure of any one of FIGS. 1A, 1B, 1C, 1D, 1E, 1F, 1G, 2A, 2B, 3A, 3B, 4A, 4B, 4C, 4D, 4E, 4F, 4G, 4H, 4I, 5A, 5B, 5C, 5D, 6A, 6B, 7A, 7B, 8A, 8B, 8C, 8D, 8E, 8F, 8G, 8H, 8I, 8J, 8K, 8L, 9A, 9B, 9C, 10A, 10B, 10C, 10D, 11A, 11B, 11C and 11D.

37-45. (canceled)

46. A multispecific antigen-binding molecule, comprising:
(a) a first immunoglobulin antigen-binding fragment (Fab) comprising: a first polypeptide comprising, from N-terminus to C-terminus, a peptide mask, a protease cleavable linker, a light chain variable region (LCVR) and an immunoglobulin light chain constant region (CL); and a second polypeptide comprising, from N-terminus to C-terminus, a peptide mask, a protease cleavable linker, a heavy chain variable region (HCVR) and an immunoglobulin CH1 heavy chain constant region (CH1), wherein the first polypeptide and the second polypeptide are connected by a disulfide bond;
(b) a second immunoglobulin Fab comprising: a first polypeptide comprising, from N-terminus to C-terminus, a peptide mask, a protease cleavable linker, a LCVR and a CL; and a second polypeptide comprising, from N-terminus to C-terminus, a HCVR and a CH1, wherein the first polypeptide and the second polypeptide are connected by a disulfide bond; and
(c) an immunoglobulin Fc region comprising a pair of polypeptides, each comprising a hinge region, an immunoglobulin CH2 heavy chain constant region (CH2) and an immunoglobulin CH3 heavy chain constant region (CH3), wherein the pair of polypeptides are connected by a disulfide bond or a non-cleavable linker,
wherein the first immunoglobulin Fab and the second immunoglobulin Fab are connected by a non-cleavable linker between the C-terminus of the CH1 of the first immunoglobulin Fab and the N-terminus of the HCVR of the second immunoglobulin Fab, and
wherein the first immunoglobulin Fab is connected to the immunoglobulin Fc region by a non-cleavable linker between the peptide mask of the second polypeptide and a CH3 of the immunoglobulin Fc region.

47-48. (canceled)

49. The multispecific antigen-binding molecule of claim 46, wherein the immunoglobulin Fc region is an immunoglobulin Fc region of an antibody, wherein the antibody comprises two Fabs, each comprising a first polypeptide comprising, from N-terminus to C-terminus, a peptide mask, a protease cleavable linker, a LCVR and a CL, and a second polypeptide comprising a HCVR and a CH1, wherein the first and second polypeptide are connected by a disulfide bond, and the wherein the CH1 of each of the two Fabs is connected to the hinge region of the immunoglobulin Fc region.

50. The multispecific antigen-binding molecule of claim 46, wherein the first immunoglobulin Fab binds a T-cell antigen, and the second immunoglobulin Fab binds a target antigen.

51. The multispecific antigen-binding molecule of claim 46, wherein the first immunoglobulin Fab binds a target antigen, and the second immunoglobulin Fab binds a T-cell antigen.

52-59. (canceled)

60. The multispecific antigen-binding molecule of claim 46, wherein each polypeptide comprising a peptide mask, a protease cleavable linker, a LCVR and a CL, is identical.

61. The multispecific antigen-binding molecule of claim 46, wherein each peptide mask is an anti-idiotype antigen-binding molecule that binds either a target antigen binding domain of a Fab or a T-cell antigen binding domain of a Fab, but not both.

62. (canceled)

63. The multispecific antigen-binding molecule of claim 46, wherein the non-cleavable linker (a) consists of glycine and serine residues, or (b) is a linker selected from the linkers set forth in Table 9.

64. The multispecific antigen-binding molecule of claim 46, wherein the protease cleavable linker is a linker comprising from 2 to 100 amino acids and containing a substrate for a protease.

65. The multispecific antigen-binding molecule of claim 64, wherein the protease cleavable linker comprises from 2 to 50 amino acids, or from 5 to 50 amino acids, or from 2 to 25 amino acids, or from 5 to 25 amino acids, or from 2 to 20 amino acids, or from 5 to 20 amino acids, or from 2 to 15 amino acids, or from 5 to 15 amino acids, or from 2 to 10 amino acids, or from 5 to 10 amino acids.

66. (canceled)

67. The multispecific antigen-binding molecule of claim 46 that is bispecific.

68. A nucleic acid or plurality of nucleic acids encoding the multispecific antigen-binding molecule of claim 46.

69. An isolated cell transfected with one or more expression vectors comprising one or more nucleic acid sequences encoding the multispecific antigen-binding molecule of claim 46.

70. A method of producing the multispecific antigen-binding molecule of claim 46, comprising:
(a) culturing the cell of claim 69 in conditions under which the multispecific antigen-binding molecule is expressed; and
(b) recovering the multispecific antigen-binding molecule from the cell culture.

71. The method of claim 70, further comprising formulating the multispecific antigen-binding molecule with a pharmaceutically acceptable excipient or diluent, optionally wherein the multispecific antigen-binding molecule is purified prior to being formulated with the pharmaceutically acceptable excipient or diluent.

72. A pharmaceutical composition comprising the multispecific antigen-binding molecule of claim 46, and a pharmaceutically acceptable carrier or diluent.

73. A method of treating cancer, comprising administering the multispecific antigen-binding molecule of claim 46 to a subject in need thereof.

74. The method of claim 73, wherein the multispecific antigen-binding molecule is administered in combination with a second therapeutic agent.

75-86. (canceled)


SEQUENCE LISTING (485 sequences)

SEQ ID NO: 1 - DNA, 372 bp, synthetic construct
  misc_feature 1..372: note = synthetic
  source 1..372: mol_type = other DNA
gaagtacagcttgtagaatccggcggaggactggtacaacctggaagaagtcttagactgagttgcgcagctagtgggtttacattcgacgattacagcatgcattgggtgaggcaagctcctggtaaaggattggaatgggttagcgggatatcatggaactcaggaagcaagggatacgccgacagcgtgaaaggccgatttacaatatctagggacaacgcaaaaaactctctctaccttcaaatgaactctcttagggcagaagacacagcattgtattattgcgcaaaatacggcagtggttatggcaagttttatcattatggactggacgtgtggggacaagggacaacagtgacagtgagtagc

SEQ ID NO: 2 - protein, 124 aa, synthetic construct
  REGION 1..124: note = synthetic
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSS

SEQ ID NO: 3 - DNA, 24 bp, synthetic construct
  misc_feature 1..24: note = synthetic
  source 1..24: mol_type = other DNA
gggtttacattcgacgattacagc

SEQ ID NO: 4 - protein, 8 aa, synthetic construct
  REGION 1..8: note = synthetic
GFTFDDYS

SEQ ID NO: 5 - DNA, 24 bp, synthetic construct
  misc_feature 1..24: note = synthetic
  source 1..24: mol_type = other DNA
atatcatggaactcaggaagcaag

SEQ ID NO: 6 - protein, 8 aa, synthetic construct
  REGION 1..8: note = synthetic
ISWNSGSK

SEQ ID NO: 7 - DNA, 51 bp, synthetic construct
  misc_feature 1..51: note = synthetic
  source 1..51: mol_type = other DNA
gcaaaatacggcagtggttatggcaagttttatcattatggactggacgtg

SEQ ID NO: 8 - protein, 17 aa, synthetic construct
  REGION 1..17: note = synthetic
AKYGSGYGKFYHYGLDV

SEQ ID NO: 9 - DNA, 372 bp, synthetic construct
  misc_feature 1..372: note = synthetic
  source 1..372: mol_type = other DNA
gaagtacagttggtagaatctggaggaggactcgtgcaaccaggacgatcattgcggttgagttgtgctgctagtggattcacattcgacgactatgctatgcattgggtaagacaggctccaggaaaaggactcgaatgggtgtcaggaataagttggaactccggaagcattgggtacgcagattcagtcaaagggcgattcaccatatcccgagataacgctaagaactcactttaccttcaaatgaactctcttcgagcagaggacactgcactttattattgcgctaaggacggctccggttatggatatttttattattatggaatggacgtatggggacaaggcactactgttaccgttagttcc

SEQ ID NO: 10 - protein, 124 aa, synthetic construct
  REGION 1..124: note = synthetic
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDGSGYGYFYYYGMDVWGQGTTVTVSS

SEQ ID NO: 11 - DNA, 24 bp, synthetic construct
  misc_feature 1..24: note = synthetic
  source 1..24: mol_type = other DNA
ggattcacattcgacgactatgct

SEQ ID NO: 12 - protein, 8 aa, synthetic construct
  REGION 1..8: note = synthetic
GFTFDDYA

SEQ ID NO: 13 - DNA, 24 bp, synthetic construct
  misc_feature 1..24: note = synthetic
  source 1..24: mol_type = other DNA
ataagttggaactccggaagcatt

SEQ ID NO: 14 - protein, 8 aa, synthetic construct
  REGION 1..8: note = synthetic
ISWNSGSI

SEQ ID NO: 15 - DNA, 51 bp, synthetic construct
  misc_feature 1..51: note = synthetic
  source 1..51: mol_type = other DNA
gctaaggacggctccggttatggatatttttattattatggaatggacgta

SEQ ID NO: 16 - protein, 17 aa, synthetic construct
  REGION 1..17: note = synthetic
AKDGSGYGYFYYYGMDV

SEQ ID NO: 17 - DNA, 372 bp, synthetic construct
  misc_feature 1..372: note = synthetic
  source 1..372: mol_type = other DNA
gaagtacaactggtcgaatctggaggaggtcttgttcaacctggtcgatcacttcgcctttcttgtgccgcttctggtttcactttcgacgattatagcatgcattgggtacgacaggctcccggaaaagggctggaatgggtgtcaggaattagttggaactcaggaagtattggatacgctgattcagtcaaaggacgcttcacaatctcaagggacaacgctaaaaactcactttatttgcaaatgaactctctccgcgctgaagataccgctctctattattgcgccaaagatgggtctggttacggttatttttactactatggaatggacgtttggggccaaggaacaactgtcacagtatcatcc

SEQ ID NO: 18 - protein, 124 aa, synthetic construct
  REGION 1..124: note = synthetic
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKDGSGYGYFYYYGMDVWGQGTTVTVSS

SEQ ID NO: 19 - DNA, 24 bp, synthetic construct
  misc_feature 1..24: note = synthetic
  source 1..24: mol_type = other DNA
ggtttcactttcgacgattatagc

SEQ ID NO: 20 - protein, 8 aa, synthetic construct
  REGION 1..8: note = synthetic
GFTFDDYS

SEQ ID NO: 21 - DNA, 24 bp, synthetic construct
  misc_feature 1..24: note = synthetic
  source 1..24: mol_type = other DNA
attagttggaactcaggaagtatt

SEQ ID NO: 22 - protein, 8 aa, synthetic construct
  REGION 1..8: note = synthetic
ISWNSGSI

SEQ ID NO: 23 - DNA, 51 bp, synthetic construct
  misc_feature 1..51: note = synthetic
  source 1..51: mol_type = other DNA
gccaaagatgggtctggttacggttatttttactactatggaatggacgtt

SEQ ID NO: 24 - protein, 17 aa, synthetic construct
  REGION 1..17: note = synthetic
AKDGSGYGYFYYYGMDV

SEQ ID NO: 25 - DNA, 372 bp, synthetic construct
  misc_feature 1..372: note = synthetic
  source 1..372: mol_type = other DNA
gaagttcaacttgtggaaagtggcggaggattggttcaaccaggacgttcattgaggctttcatgcgcagcttccggatttacatttgacgattacgcaatgcactgggttagacaggcaccaggaaaaggactggagtgggtgagcgggatttcatggaacagcggcagtatcggttatgcagactcagttaaaggaagattcaccatcagtagagacaacgcaaaaaattccctttatctccaaatgaactctcttagggccgaagatacagcattgtactactgcgcaaaagacggatcaggttacggaaaattttactactatggtatggatgtatggggtcagggaaccacagtaactgtatcaagc

SEQ ID NO: 26 - protein, 124 aa, synthetic construct
  REGION 1..124: note = synthetic
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDGSGYGKFYYYGMDVWGQGTTVTVSS

SEQ ID NO: 27 - DNA, 24 bp, synthetic construct
  misc_feature 1..24: note = synthetic
  source 1..24: mol_type = other DNA
ggatttacatttgacgattacgca

SEQ ID NO: 28 - protein, 8 aa, synthetic construct
  REGION 1..8: note = synthetic
GFTFDDYA

SEQ ID NO: 29 - DNA, 24 bp, synthetic construct
  misc_feature 1..24: note = synthetic
  source 1..24: mol_type = other DNA
atttcatggaacagcggcagtatc

SEQ ID NO: 30 - protein, 8 aa, synthetic construct
  REGION 1..8: note = synthetic
ISWNSGSI

SEQ ID NO: 31 - DNA, 51 bp, synthetic construct
  misc_feature 1..51: note = synthetic
  source 1..51: mol_type = other DNA
gcaaaagacggatcaggttacggaaaattttactactatggtatggatgta

SEQ ID NO: 32 - protein, 17 aa, synthetic construct
  REGION 1..17: note = synthetic
AKDGSGYGKFYYYGMDV

SEQ ID NO: 33 - DNA, 372 bp, synthetic construct
  misc_feature 1..372: note = synthetic
  source 1..372: mol_type = other DNA
gaagtgcaactcgttgaaagcggaggaggactggtccagcccggcagatctctcagattgtcttgcgctgcatccggatttacatttgacgactattcaatgcactgggtacggcaagccccaggtaaaggactcgaatgggtaagcggcatatcttggaactcaggcagtattggctacgcagattcagtaaaaggaagattcactatttcaagggataatgctaagaacagtctctacttgcaaatgaatagcttgcgcgcagaagatacagcactttattattgtgcaaaagatggaagcggttatgggaaattttattattatggtatggatgtatggggtcaaggtacaacagttactgtgtcaagt

SEQ ID NO: 34 - protein, 124 aa, synthetic construct
  REGION 1..124: note = synthetic
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKDGSGYGKFYYYGMDVWGQGTTVTVSS

SEQ ID NO: 35 - DNA, 24 bp, synthetic construct
  misc_feature 1..24: note = synthetic
  source 1..24: mol_type = other DNA
ggatttacatttgacgactattca

SEQ ID NO: 36 - protein, 8 aa, synthetic construct
  REGION 1..8: note = synthetic
GFTFDDYS

SEQ ID NO: 37 - DNA, 24 bp, synthetic construct
  misc_feature 1..24: note = synthetic
  source 1..24: mol_type = other DNA
atatcttggaactcaggcagtatt

SEQ ID NO: 38 - protein, 8 aa, synthetic construct
  REGION 1..8: note = synthetic
ISWNSGSI

SEQ ID NO: 39 - DNA, 51 bp, synthetic construct
  misc_feature 1..51: note = synthetic
  source 1..51: mol_type = other DNA
gcaaaagatggaagcggttatgggaaattttattattatggtatggatgta

SEQ ID NO: 40 - protein, 17 aa, synthetic construct
  REGION 1..17: note = synthetic
AKDGSGYGKFYYYGMDV

SEQ ID NO: 41 - DNA, 372 bp, synthetic construct
  misc_feature 1..372: note = synthetic
  source 1..372: mol_type = other DNA
gaagtacagcttgtagaatccggcggaggactggtacaacctggaagaagtcttagactgagttgcgcagctagtgggtttacattcgacgattacagcatgcattgggtgaggcaagctcctggtaaaggattggaatgggttagcgggatatcatggaactcaggaagcatcggatacgccgacagcgtgaaaggccgatttacaatatctagggacaacgcaaaaaactctctctaccttcaaatgaactctcttagggcagaagacacagcattgtattattgcgcaaaatacggcagtggttatggcaagttttatcattatggactggacgtgtggggacaagggacaacagtgacagtgagtagc

SEQ ID NO: 42 - protein, 124 aa, synthetic construct
  REGION 1..124: note = synthetic
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYHYGLDVWGQGTTVTVSS

SEQ ID NO: 43 - DNA, 24 bp, synthetic construct
  misc_feature 1..24: note = synthetic
  source 1..24: mol_type = other DNA
gggtttacattcgacgattacagc

SEQ ID NO: 44 - protein, 8 aa, synthetic construct
  REGION 1..8: note = synthetic
GFTFDDYS

SEQ ID NO: 45 - DNA, 24 bp, synthetic construct
  misc_feature 1..24: note = synthetic
  source 1..24: mol_type = other DNA
atatcatggaactcaggaagcatc

SEQ ID NO: 46 - protein, 8 aa, synthetic construct
  REGION 1..8: note = synthetic
ISWNSGSI

SEQ ID NO: 47 - DNA, 51 bp, synthetic construct; identical to SEQ ID NO: 7
  misc_feature 1..51: note = synthetic
  source 1..51: mol_type = other DNA

SEQ ID NO: 48 - protein, 17 aa, synthetic construct
  REGION 1..17: note = synthetic
AKYGSGYGKFYHYGLDV

SEQ ID NO: 49 - DNA, 372 bp, synthetic construct
  misc_feature 1..372: note = synthetic
  source 1..372: mol_type = other DNA
gaagtacagcttgtagaatccggcggaggactggtacaacctggaagaagtcttagactgagttgcgcagctagtgggtttacattcgacgattacagcatgcattgggtgaggcaagctcctggtaaaggattggaatgggttagcgggatatcatggaactcaggaagcaagggatacgccgacagcgtgaaaggccgatttacaatatctagggacaacgcaaaaaactctctctaccttcaaatgaactctcttagggcagaagacacagcattgtattattgcgcaaaagacggcagtggttatggcaagttttatcattatggactggacgtgtggggacaagggacaacagtgacagtgagtagc

SEQ ID NO: 50 - protein, 124 aa, synthetic construct
  REGION 1..124: note = synthetic
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKDGSGYGKFYHYGLDVWGQGTTVTVSS

SEQ ID NO: 51 - DNA, 24 bp, synthetic construct
  misc_feature 1..24: note = synthetic
  source 1..24: mol_type = other DNA
gggtttacattcgacgattacagc

SEQ ID NO: 52 - protein, 8 aa, synthetic construct
  REGION 1..8: note = synthetic
GFTFDDYS

SEQ ID NO: 53 - DNA, 24 bp, synthetic construct
  misc_feature 1..24: note = synthetic
  source 1..24: mol_type = other DNA
atatcatggaactcaggaagcaag

SEQ ID NO: 54 - protein, 8 aa, synthetic construct
  REGION 1..8: note = synthetic
ISWNSGSK

SEQ ID NO: 55 - DNA, 51 bp, synthetic construct
  misc_feature 1..51: note = synthetic
  source 1..51: mol_type = other DNA
gcaaaagacggcagtggttatggcaagttttatcattatggactggacgtg

SEQ ID NO: 56 - protein, 17 aa, synthetic construct
  REGION 1..17: note = synthetic
AKDGSGYGKFYHYGLDV

SEQ ID NO: 57 - DNA, 372 bp, synthetic construct
  misc_feature 1..372: note = synthetic
  source 1..372: mol_type = other DNA
gaagtacagcttgtagaatccggcggaggactggtacaacctggaagaagtcttagactgagttgcgcagctagtgggtttacattcgacgattacagcatgcattgggtgaggcaagctcctggtaaaggattggaatgggttagcgggatatcatggaactcaggaagcaagggatacgccgacagcgtgaaaggccgatttacaatatctagggacaacgcaaaaaactctctctaccttcaaatgaactctcttagggcagaagacacagcattgtattattgcgcaaaatacggcagtggttatggcaagttttattattatggactggacgtgtggggacaagggacaacagtgacagtgagtagc

SEQ ID NO: 58 - protein, 124 aa, synthetic construct
  REGION 1..124: note = synthetic
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYYYGLDVWGQGTTVTVSS

SEQ ID NO: 59 - DNA, 24 bp, synthetic construct
  misc_feature 1..24: note = synthetic
  source 1..24: mol_type = other DNA
gggtttacattcgacgattacagc

SEQ ID NO: 60 - protein, 8 aa, synthetic construct
  REGION 1..8: note = synthetic
GFTFDDYS

SEQ ID NO: 61 - DNA, 24 bp, synthetic construct
  misc_feature 1..24: note = synthetic
  source 1..24: mol_type = other DNA
atatcatggaactcaggaagcaag

SEQ ID NO: 62 - protein, 8 aa, synthetic construct
  REGION 1..8: note = synthetic
ISWNSGSK

SEQ ID NO: 63 - DNA, 51 bp, synthetic construct
  misc_feature 1..51: note = synthetic
  source 1..51: mol_type = other DNA
gcaaaatacggcagtggttatggcaagttttattattatggactggacgtg

SEQ ID NO: 64 - protein, 17 aa, synthetic construct
  REGION 1..17: note = synthetic
AKYGSGYGKFYYYGLDV

SEQ ID NO: 65 - DNA, 372 bp, synthetic construct
  misc_feature 1..372: note = synthetic
  source 1..372: mol_type = other DNA
gaagtacagcttgtagaatccggcggaggactggtacaacctggaagaagtcttagactgagttgcgcagctagtgggtttacattcgacgattacagcatgcattgggtgaggcaagctcctggtaaaggattggaatgggttagcgggatatcatggaactcaggaagcaagggatacgccgacagcgtgaaaggccgatttacaatatctagggacaacgcaaaaaactctctctaccttcaaatgaactctcttagggcagaagacacagcattgtattattgcgcaaaatacggcagtggttatggcaagttttatcattatggaatggacgtgtggggacaagggacaacagtgacagtgagtagc

SEQ ID NO: 66 - protein, 124 aa, synthetic construct
  REGION 1..124: note = synthetic
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGMDVWGQGTTVTVSS

SEQ ID NO: 67 - DNA, 24 bp, synthetic construct
  misc_feature 1..24: note = synthetic
  source 1..24: mol_type = other DNA
gggtttacattcgacgattacagc

SEQ ID NO: 68 - protein, 8 aa, synthetic construct
  REGION 1..8: note = synthetic
GFTFDDYS

SEQ ID NO: 69 - DNA, 24 bp, synthetic construct
  misc_feature 1..24: note = synthetic
  source 1..24: mol_type = other DNA
atatcatggaactcaggaagcaag

SEQ ID NO: 70 - protein, 8 aa, synthetic construct
  REGION 1..8: note = synthetic
ISWNSGSK

SEQ ID NO: 71 - DNA, 51 bp, synthetic construct
  misc_feature 1..51: note = synthetic
  source 1..51: mol_type = other DNA
gcaaaatacggcagtggttatggcaagttttatcattatggaatggacgtg

SEQ ID NO: 72 - protein, 17 aa, synthetic construct
  REGION 1..17: note = synthetic
AKYGSGYGKFYHYGMDV

SEQ ID NO: 73 - DNA, 372 bp, synthetic construct
  misc_feature 1..372: note = synthetic
  source 1..372: mol_type = other DNA
gaagtacagcttgtagaatccggcggaggactggtacaacctggaagaagtcttagactgagttgcgcagctagtgggtttacattcgacgattacagcatgcattgggtgaggcaagctcctggtaaaggattggaatgggttagcgggatatcatggaactcaggaagcatcggatacgccgacagcgtgaaaggccgatttacaatatctagggacaacgcaaaaaactctctctaccttcaaatgaactctcttagggcagaagacacagcattgtattattgcgcaaaagacggcagtggttatggcaagttttatcattatggactggacgtgtggggacaagggacaacagtgacagtgagtagc

SEQ ID NO: 74 - protein, 124 aa, synthetic construct
  REGION 1..124: note = synthetic
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKDGSGYGKFYHYGLDVWGQGTTVTVSS

SEQ ID NO: 75 - DNA, 24 bp, synthetic construct
  misc_feature 1..24: note = synthetic
  source 1..24: mol_type = other DNA
gggtttacattcgacgattacagc

SEQ ID NO: 76 - protein, 8 aa, synthetic construct
  REGION 1..8: note = synthetic
GFTFDDYS

SEQ ID NO: 77 - DNA, 24 bp, synthetic construct
  misc_feature 1..24: note = synthetic
  source 1..24: mol_type = other DNA
atatcatggaactcaggaagcatc

SEQ ID NO: 78 - protein, 8 aa, synthetic construct
  REGION 1..8: note = synthetic
ISWNSGSI

SEQ ID NO: 79 - DNA, 51 bp, synthetic construct; identical to SEQ ID NO: 55
  misc_feature 1..51: note = synthetic
  source 1..51: mol_type = other DNA

SEQ ID NO: 80 - protein, 17 aa, synthetic construct
  REGION 1..17: note = synthetic
AKDGSGYGKFYHYGLDV

SEQ ID NO: 81 - DNA, 372 bp, synthetic construct
  misc_feature 1..372: note = synthetic
  source 1..372: mol_type = other DNA
gaagtacagcttgtagaatccggcggaggactggtacaacctggaagaagtcttagactgagttgcgcagctagtgggtttacattcgacgattacagcatgcattgggtgaggcaagctcctggtaaaggattggaatgggttagcgggatatcatggaactcaggaagcatcggatacgccgacagcgtgaaaggccgatttacaatatctagggacaacgcaaaaaactctctctaccttcaaatgaactctcttagggcagaagacacagcattgtattattgcgcaaaatacggcagtggttatggcaagttttattattatggactggacgtgtggggacaagggacaacagtgacagtgagtagc

SEQ ID NO: 82 - protein, 124 aa, synthetic construct
  REGION 1..124: note = synthetic
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGLDVWGQGTTVTVSS

SEQ ID NO: 83 - DNA, 24 bp, synthetic construct
  misc_feature 1..24: note = synthetic
  source 1..24: mol_type = other DNA
gggtttacattcgacgattacagc

SEQ ID NO: 84 - protein, 8 aa, synthetic construct
  REGION 1..8: note = synthetic
GFTFDDYS

SEQ ID NO: 85 - DNA, 24 bp, synthetic construct
  misc_feature 1..24: note = synthetic
  source 1..24: mol_type = other DNA
atatcatggaactcaggaagcatc

SEQ ID NO: 86 - protein, 8 aa, synthetic construct
  REGION 1..8: note = synthetic
ISWNSGSI

SEQ ID NO: 87 - DNA, 51 bp, synthetic construct; identical to SEQ ID NO: 63
  misc_feature 1..51: note = synthetic
  source 1..51: mol_type = other DNA

SEQ ID NO: 88 - protein, 17 aa, synthetic construct
  REGION 1..17: note = synthetic
AKYGSGYGKFYYYGLDV

SEQ ID NO: 89 - DNA, 372 bp, synthetic construct
  misc_feature 1..372: note = synthetic
  source 1..372: mol_type = other DNA
gaagtacagcttgtagaatccggcggaggactggtacaacctggaagaagtcttagactgagttgcgcagctagtgggtttacattcgacgattacagcatgcattgggtgaggcaagctcctggtaaaggattggaatgggttagcgggatatcatggaactcaggaagcatcggatacgccgacagcgtgaaaggccgatttacaatatctagggacaacgcaaaaaactctctctaccttcaaatgaactctcttagggcagaagacacagcattgtattattgcgcaaaatacggcagtggttatggcaagttttatcattatggaatggacgtgtggggacaagggacaacagtgacagtgagtagc

SEQ ID NO: 90 - protein, 124 aa, synthetic construct
  REGION 1..124: note = synthetic
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYHYGMDVWGQGTTVTVSS

SEQ ID NO: 91 - DNA, 24 bp, synthetic construct
  misc_feature 1..24: note = synthetic
  source 1..24: mol_type = other DNA
gggtttacattcgacgattacagc

SEQ ID NO: 92 - protein, 8 aa, synthetic construct
  REGION 1..8: note = synthetic
GFTFDDYS

SEQ ID NO: 93 - DNA, 24 bp, synthetic construct
  misc_feature 1..24: note = synthetic
  source 1..24: mol_type = other DNA
atatcatggaactcaggaagcatc

SEQ ID NO: 94 - protein, 8 aa, synthetic construct
  REGION 1..8: note = synthetic
ISWNSGSI

SEQ ID NO: 95 - DNA, 51 bp, synthetic construct; identical to SEQ ID NO: 71
  misc_feature 1..51: note = synthetic
  source 1..51: mol_type = other DNA

SEQ ID NO: 96 - protein, 17 aa, synthetic construct
  REGION 1..17: note = synthetic
AKYGSGYGKFYHYGMDV

SEQ ID NO: 97 - DNA, 372 bp, synthetic construct
  misc_feature 1..372: note = synthetic
  source 1..372: mol_type = other DNA
gaagtacagcttgtagaatccggcggaggactggtacaacctggaagaagtcttagactgagttgcgcagctagtgggtttacattcgacgattacagcatgcattgggtgaggcaagctcctggtaaaggattggaatgggttagcgggatatcatggaactcaggaagcaagggatacgccgacagcgtgaaaggccgatttacaatatctagggacaacgcaaaaaactctctctaccttcaaatgaactctcttagggcagaagacacagcattgtattattgcgcaaaagacggcagtggttatggcaagttttattattatggactggacgtgtggggacaagggacaacagtgacagtgagtagc

SEQ ID NO: 98 - protein, 124 aa, synthetic construct
  REGION 1..124: note = synthetic
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKDGSGYGKFYYYGLDVWGQGTTVTVSS

SEQ ID NO: 99 - DNA, 24 bp, synthetic construct
  misc_feature 1..24: note = synthetic
  source 1..24: mol_type = other DNA
gggtttacattcgacgattacagc

SEQ ID NO: 100 - protein, 8 aa, synthetic construct
  REGION 1..8: note = synthetic
GFTFDDYS

SEQ ID NO: 101 - DNA, 24 bp, synthetic construct
  misc_feature 1..24: note = synthetic
  source 1..24: mol_type = other DNA
atatcatggaactcaggaagcaag

SEQ ID NO: 102 - protein, 8 aa, synthetic construct
  REGION 1..8: note = synthetic
ISWNSGSK

SEQ ID NO: 103 - DNA, 51 bp, synthetic construct
  misc_feature 1..51: note = synthetic
  source 1..51: mol_type = other DNA
gcaaaagacggcagtggttatggcaagttttattattatggactggacgtg

SEQ ID NO: 104 - protein, 17 aa, synthetic construct
  REGION 1..17: note = synthetic
AKDGSGYGKFYYYGLDV

SEQ ID NO: 105 - DNA, 372 bp, synthetic construct
  misc_feature 1..372: note = synthetic
  source 1..372: mol_type = other DNA
gaagtacagcttgtagaatccggcggaggactggtacaacctggaagaagtcttagactgagttgcgcagctagtgggtttacattcgacgattacagcatgcattgggtgaggcaagctcctggtaaaggattggaatgggttagcgggatatcatggaactcaggaagcaagggatacgccgacagcgtgaaaggccgatttacaatatctagggacaacgcaaaaaactctctctaccttcaaatgaactctcttagggcagaagacacagcattgtattattgcgcaaaagacggcagtggttatggcaagttttatcattatggaatggacgtgtggggacaagggacaacagtgacagtgagtagc

SEQ ID NO: 106 - protein, 124 aa, synthetic construct
  REGION 1..124: note = synthetic
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKDGSGYGKFYHYGMDVWGQGTTVTVSS

SEQ ID NO: 107 - DNA, 24 bp, synthetic construct
  misc_feature 1..24: note = synthetic
  source 1..24: mol_type = other DNA
gggtttacattcgacgattacagc

SEQ ID NO: 108 - protein, 8 aa, synthetic construct
  REGION 1..8: note = synthetic
GFTFDDYS

SEQ ID NO: 109 - DNA, 24 bp, synthetic construct
  misc_feature 1..24: note = synthetic
  source 1..24: mol_type = other DNA
atatcatggaactcaggaagcaag

SEQ ID NO: 110 - protein, 8 aa, synthetic construct
  REGION 1..8: note = synthetic
ISWNSGSK

SEQ ID NO: 111 - DNA, 51 bp, synthetic construct
  misc_feature 1..51: note = synthetic
  source 1..51: mol_type = other DNA
gcaaaagacggcagtggttatggcaagttttatcattatggaatggacgtg

SEQ ID NO: 112 - protein, 17 aa, synthetic construct
  REGION 1..17: note = synthetic
AKDGSGYGKFYHYGMDV

SEQ ID NO: 113 - DNA, 372 bp, synthetic construct
  misc_feature 1..372: note = synthetic
  source 1..372: mol_type = other DNA
gaagtacagcttgtagaatccggcggaggactggtacaacctggaagaagtcttagactgagttgcgcagctagtgggtttacattcgacgattacagcatgcattgggtgaggcaagctcctggtaaaggattggaatgggttagcgggatatcatggaactcaggaagcaagggatacgccgacagcgtgaaaggccgatttacaatatctagggacaacgcaaaaaactctctctaccttcaaatgaactctcttagggcagaagacacagcattgtattattgcgcaaaatacggcagtggttatggcaagttttattattatggaatggacgtgtggggacaagggacaacagtgacagtgagtagc

SEQ ID NO: 114 - protein, 124 aa, synthetic construct
  REGION 1..124: note = synthetic
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYYYGMDVWGQGTTVTVSS

SEQ ID NO: 115 - DNA, 24 bp, synthetic construct
  misc_feature 1..24: note = synthetic
  source 1..24: mol_type = other DNA
gggtttacattcgacgattacagc

SEQ ID NO: 116 - protein, 8 aa, synthetic construct
  REGION 1..8: note = synthetic
GFTFDDYS

SEQ ID NO: 117 - DNA, 24 bp, synthetic construct
  misc_feature 1..24: note = synthetic
  source 1..24: mol_type = other DNA
atatcatggaactcaggaagcaag

SEQ ID NO: 118 - protein, 8 aa, synthetic construct
  REGION 1..8: note = synthetic
ISWNSGSK

SEQ ID NO: 119 - DNA, 51 bp, synthetic construct
  misc_feature 1..51: note = synthetic
  source 1..51: mol_type = other DNA
gcaaaatacggcagtggttatggcaagttttattattatggaatggacgtg

SEQ ID NO: 120 - protein, 17 aa, synthetic construct
  REGION 1..17: note = synthetic
AKYGSGYGKFYYYGMDV

SEQ ID NO: 121 - DNA, 372 bp, synthetic construct
  misc_feature 1..372: note = synthetic
  source 1..372: mol_type = other DNA
gaagtacagcttgtagaatccggcggaggactggtacaacctggaagaagtcttagactgagttgcgcagctagtgggtttacattcgacgattacagcatgcattgggtgaggcaagctcctggtaaaggattggaatgggttagcgggatatcatggaactcaggaagcatcggatacgccgacagcgtgaaaggccgatttacaatatctagggacaacgcaaaaaactctctctaccttcaaatgaactctcttagggcagaagacacagcattgtattattgcgcaaaagacggcagtggttatggcaagttttattattatggactggacgtgtggggacaagggacaacagtgacagtgagtagc

SEQ ID NO: 122 - protein, 124 aa, synthetic construct
  REGION 1..124: note = synthetic
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKDGSGYGKFYYYGLDVWGQGTTVTVSS

SEQ ID NO: 123 - DNA, 24 bp, synthetic construct
  misc_feature 1..24: note = synthetic
  source 1..24: mol_type = other DNA
gggtttacattcgacgattacagc

SEQ ID NO: 124 - protein, 8 aa, synthetic construct
  REGION 1..8: note = synthetic
GFTFDDYS

SEQ ID NO: 125 - DNA, 24 bp, synthetic construct
  misc_feature 1..24: note = synthetic
  source 1..24: mol_type = other DNA
atatcatggaactcaggaagcatc

SEQ ID NO: 126 - protein, 8 aa, synthetic construct
  REGION 1..8: note = synthetic
ISWNSGSI

SEQ ID NO: 127 - DNA, 51 bp, synthetic construct; identical to SEQ ID NO: 103
  misc_feature 1..51: note = synthetic
  source 1..51: mol_type = other DNA

SEQ ID NO: 128 - protein, 17 aa, synthetic construct
  REGION 1..17: note = synthetic
AKDGSGYGKFYYYGLDV

SEQ ID NO: 129 - DNA, 372 bp, synthetic construct
  misc_feature 1..372: note = synthetic
  source 1..372: mol_type = other DNA
gaagtacagcttgtagaatccggcggaggactggtacaacctggaagaagtcttagactgagttgcgcagctagtgggtttacattcgacgattacagcatgcattgggtgaggcaagctcctggtaaaggattggaatgggttagcgggatatcatggaactcaggaagcatcggatacgccgacagcgtgaaaggccgatttacaatatctagggacaacgcaaaaaactctctctaccttcaaatgaactctcttagggcagaagacacagcattgtattattgcgcaaaagacggcagtggttatggcaagttttatcattatggaatggacgtgtggggacaagggacaacagtgacagtgagtagc

SEQ ID NO: 130 - protein, 124 aa, synthetic construct
  REGION 1..124: note = synthetic
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKDGSGYGKFYHYGMDVWGQGTTVTVSS

SEQ ID NO: 131 - DNA, 24 bp, synthetic construct
  misc_feature 1..24: note = synthetic
  source 1..24: mol_type = other DNA
gggtttacattcgacgattacagc

SEQ ID NO: 132 - protein, 8 aa, synthetic construct
  REGION 1..8: note = synthetic
GFTFDDYS

SEQ ID NO: 133 - DNA, 24 bp, synthetic construct
  misc_feature 1..24: note = synthetic
  source 1..24: mol_type = other DNA
atatcatggaactcaggaagcatc

SEQ ID NO: 134 - protein, 8 aa, synthetic construct
  REGION 1..8: note = synthetic
ISWNSGSI

SEQ ID NO: 135 - DNA, 51 bp, synthetic construct; identical to SEQ ID NO: 111
  misc_feature 1..51: note = synthetic
  source 1..51: mol_type = other DNA

SEQ ID NO: 136 - protein, 17 aa, synthetic construct
  REGION 1..17: note = synthetic
AKDGSGYGKFYHYGMDV

SEQ ID NO: 137 - DNA, 372 bp, synthetic construct
  misc_feature 1..372: note = synthetic
  source 1..372: mol_type = other DNA
gaagtacagcttgtagaatccggcggaggactggtacaacctggaagaagtcttagactgagttgcgcagctagtgggtttacattcgacgattacagcatgcattgggtgaggcaagctcctggtaaaggattggaatgggttagcgggatatcatggaactcaggaagcatcggatacgccgacagcgtgaaaggccgatttacaatatctagggacaacgcaaaaaactctctctaccttcaaatgaactctcttagggcagaagacacagcattgtattattgcgcaaaatacggcagtggttatggcaagttttattattatggaatggacgtgtggggacaagggacaacagtgacagtgagtagc

SEQ ID NO: 138 - protein, 124 aa, synthetic construct
  REGION 1..124: note = synthetic
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGMDVWGQGTTVTVSS

SEQ ID NO: 139 - DNA, 24 bp, synthetic construct
  misc_feature 1..24: note = synthetic
  source 1..24: mol_type = other DNA
gggtttacattcgacgattacagc

SEQ ID NO: 140 - protein, 8 aa, synthetic construct
  REGION 1..8: note = synthetic
GFTFDDYS

SEQ ID NO: 141 - DNA, 24 bp, synthetic construct
  misc_feature 1..24: note = synthetic
  source 1..24: mol_type = other DNA
atatcatggaactcaggaagcatc

SEQ ID NO: 142 - protein, 8 aa, synthetic construct
  REGION 1..8: note = synthetic
ISWNSGSI

SEQ ID NO: 143 - DNA, 51 bp, synthetic construct; identical to SEQ ID NO: 119
  misc_feature 1..51: note = synthetic
  source 1..51: mol_type = other DNA

SEQ ID NO: 144 - protein, 17 aa, synthetic construct
  REGION 1..17: note = synthetic
AKYGSGYGKFYYYGMDV

SEQ ID NO: 145 - DNA, 372 bp, synthetic construct
  misc_feature 1..372: note = synthetic
  source 1..372: mol_type = other DNA
gaagtacagcttgtagaatccggcggaggactggtacaacctggaagaagtcttagactgagttgcgcagctagtgggtttacattcgacgattacagcatgcattgggtgaggcaagctcctggtaaaggattggaatgggttagcgggatatcatggaactcaggaagcaagggatacgccgacagcgtgaaaggccgatttacaatatctagggacaacgcaaaaaactctctctaccttcaaatgaactctcttagggcagaagacacagcattgtattattgcgcaaaagacggcagtggttatggcaagttttattattatggaatggacgtgtggggacaagggacaacagtgacagtgagtagc

SEQ ID NO: 146 - protein, 124 aa, synthetic construct
  REGION 1..124: note = synthetic
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKDGSGYGKFYYYGMDVWGQGTTVTVSS

SEQ ID NO: 147 - DNA, 24 bp, synthetic construct
  misc_feature 1..24: note = synthetic
  source 1..24: mol_type = other DNA
gggtttacattcgacgattacagc

SEQ ID NO: 148 - protein, 8 aa, synthetic construct
  REGION 1..8: note = synthetic
GFTFDDYS

SEQ ID NO: 149 - DNA, 24 bp, synthetic construct
  misc_feature 1..24: note = synthetic
  source 1..24: mol_type = other DNA
atatcatggaactcaggaagcaag

SEQ ID NO: 150 - protein, 8 aa, synthetic construct
  REGION 1..8: note = synthetic
ISWNSGSK

SEQ ID NO: 151 - DNA, 51 bp, synthetic construct
  misc_feature 1..51: note = synthetic
  source 1..51: mol_type = other DNA
gcaaaagacggcagtggttatggcaagttttattattatggaatggacgtg

SEQ ID NO: 152 - protein, 17 aa, synthetic construct
  REGION 1..17: note = synthetic
AKDGSGYGKFYYYGMDV

SEQ ID NO: 153 - DNA, 372 bp, synthetic construct
  misc_feature 1..372: note = synthetic
  source 1..372: mol_type = other DNA
gaagtgcagctggtggaaagcggcggcggcctggtgcagccgggccgcagcctgcgcctgagctgcgcggcgagcggctttacctttgcggattataccatgcattgggtgcgccaggcgccgggcaaaggcctggaatgggtgagcgatattagctggaacagcggcagcattgcgtatgcggatagcgtgaaaggccgctttaccattagccgcgataacgcgaaaaacagcctgtatctgcagatgaacagcctgcgcaccgaagataccgcgttttattattgcgcgaaagatagccgcggctatggccattataaatatctgggcctggatgtgtggggccagggcaccaccgtgaccgtgagcagc

SEQ ID NO: 154 - protein, 124 aa, synthetic construct
  REGION 1..124: note = synthetic
EVQLVESGGGLVQPGRSLRLSCAASGFTFADYTMHWVRQAPGKGLEWVSDISWNSGSIAYADSVKGRFTISRDNAKNSLYLQMNSLRTEDTAFYYCAKDSRGYGHYKYLGLDVWGQGTTVTVSS

SEQ ID NO: 155 - DNA, 24 bp, synthetic construct
  misc_feature 1..24: note = synthetic
  source 1..24: mol_type = other DNA
ggctttacctttgcggattatacc

SEQ ID NO: 156 - protein, 8 aa, synthetic construct
  REGION 1..8: note = synthetic
GFTFADYT

SEQ ID NO: 157 - DNA, 24 bp, synthetic construct
  misc_feature 1..24: note = synthetic
  source 1..24: mol_type = other DNA
attagctggaacagcggcagcatt

SEQ ID NO: 158 - protein, 8 aa, synthetic construct
  REGION 1..8: note = synthetic
ISWNSGSI

SEQ ID NO: 159 - DNA, 51 bp, synthetic construct
  misc_feature 1..51: note = synthetic
  source 1..51: mol_type = other DNA
gcgaaagatagccgcggctatggccattataaatatctgggcctggatgtg

SEQ ID NO: 160 - protein, 17 aa, synthetic construct
  REGION 1..17: note = synthetic
AKDSRGYGHYKYLGLDV

SEQ ID NO: 161 - DNA, 324 bp, synthetic construct
  misc_feature 1..324: note = synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 162 - protein, 108 aa, synthetic construct
  REGION 1..108: note = synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 163 - DNA, 18 bp, synthetic construct
  misc_feature 1..18: note = synthetic
  source 1..18: mol_type = other DNA
cagagcattagcagctat

SEQ ID NO: 164 - protein, 6 aa, synthetic construct
  REGION 1..6: note = synthetic
QSISSY

SEQ ID NO: 165-166 - skipped (2 entries)

SEQ ID NO: 167 - DNA, 30 bp, synthetic construct
  misc_feature 1..30: note = synthetic
  source 1..30: mol_type = other DNA
caacagagttacagtacccctccgatcacc

SEQ ID NO: 168 - protein, 10 aa, synthetic construct
  REGION 1..10: note = synthetic
QQSYSTPPIT

SEQ ID NO: 169 - protein, 40 aa, synthetic construct
DEDEDEDEDEDEDEDERPLALWRSDRDRDRDRDRDRDRDR

SEQ ID NO: 170 - protein, 8 aa, synthetic construct
AARGPAIH

SEQ ID NO: 171 - protein, 8 aa, synthetic construct
AAYHLVSQ

SEQ ID NO: 172 - protein, 8 aa, synthetic construct
AGLGISST

SEQ ID NO: 173 - protein, 8 aa, synthetic construct
AGLGVVER

SEQ ID NO: 174 - protein, 4 aa, synthetic construct
ALAL

SEQ ID NO: 175 - protein, 8 aa, synthetic construct
ALFFSSPP

SEQ ID NO: 176 - protein, 8 aa, synthetic construct
ALFKSSFP

SEQ ID NO: 177 - protein, 7 aa, synthetic construct
ALLLALL

SEQ ID NO: 178 - protein, 8 aa, synthetic construct
AQFVLTEG

SEQ ID NO: 179 - protein, 8 aa, synthetic construct
AQNLLGMV

SEQ ID NO: 180 - protein, 8 aa, synthetic construct
AVGLLAPP

SEQ ID NO: 181 - protein, 4 aa, synthetic construct
DAFK

SEQ ID NO: 182 - protein, 4 aa, synthetic construct
DEVD

SEQ ID NO: 183 - protein, 5 aa, synthetic construct
DEVDP

SEQ ID NO: 184 - protein, 6 aa, synthetic construct
DPRSFL

SEQ ID NO: 185 - protein, 8 aa, synthetic construct
DVAQFVLT

SEQ ID NO: 186 - protein, 4 aa, synthetic construct
DVLK

SEQ ID NO: 187 - protein, 8 aa, synthetic construct
DWLYWPGI

SEQ ID NO: 188 - protein, 7 aa, synthetic construct
EDDDDKA

SEQ ID NO: 189 - protein, 6 aa, synthetic construct
  SITE 3: note = Citrulline
  SITE 5: note = Hypofluorous acid modified tyrosine
EPXGYL

SEQ ID NO: 190 - protein, 8 aa, synthetic construct
EPQALAMS

SEQ ID NO: 191 - protein, 8 aa, synthetic construct
ESLPVVAV

SEQ ID NO: 192 - protein, 8 aa, synthetic construct
ESPAYYTA

SEQ ID NO: 193 - protein, 10 aa, synthetic construct
FPRPLGITGL

SEQ ID NO: 194 - protein, 8 aa, synthetic construct
FRLLDWQW

SEQ ID NO: 195 - protein, 4 aa, synthetic construct
GFLG

SEQ ID NO: 196 - protein, 10 aa, synthetic construct
GGAANLVRGG

SEQ ID NO: 197 - protein, 5 aa, synthetic construct
GGGRR

SEQ ID NO: 198 - protein, 8 aa, synthetic construct
GGPRGLPG

SEQ ID NO: 199 - protein, 10 aa, synthetic construct
GGQPSGMWGW

SEQ ID NO: 200 - protein, 7 aa, synthetic construct
GGSIDGR

SEQ ID NO: 201 - protein, 8 aa, synthetic construct
GGWHTGRN

SEQ ID NO: 202 - protein, 5 aa, synthetic construct
GIAGQ

SEQ ID NO: 203 - protein, 6 aa, synthetic construct
GKAFRR

SEQ ID NO: 204 - protein, 8 aa, synthetic construct
GPAGLYAQ

SEQ ID NO: 205 - protein, 8 aa, synthetic construct
GPAGMKGL

SEQ ID NO: 206 - protein, 8 aa, synthetic construct
GPEGLRVG

SEQ ID NO: 207 - protein, 8 aa, synthetic construct
GPLGIAGI

SEQ ID NO: 208 - protein, 7 aa, synthetic construct
GPLGVRG

SEQ ID NO: 209 - protein, 8 aa, synthetic construct
GPQGIAGQ

SEQ ID NO: 210 - protein, 8 aa, synthetic construct
GPQGLLGA

SEQ ID NO: 211 - protein, 6 aa, synthetic construct
GPRSFG

SEQ ID NO: 212 - protein, 7 aa, synthetic construct
GPRSFGL

SEQ ID NO: 213 - protein, 8 aa, synthetic construct
GPSHLVLT

SEQ ID NO: 214 - protein, 10 aa, synthetic construct
GVSQNYPIVG

SEQ ID NO: 215 - protein, 10 aa, synthetic construct
GVVQASCRLA

SEQ ID NO: 216 - protein, 6 aa, synthetic construct
GWEHDG

SEQ ID NO: 217 - protein, 6 aa, synthetic construct
HSSKLQ

SEQ ID NO: 218 - protein, 9 aa, synthetic construct
HSSKLQEDA

SEQ ID NO: 219 - protein, 7 aa, synthetic construct
HSSKLQL

SEQ ID NO: 220 - protein, 8 aa, synthetic construct
HTGRSGAL

SEQ ID NO: 221 - protein, 4 aa, synthetic construct
IDGR

SEQ ID NO: 222 - protein, 4 aa, synthetic construct
IEGR

SEQ ID NO: 223 - protein, 8 aa, synthetic construct
ILPRSPAF

SEQ ID NO: 224 - protein, 8 aa, synthetic construct
IPVSLRSG

SEQ ID NO: 225 - protein, 5 aa, synthetic construct
ISSGL

SEQ ID NO: 226 - protein, 6 aa, synthetic construct
ISSGLL

SEQ ID NO: 227 - protein, 7 aa, synthetic construct
ISSGLLS

SEQ ID NO: 228 - protein, 8 aa, synthetic construct
ISSGLLSS

SEQ ID NO: 229 - protein, 7 aa, synthetic construct
ISSGLSS

SEQ ID NO: 230 - protein, 8 aa, synthetic construct
KGSGDVEG

SEQ ID NO: 231 - protein, 9 aa, synthetic construct
KQEQNPGST

SEQ ID NO: 232 - protein, 8 aa, synthetic construct
KRALGLPG

SEQ ID NO: 233 - protein, 8 aa, synthetic construct
LAAPLGLL

SEQ ID NO: 234 - protein, 9 aa, synthetic construct
LAPLGLQRR

SEQ ID NO: 235 - protein, 8 aa, synthetic construct
LAQKLKSS

SEQ ID NO: 236 - protein, 8 aa, synthetic construct
LAQRLRSS

SEQ ID NO: 237 - protein, 5 aa, synthetic construct
LEATA

SEQ ID NO: 238 - protein, 8 aa, synthetic construct
LKAAPRWA

SEQ ID NO: 239 - protein, 8 aa, synthetic construct
LLAPSHRA

SEQ ID NO: 240 - protein, 8 aa, synthetic construct
LPGGLSPW

SEQ ID NO: 241 - protein, 8 aa, synthetic construct
LSGRSANI

SEQ ID NO: 242 - protein, 8 aa, synthetic construct
LSGRSANP

SEQ ID NO: 243 - protein, 8 aa, synthetic construct
LSGRSDDH

SEQ ID NO: 244 - protein, 8 aa, synthetic construct
LSGRSDIH

SEQ ID NO: 245 - protein, 8 aa, synthetic construct
LSGRSDNH

SEQ ID NO: 246 - protein, 8 aa, synthetic construct
LSGRSDNI

SEQ ID NO: 247 - protein, 8 aa, synthetic construct
LSGRSDNP

SEQ ID NO: 248 - protein, 8 aa, synthetic construct
LSGRSDQG

SEQ ID NO: 249 - protein, 8 aa, synthetic construct
LSGRSDQH

SEQ ID NO: 250 - protein, 8 aa, synthetic construct
LSGRSDTH

SEQ ID NO: 251 - protein, 8 aa, synthetic construct
LSGRSDYH

SEQ ID NO: 252 - protein, 8 aa, synthetic construct
LSGRSGNH

SEQ ID NO: 253 - protein, 10 aa, synthetic construct
LVLASSSFGY

SEQ ID NO: 254 - protein, 8 aa, synthetic construct
MDAFLESS

SEQ ID NO: 255 - protein, 8 aa, synthetic construct
MGLFSEAG

SEQ ID NO: 256 - protein, 8 aa, synthetic construct
MIAPVAYR

SEQ ID NO: 257 - protein, 8 aa, synthetic construct
MVLGRSLL

SEQ ID NO: 258 - protein, 12 aa, synthetic construct
NTLSGRSENHSG

SEQ ID NO: 259 - protein, 12 aa, synthetic construct
NTLSGRSGNHGS

SEQ ID NO: 260 - protein, 8 aa, synthetic construct
PAGLWLDP

SEQ ID NO: 261 - protein, 8 aa, synthetic construct
PGGPAGIG

SEQ ID NO: 262 - protein, 5 aa, synthetic construct
  SITE 4: note = Ethyl phenylalanine
PICFF

SEQ ID NO: 263 - protein, 6 aa, synthetic construct
  SITE 4: note = Methyl cysteine
PLGCAG

SEQ ID NO: 264 - protein, 4 aa, synthetic construct
PLGL

SEQ ID NO: 265 - protein, 6 aa, synthetic construct
PLGLAG

SEQ ID NO: 266 - protein, 6 aa, synthetic construct
  VARIANT 6: note = Any amino acid
PLGLAX

SEQ ID NO: 267 - protein, 6 aa, synthetic construct
PLGLWA

SEQ ID NO: 268 - protein, 7 aa, synthetic construct
PLGLWSQ

SEQ ID NO: 269 - protein, 8 aa, synthetic construct
PLTGRSGG

SEQ ID NO: 270 - protein, 5 aa, synthetic construct
PMAKK

SEQ ID NO: 271 - protein, 6 aa, synthetic construct
PPRSFL

SEQ ID NO: 272 - protein, 8 aa, synthetic construct
PRFRIIGG

SEQ ID NO: 273 - protein, 8 aa, synthetic construct
PVGYTSSL

SEQ ID NO: 274 - protein, 8 aa, synthetic construct
PVQPIGPQ

SEQ ID NO: 275 - protein, 8 aa, synthetic construct
QALAMSAI

SEQ ID NO: 276 - protein, 8 aa, synthetic construct
QGRAITFI

SEQ ID NO: 277 - protein, 8 aa, synthetic construct
QNQALRMA

SEQ ID NO: 278 - protein, 4 aa, synthetic construct
RGPA

SEQ ID NO: 279 - protein, 8 aa, synthetic construct
RGPAFNPM

SEQ ID NO: 280 - protein, 8 aa, synthetic construct
RGPATPIM

SEQ ID NO: 281 - protein, 14 aa, synthetic construct
RKSSIIIRMRDVVL

SEQ ID NO: 282 - protein, 7 aa, synthetic construct
RLQLKAC

SEQ ID NO: 283 - protein, 6 aa, synthetic construct
RLQLKL

SEQ ID NO: 284 - protein, 8 aa, synthetic construct
RMHLRSLG

SEQ ID NO: 285 - protein, 8 aa, synthetic construct
RPSPMWAY

SEQ ID NO: 286 - protein, 8 aa, synthetic construct
RQARVVNG

SEQ ID NO: 287 - protein, 8 aa, synthetic construct
SAGFSLPA

SEQ ID NO: 288 - protein, 8 aa, synthetic construct
SAPAVESE

SEQ ID NO: 289 - protein, 8 aa, synthetic construct
SARGPSRW

SEQ ID NO: 290 - protein, 9 aa, synthetic construct
SGEPAYYTA

SEQ ID NO: 291 - protein, 8 aa, synthetic construct
SGGPLGVR

SEQ ID NO: 292 - protein, 10 aa, synthetic construct
SGRIGFLRTA

SEQ ID NO: 293 - protein, 5 aa, synthetic construct
SGRSA

SEQ ID NO: 294 - protein, 9 aa, synthetic construct
SGRSANPRG

SEQ ID NO: 295 - protein, 8 aa, synthetic construct
SMLRSMPL

SEQ ID NO: 296 - protein, 8 aa, synthetic construct
SPLPLRVP

SEQ ID NO: 297 - protein, 8 aa, synthetic construct
SPLTGRSG

SEQ ID NO: 298 - protein, 8 aa, synthetic construct
SPRSIMLA

SEQ ID NO: 299 - protein, 8 aa, synthetic construct
SSRGPAYL

SEQ ID NO: 300 - protein, 9 aa, synthetic construct
SSRHRRALD

SEQ ID NO: 301 - protein, 15 aa, synthetic construct
SSSFDKGKYKKGDDA

SEQ ID NO: 302 - protein, 15 aa, synthetic construct
SSSFDKGKYKRGDDA

SEQ ID NO: 303 - protein, 8 aa, synthetic construct
STFPFGMF

SEQ ID NO: 304 - protein, 8 aa, synthetic construct
TARGPSFK

SEQ ID NO: 305 - protein, 8 aa, synthetic construct
TGRGPSWV

SEQ ID NO: 306 - protein, 8 aa, synthetic construct
TSGRSANP

SEQ ID NO: 307 - protein, 12 aa, synthetic construct
TSTSGRSANPRG

SEQ ID NO: 308 - protein, 8 aa, synthetic construct
VAGRSMRP

SEQ ID NO: 309 - protein, 8 aa, synthetic construct
VAQFVLTE

SEQ ID NO: 310 - protein, 10 aa, synthetic construct
VHMPLGFLGP

SEQ ID NO: 311 - protein, 8 aa, synthetic construct
VPLSLYSG

SEQ ID NO: 312 - protein, 8 aa, synthetic construct
VVPEGRRS

SEQ ID NO: 313 - protein, 8 aa, synthetic construct
WATPRPMR

SEQ ID NO: 314 - protein, 8 aa, synthetic construct
YGAGLGVV

SEQ ID NO: 315 - protein, 5 aa, synthetic construct
GGGGS

SEQ ID NO: 316 - protein, 4 aa, synthetic construct
GGGS

SEQ ID NO: 317 - protein, 5 aa, synthetic construct
GSGGS

SEQ ID NO: 318 - protein, 10 aa, synthetic construct
GGGGSGGGGS

SEQ ID NO: 319 - protein, 15 aa, synthetic construct
GGGGSGGGGSGGGGS

SEQ ID NO: 320 - protein, 20 aa, synthetic construct
GGGGSGGGGSGGGGSGGGGS

SEQ ID NO: 321 - protein, 15 aa, synthetic construct
GGGKSGGGKSGGGKS

SEQ ID NO: 322 - protein, 15 aa, synthetic construct
GGGKSGGKGSGKGGS

SEQ ID NO: 323 - protein, 4 aa, synthetic construct
GGGS

SEQ ID NO: 324 - protein, 5 aa, synthetic construct
GGGSG

SEQ ID NO: 325 - protein, 15 aa, synthetic construct
GGKGSGGKGSGGKGS

SEQ ID NO: 326 - protein, 13 aa, synthetic construct
GGSGGGGSGGGGS

SEQ ID NO: 327 - protein, 6 aa, synthetic construct
GGSGGS

SEQ ID NO: 328 - protein, 11 aa, synthetic construct
GGSGGSGGSGS

SEQ ID NO: 329 - protein, 5 aa, synthetic construct
GSGGG

SEQ ID NO: 330 - protein, 5 aa, synthetic construct
GSGSG

SEQ ID NO: 331 - protein, 4 aa, synthetic construct
GSSG

SEQ ID NO: 332 - protein, 10 aa, synthetic construct
GSSGGSGGSG

SEQ ID NO: 333 - protein, 11 aa, synthetic construct
GSSGGSGGSGG

SEQ ID NO: 334 - protein, 12 aa, synthetic construct
GSSGGSGGSGGS

SEQ ID NO: 335 - protein, 13 aa, synthetic construct
GSSGGSGGSGGSG

SEQ ID NO: 336 - protein, 16 aa, synthetic construct
GSSGGSGGSGGSGGGS

SEQ ID NO: 337 - protein, 11 aa, synthetic construct
GSSGGSGGSGS

SEQ ID NO: 338 - protein, 5 aa, synthetic construct
GSSGT

SEQ ID NO: 339 - protein, 5 aa, synthetic construct
GSSSG

SEQ ID NO: 340 - protein, 6 aa, synthetic construct
QGQSGQ

SEQ ID NO: 341 - protein, 7 aa, synthetic construct
QGQSGQG

SEQ ID NO: 342 - protein, 6 aa, synthetic construct
QGQSGS

SEQ ID NO: 343 - protein, 4 aa, synthetic construct
QSGQ

SEQ ID NO: 344 - protein, 5 aa, synthetic construct
QSGQG

SEQ ID NO: 345 - protein, 4 aa, synthetic construct
QSGS

SEQ ID NO: 346 - protein, 4 aa, synthetic construct
SGQG

SEQ ID NO: 347 - protein, 35 aa, synthetic construct
GGGISSGLLSGRSDNHGGGISSGLLSGRSDNHGGS

SEQ ID NO: 348 - protein, 70 aa, synthetic construct
GGGISSGLLSGRSDNHGGGISSGLLSGRSDNHGGSGGGISSGLLSGRSDNHGGGISSGLLSGRSDNHGGS

SEQ ID NO: 349 - protein, 35 aa, synthetic construct
GGSGGSIPVSLRSGGGISSGLLSGRSDNHGGSGGS

SEQ ID NO: 350 - protein, 35 aa, synthetic construct
GGSGGSVPLSLYSGGGISSGLLSGRSDNHGGSGGS

SEQ ID NO: 351 - protein, 35 aa, synthetic construct
GGSHPVGLLARGGGHPVGLLARGGGHPVGLLARGS

SEQ ID NO: 352 - protein, 36 aa, synthetic construct
GGSHPVGLLARGGGHPVGLLARGGSGRSAGGSGRSA

SEQ ID NO: 353 - protein, 18 aa, synthetic construct
AVGLLAPPGGLSGRSANI

SEQ ID NO: 354 - protein, 18 aa, synthetic construct
AVGLLAPPGGLSGRSANP

SEQ ID NO: 355 - protein, 18 aa, synthetic construct
AVGLLAPPGGLSGRSDDH

SEQ ID NO: 356 - protein, 18 aa, synthetic construct
AVGLLAPPGGLSGRSDIH

SEQ ID NO: 357 - protein, 18 aa, synthetic construct
AVGLLAPPGGLSGRSDNH

SEQ ID NO: 358 - protein, 18 aa, synthetic construct
AVGLLAPPGGLSGRSDNI

SEQ ID NO: 359 - protein, 18 aa, synthetic construct
AVGLLAPPGGLSGRSDNP

SEQ ID NO: 360 - protein, 18 aa, synthetic construct
AVGLLAPPGGLSGRSDQH

SEQ ID NO: 361 - protein, 18 aa, synthetic construct
AVGLLAPPGGLSGRSDTH

SEQ ID NO: 362 - protein, 18 aa, synthetic construct
AVGLLAPPGGLSGRSDYH

SEQ ID NO: 363 - protein, 22 aa, synthetic construct
AVGLLAPPGGTSTSGRSANPRG

SEQ ID NO: 364 - protein, 17 aa, synthetic construct
AVGLLAPPSGRSANPRG

SEQ ID NO: 365 - protein, 18 aa, synthetic construct
AVGLLAPPTSGRSANPRG

SEQ ID NO: 366 - protein, 30 aa, synthetic construct
GGALFKSSFPGPAGLYAQPLAQKLKSSGGK

SEQ ID NO: 367 - protein, 33 aa, synthetic construct
GGGGSGGGGSGGGGSFVGGTGGGGSGGGGSGGS

SEQ ID NO: 368 - protein, 34 aa, synthetic construct
GGGGSGGGGSGGGGSISSGLLSGRSDNHGGSGGS

SEQ ID NO: 369 - protein, 34 aa, synthetic construct
GGGGSGGGGSGGGGSVPLSLYSGGGSGGSGGSGS

SEQ ID NO: 370 - protein, 35 aa, synthetic construct
GGGGSGGGGSGPLGLWSQGGGGSGGGGSGGGGSGG

SEQ ID NO: 371 - protein, 33 aa, synthetic construct
GGGGSGGGGSKKAAPGGGGSGGGGSGGGGSGGS

SEQ ID NO: 372 - protein, 33 aa, synthetic construct
GGGGSGGGGSKKAAPVNGGGGGSGGGGSGGGGS

SEQ ID NO: 373 - protein, 33 aa, synthetic construct
GGGGSGGGGSPMAKKGGGGSGGGGSGGGGSGGS

SEQ ID NO: 374 - protein, 33 aa, synthetic construct
GGGGSGGGGSPMAKKVNGGGGGSGGGGSGGGGS

SEQ ID NO: 375 - protein, 33 aa, synthetic construct
GGGGSGGGGSQARAKGGGGSGGGGSGGGGSGGS

SEQ ID NO: 376 - protein, 33 aa, synthetic construct
GGGGSGGGGSQARAKVNGGGGGSGGGGSGGGGS

SEQ ID NO: 377 - protein, 33 aa, synthetic construct
GGGGSGGGGSRQARVVNGGGGGSGGGGSGGGGS

SEQ ID NO: 378 - protein, 41 aa, synthetic construct
GGGGSGGGGSRQARVVNGGGGGSVPLSLYSGGGGGSGGGGS

SEQ ID NO: 379 - protein, 36 aa, synthetic construct
GGGGSGGGGSRQARVVNSVPLSLYSGGGGGSGGGGS

SEQ ID NO: 380 - protein, 33 aa, synthetic construct
GGGGSGGGGSVHMPLGFLGPGGGGSGGGGSGGS

SEQ ID NO: 381 - protein, 28 aa, synthetic construct
GGGGSVHMPLGFLGPGRSRGSFPGGGGS

SEQ ID NO: 382 - protein, 33 aa, synthetic construct
GGGGSVHMPLGFLGPPMAKKGGGGSGGGGSGGS

SEQ ID NO: 383 - protein, 32 aa, synthetic construct
GGGGSVHMPLGFLGPRQARVVNGGGGSGGGGS

SEQ ID NO: 384 - protein, 34 aa, synthetic construct
GGGGSVHMPLGFLGPRQARVVNGGGGSGGGGSGG

SEQ ID NO: 385 - protein, 30 aa, synthetic construct
GGPLAQKLKSSALFKSSFPGPAGLYAQGGR

SEQ ID NO: 386 - protein, 19 aa, synthetic construct
GLSGRSDNHGGAVGLLAPP

SEQ ID NO: 387 - protein, 21 aa, synthetic construct
GLSGRSDNHGGVHMPLGFLGP

SEQ ID NO: 388 - protein, 13 aa, synthetic construct
ISSGLLSGRSANI

SEQ ID NO: 389 - protein, 13 aa, synthetic construct
ISSGLLSGRSANP

SEQ ID NO: 390 - protein, 15 aa, synthetic construct
ISSGLLSGRSANPRG

SEQ ID NO: 391 - protein, 13 aa, synthetic construct
ISSGLLSGRSDDH

SEQ ID NO: 392 - protein, 13 aa, synthetic construct
ISSGLLSGRSDIH

SEQ ID NO: 393 - protein, 13 aa, synthetic construct
ISSGLLSGRSDNH

SEQ ID NO: 394 - protein, 13 aa, synthetic construct
ISSGLLSGRSDNI

SEQ ID NO: 395 - protein, 13 aa, synthetic construct
ISSGLLSGRSDNP

SEQ ID NO: 396 - protein, 13 aa, synthetic construct
ISSGLLSGRSDQH

SEQ ID NO: 397 - protein, 13 aa, synthetic construct
ISSGLLSGRSDTH

SEQ ID NO: 398 - protein, 13 aa, synthetic construct
ISSGLLSGRSDYH

SEQ ID NO: 399 - protein, 13 aa, synthetic construct
ISSGLLSGRSGNH

SEQ ID NO: 400 - protein, 22 aa, synthetic construct
ISSGLLSSGGSGGSLSGRSDNH

SEQ ID NO: 401 - protein, 22 aa, synthetic construct
ISSGLLSSGGSGGSLSGRSGNH

SEQ ID NO: 402 - protein, 30 aa, synthetic construct
KGGPGGPAGIGPLAQRLRSSALFKSSFPGR

SEQ ID NO: 403 - protein, 30 aa, synthetic construct
KSGPGGPAGIGALFFSSPPLAQKLKSSGGR

SEQ ID NO: 404 - protein, 18 aa, synthetic construct
LSGRSDNHGGAVGLLAPP

SEQ ID NO: 405 - protein, 22 aa, synthetic construct
LSGRSDNHGGSGGSISSGLLSS

SEQ ID NO: 406 - protein, 22 aa, synthetic construct
LSGRSDNHGGSGGSQNQALRMA

SEQ ID NO: 407 - protein, 20 aa, synthetic construct
LSGRSDNHGGVHMPLGFLGP

SEQ ID NO: 408 - protein, 22 aa, synthetic construct
LSGRSGNHGGSGGSISSGLLSS

SEQ ID NO: 409 - protein, 22 aa, synthetic construct
LSGRSGNHGGSGGSQNQALRMA

SEQ ID NO: 410 - protein, 22 aa, synthetic construct
QNQALRMAGGSGGSLSGRSDNH

SEQ ID NO: 411 - protein, 22 aa, synthetic construct
QNQALRMAGGSGGSLSGRSGNH

SEQ ID NO: 412 - protein, 30 aa, synthetic construct
RGGALFKSSFPLAQKLKSSGPAGLYAQGGK

SEQ ID NO: 413 - protein, 30 aa, synthetic construct
RGGGPAGLYAQPLAQKLKSSALFKSSFPGG

SEQ ID NO: 414 - protein, 30 aa, synthetic construct
SGGFPRSGGSFNPRTFGSKRKRRGSRGGGG

SEQ ID NO: 415 - protein, 30 aa, synthetic construct
SGPLAQKLKSSGPAGLYAQALFKSSFPGSK

SEQ ID NO: 416 - protein, 22 aa, synthetic construct
TSTSGRSANPRGGGAVGLLAPP

SEQ ID NO: 417 - protein, 24 aa, synthetic construct
TSTSGRSANPRGGGVHMPLGFLGP

SEQ ID NO: 418 - protein, 20 aa, synthetic construct
VHMPLGFLGPGGLSGRSDNH

SEQ ID NO: 419 - protein, 24 aa, synthetic construct
VHMPLGFLGPGGTSTSGRSANPRG

SEQ ID NO: 420 - protein, 21 aa, synthetic construct
SGRSAGGGSGRSAGGGSGRSA

SEQ ID NO: 421 - protein, 35 aa, synthetic construct
HPVGLLARGGGHPVGLLARGGGSGRSAGGGSGRSA

SEQ ID NO: 422 - protein, 8 aa, synthetic construct
GPLGVRGK

SEQ ID NO: 423 - protein, 8 aa, synthetic construct
HPVGLLAR

SEQ ID NO: 424 - protein, 8 aa, synthetic construct
VPMSMRGG

SEQ ID NO: 425 - protein, 8 aa, synthetic construct
RPFSMIMG

SEQ ID NO: 426 - protein, 8 aa, synthetic construct
VPLSLTMG

SEQ ID NO: 427 - protein, 8 aa, synthetic construct
IPESLRAG

SEQ ID NO: 428 - protein, 5 aa, synthetic construct
ADAAP

SEQ ID NO: 429 - protein, 11 aa, synthetic construct
ADAAPTVSIFP

SEQ ID NO: 430 - protein, 12 aa, synthetic construct
ADAAPTVSIFPP

SEQ ID NO: 431 - protein, 6 aa, synthetic construct
AKTTAP

SEQ ID NO: 432 - protein, 13 aa, synthetic construct
AKTTAPSVYPLAP

SEQ ID NO: 433 - protein, 17 aa, synthetic construct
AKTTPKLEEGEFSEARV

SEQ ID NO: 434 - protein, 9 aa, synthetic construct
AKTTPKLGG

SEQ ID NO: 435 - protein, 6 aa, synthetic construct
AKTTPP

SEQ ID NO: 436 - protein, 13 aa, synthetic construct
AKTTPPSVTPLAP

SEQ ID NO: 437 - protein, 6 aa, synthetic construct
ASTKGP

SEQ ID NO: 438 - protein, 26 aa, synthetic construct
ASTKGPSVFPLAPASTKGPSVFPLAP

SEQ ID NO: 439 - protein, 14 aa, synthetic construct
EGKSSGSGSESKST

SEQ ID NO: 440 - protein, 15 aa, synthetic construct
GEGESGEGESGEGES

SEQ ID NO: 441 - protein, 20 aa, synthetic construct
GEGESGEGESGEGESGEGES

SEQ ID NO: 442 - protein, 15 aa, synthetic construct
GEGGSGEGGSGEGGS

SEQ ID NO: 443 - protein, 15 aa, synthetic construct
GENKVEYAPALMALS

SEQ ID NO: 444 - protein, 15 aa, synthetic construct
GGEGSGGEGSGGEGS

SEQ ID NO: 445 - protein, 15 aa, synthetic construct
GGGESGGEGSGEGGS

SEQ ID NO: 446 - protein, 15 aa, synthetic construct
GGGESGGGESGGGES

SEQ ID NO: 447 - protein, 4 aa, synthetic construct
GGSG

SEQ ID NO: 448 - protein, 5 aa, synthetic construct
GGSGG

SEQ ID NO: 449 - protein, 9 aa, synthetic construct
GGSGGGGSG

SEQ ID NO: 450 - protein, 15 aa, synthetic construct
GHEAAAVMQVQYPAS

SEQ ID NO: 451 - protein, 15 aa, synthetic construct
GKGGSGKGGSGKGGS

SEQ ID NO: 452 - protein, 15 aa, synthetic construct
GKGKSGKGKSGKGKS

SEQ ID NO: 453 - protein, 20 aa, synthetic construct
GKGKSGKGKSGKGKSGKGKS

SEQ ID NO: 454 - protein, 15 aa, synthetic construct
GKPGSGKPGSGKPGS

SEQ ID NO: 455 - protein, 21 aa, synthetic construct
GKPGSGKPGSGKPGSGKPSGS

SEQ ID NO: 456 - protein, 15 aa, synthetic construct
GPAKELTPLKEAKVS

SEQ ID NO: 457 - protein, 12 aa, synthetic construct
GSAGSAAGSGEF

SEQ ID NO: 458 - protein, 18 aa, synthetic construct
GSTSGSGKPGSGEGSTKG

SEQ ID NO: 459 - protein, 19 aa, synthetic construct
GTAAAGAGAAGGAAAGAAG

SEQ ID NO: 460 - protein, 19 aa, synthetic construct
GTSGSSGSGSGGSGSGGGG

SEQ ID NO: 461 - protein, 14 aa, synthetic construct
IRPRAIGGSKPRVA

SEQ ID NO: 462 - protein, 18 aa, synthetic construct
KESGSVSSEQLAQFRSLD

SEQ ID NO: 463 - protein, 15 aa, synthetic construct
KTTPKLEEGEFSEAR

SEQ ID NO: 464 - protein, 19 aa, synthetic construct
PRGASKSGSASQTGSAPGS

SEQ ID NO: 465 - protein, 6 aa, synthetic construct
QPKAAP

SEQ ID NO: 466 - protein, 13 aa, synthetic construct
QPKAAPSVTLFPP

SEQ ID NO: 467 - protein, 27 aa, synthetic construct
RADAAAAGGGGSGGGGSGGGGSGGGGS

SEQ ID NO: 468 - protein, 12 aa, synthetic construct
RADAAAAGGPGS

SEQ ID NO: 469 - protein, 6 aa, synthetic construct
RADAAP

SEQ ID NO: 470 - protein, 9 aa, synthetic construct
RADAAPTVS

SEQ ID NO: 471 - protein, 6 aa, synthetic construct
SAKTTP

SEQ ID NO: 472 - protein, 18 aa, synthetic construct
SAKTTPKLEEGEFSEARV

SEQ ID NO: 473 - protein, 10 aa, synthetic construct
SAKTTPKLGG

SEQ ID NO: 474 - protein, 14 aa, synthetic construct
STAGDTHLGGEDFD

SEQ ID NO: 475 - protein, 5 aa, synthetic construct
TVAAP

SEQ ID NO: 476 - protein, 12 aa, synthetic construct
TVAAPSVFIFPP

SEQ ID NO: 477 - protein, 24 aa, synthetic construct
TVAAPSVFIFPPTVAAPSVFIFPP

SEQ ID NO: 478 - protein, 20 aa, synthetic construct
GGGISSGLLSGRSDNHGGGS

SEQ ID NO: 479 - protein, 15 aa, synthetic construct
GGGHPVGLLARGGGS

SEQ ID NO: 480 - protein, 36 aa, synthetic construct
GGGSGGGSGGGGISSGLLSGRSDNHGGGSGGGSGGS

SEQ ID NO: 481 - protein, 36 aa, synthetic construct
GGGGISSGLLSGRSDNHGGGISSGLLSGRSDNHGGS

SEQ ID NO: 482 - protein, 36 aa, synthetic construct
GGGSGGSIPVSLRSGGGISSGLLSGRSDNHGGSGGS

SEQ ID NO: 483 - protein, 36 aa, synthetic construct
GGGSGGSVPLSLYSGGGISSGLLSGRSDNHGGSGGS

SEQ ID NO: 484 - protein, 36 aa, synthetic construct
GGGSHPVGLLARGGGHPVGLLARGGGHPVGLLARGS

SEQ ID NO: 485 - protein, 36 aa, synthetic construct
GGGSHPVGLLARGGGHPVGLLARGGSGRSAGGSGRS